SEQUENCE LISTING (38 sequences)

SEQ ID NO: 1 - protein, 7 aa, synthetic construct
GYTFTGY

SEQ ID NO: 2 - protein, 6 aa, synthetic construct
NPYNDD

SEQ ID NO: 3 - protein, 15 aa, synthetic construct
GAGYNFDGAYRFFDF

SEQ ID NO: 4 - protein, 16 aa, synthetic construct
RSSQRLVHSNGNTYLH

SEQ ID NO: 5 - protein, 7 aa, synthetic construct
RVSNRFP

SEQ ID NO: 6 - protein, 9 aa, synthetic construct
SQSTHVPYT

SEQ ID NO: 7 - protein, 124 aa, Mus musculus
EVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSS

SEQ ID NO: 8 - protein, 113 aa, Mus musculus
DVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKR

SEQ ID NO: 9 - protein, 124 aa, synthetic construct
QVQLVQSGAEVKKPGASVKVSCKASGYTFTGYVMHWVRQAPGQGLEWMGFINPYNDDIQSNERFRGRVTMTRDTSISTAYMELSRLRSDDTAVYYCARGAGYNFDGAYRFFDFWGQGTMVTVSS

SEQ ID NO: 10 - protein, 124 aa, synthetic construct
EVQLVESGAEVKKPGASVKVSCKASGYTFTGYVMHWVRQAPGQGLEWMGFINPYNDDIQSNERFRGRVTMTRDTSTSTVYMELSSLRSEDTAVYYCARGAGYNFDGAYRFFDFWGQGTLVTVSS

SEQ ID NO: 11 - protein, 124 aa, synthetic construct
QVQLVQSGAEVKKPGASVKVSCKASEYSFTGYVMHWVRQAPGQGLEWMGFINPYNDDIQSNERFRGRVTMTRDTSISTAYMEVSSLTSDDAAIYYCARGAGYNFDGAYRFFDFWGQGTLVTVSS

SEQ ID NO: 12 - protein, 124 aa, synthetic construct
QVQLVQSGAEVKKPGASVKVSCKASGYTFTGYVMHWVRQAPGQGLEWMGFINPYNDDIQSNERFRGRVTMTRDKSISTAYMELSRLRSDDTAVYYCARGAGYNFDGAYRFFDFWGQGTMVTVSS

SEQ ID NO: 13 - protein, 124 aa, synthetic construct
QVQLVQSGAEVKKPGASVKVSCKASGYTFTGYVMHWVRQAPGQGLEWMGFINPYNDDIQSNERFRGRVTMTSDTSISTAYMELSRLRSDDTAVYYCARGAGYNFDGAYRFFDFWGQGTMVTVSS

SEQ ID NO: 14 - protein, 124 aa, synthetic construct
QVQLVQSGAEVKKPGASVKVSCKASGYTFTGYVMHWVRQAPGQGLEWMGFINPYNDDIQSNERFRGRVTMTSDKSISTAYMELSRLRSDDTAVYYCARGAGYNFDGAYRFFDFWGQGTMVTVSS

SEQ ID NO: 15 - protein, 124 aa, synthetic construct
QVQLVQSGAEVKKPGASVKVSCKASGYTFTGYVMHWVRQAPGQGLEWIGFINPYNDDIQSNERFRGRVTMTRDTSISTAYMELSRLRSDDTAVYYCARGAGYNFDGAYRFFDFWGQGTMVTVSS

SEQ ID NO: 16 - protein, 124 aa, synthetic construct
QVQLVQSGAEVKKPGASVKVSCKASGYTFTGYVMHWVRQAPGQGLEWIGFINPYNDDIQSNERFRGRVTMTRDKSISTAYMELSRLRSDDTAVYYCARGAGYNFDGAYRFFDFWGQGTMVTVSS

SEQ ID NO: 17 - protein, 124 aa, synthetic construct
QVQLVQSGAEVKKPGASVKMSCKASGYTFTGYVMHWVRQAPGQGLEWMGFINPYNDDIQSNERFRGRVTMTSDKSISTAYMELSRLRSDDTAVYYCARGAGYNFDGAYRFFDFWGQGTMVTVSS

SEQ ID NO: 18 - protein, 124 aa, synthetic construct
QVQLVQSGAEVKKPGASVKMSCKASGYTFTGYVMHWVRQAPGQGLEWIGFINPYNDDIQSNERFRGRVTMTRDTSISTAYMELSRLRSDDTAVYYCARGAGYNFDGAYRFFDFWGQGTMVTVSS

SEQ ID NO: 19 - protein, 112 aa, synthetic construct
DIVMTQSPLSLPVTPGEPASISCRSSQRLVHSNGNTYLHWYLQKPGQSPRLLIYRVSNRFPGVPDRFSGSGSGTDFTLKISRVEAEDVGVYYCSQSTHVPYTFGQGTKLEIK

SEQ ID NO: 20 - protein, 112 aa, synthetic construct
DIVMTQSPLSLPVTLGEQASISCRSSQRLVHSNGNTYLHWYLQKPGQSPRLLIYRVSNRFPGVPDRFSGSGSGTDFTLKISRVEAEDVGVYFCSQSTHVPYTFGGGTKLEIK

SEQ ID NO: 21 - protein, 112 aa, synthetic construct
DIVMTQSPLSLPVTLGEQASISCRSSQRLVHSNGNTYLHWYLQKPGQSPRLLIYRVSNRFPGVPDRFSGSGSGTDFTLKISRVEAEDVGVYFCSQSTHVPYTFGQGTKLEIK

SEQ ID NO: 22 - protein, 112 aa, synthetic construct
DIVMTQSPLSLPVTLGEQASISCRSSQRLVHSNGNTYLHWYLQKPGQSPRLLIYRVSNRFPGVPDRFSGSGSGTDFTLKISRVEAEDVGVYYCSQSTHVPYTFGGGTKLEIK

SEQ ID NO: 23 - protein, 112 aa, synthetic construct
DIVMTQSPLSLPVTLGEQASISCRSSQRLVHSNGNTYLHWYLQKPGQSPRLLIYRVSNRFPGVPDRFSGSGSGTDFTLKISRVEAEDVGVYYCSQSTHVPYTFGQGTKLEIK

SEQ ID NO: 24 - protein, 112 aa, synthetic construct
DIVMTQSPLSLPVTLGEPASISCRSSQRLVHSNGNTYLHWYLQKPGQSPRLLIYRVSNRFPGVPDRFSGSGSGTDFTLKISRVEAEDVGVYFCSQSTHVPYTFGGGTKLEIK

SEQ ID NO: 25 - protein, 112 aa, synthetic construct
DIVMTQSPLSLPVTLGEPASISCRSSQRLVHSNGNTYLHWYLQKPGQSPRLLIYRVSNRFPGVPDRFSGSGSGTDFTLKISRVEAEDVGVFYCSQSTHVPYTFGQGTKLEIK

SEQ ID NO: 26 - protein, 112 aa, synthetic construct
DIVMTQSPLSLPVTLGEPASISCRSSQRLVHSNGNTYLHWYLQKPGQSPRLLIYRVSNRFPGVPDRFSGSGSGTDFTLKISRVEAEDVGVYYCSQSTHVPYTFGGGTKLEIK

SEQ ID NO: 27 - protein, 112 aa, synthetic construct
DIVMTQSPLSLPVTLGEPASISCRSSQRLVHSNGNTYLHWYLQKPGQSPRLLIYRVSNRFPGVPDRFSGSGSGTDFTLKISRVEAEDVGVYYCSQSTHVPYTFGQGTKLEIK

SEQ ID NO: 28 - protein, 112 aa, synthetic construct
DIVMTQSPLSLPVTPGEQASISCRSSQRLVHSNGNTYLHWYLQKPGQSPRLLIYRVSNRFPGVPDRFSGSGSGTDFTLKISRVEAEDVGVYFCSQSTHVPYTFGGGTKLEIK

SEQ ID NO: 29 - protein, 112 aa, synthetic construct
DIVMTQSPLSLPVTPGEQASISCRSSQRLVHSNGNTYLHWYLQKPGQSPRLLIYRVSNRFPGVPDRFSGSGSGTDFTLKISRVEAEDVGVYFCSQSTHVPYTFGQGTKLEIK

SEQ ID NO: 30 - protein, 112 aa, synthetic construct
DIVMTQSPLSLPVTPGEQASISCRSSQRLVHSNGNTYLHWYLQKPGQSPRLLIYRVSNRFPGVPDRFSGSGSGTDFTLKISRVEAEDVGVYYCSQSTHVPYTFGGGTKLEIK

SEQ ID NO: 31 - protein, 112 aa, synthetic construct
DIVMTQSPLSLPVTPGEQASISCRSSQRLVHSNGNTYLHWYLQKPGQSPRLLIYRVSNRFPGVPDRFSGSGSGTDFTLKISRVEAEDVGVYYCSQSTHVPYTFGQGTKLEIK

SEQ ID NO: 32 - protein, 112 aa, synthetic construct
DIVMTQSPLSLPVTPGEPASISCRSSQRLVHSNGNTYLHWYLQKPGQSPRLLIYRVSNRFPGVPDRFSGSGSGTDFTLKISRVEAEDVGVYFCSQSTHVPYTFGGGTKLEIK

SEQ ID NO: 33 - protein, 112 aa, synthetic construct
DIVMTQSPLSLPVTPGEPASISCRSSQRLVHSNGNTYLHWYLQKPGQSPRLLIYRVSNRFPGVPDRFSGSGSGTDFTLKISRVEAEDVGVYFCSQSTHVPYTFGQGTKLEIK

SEQ ID NO: 34 - protein, 112 aa, synthetic construct
DIVMTQSPLSLPVTPGEPASISCRSSQRLVHSNGNTYLHWYLQKPGQSPRLLIYRVSNRFPGVPDRFSGSGSGTDFTLKISRVEAEDVGVYYCSQSTHVPYTFGGGTKLEIK

SEQ ID NO: 35 - protein, 650 aa, synthetic construct
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLVQSGAEVKKPGASVKVSCKASGYTFTGYVMHWVRQAPGQGLEWMGFINPYNDDIQSNERFRGRVTMTRDTSISTAYMELSRLRSDDTAVYYCARGAGYNFDGAYRFFDFWGQGTMVTVSSGGGGSGGGGSGGGGSDIVMTQSPLSLPVTPGEPASISCRSSQRLVHSNGNTYLHWYLQKPGQSPRLLIYRVSNRFPGVPDRFSGSGSGTDFTLKISRVEAEDVGVYYCSQSTHVPYTFGQGTKLEIKRSDPAEPKSPDKTHTCPPCPKDPKIIAIAVVGALLLVALIFGTASYLIKRGRKKLLYIFKQPFMRPVQTTQEEDGCSCRFPEEEEGGCELRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 5 aa, synthetic construct
GGGGS

SEQ ID NO: 37 - protein, 175 aa, Homo sapiens
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKD

SEQ ID NO: 38 - protein, 175 aa, Homo sapiens
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKD

(19) United States
(12) Patent Application Publication
(10) Pub. No.: US 2026/0263599 A1
(43) Pub. Date: Sep. 10, 2026

(54) TREATMENT OF TRBC1-POSITIVE T CELL MALIGNANCIES

(71) Applicant: AUTOLUS LIMITED, London (GB)

(72) Inventors: Martin Pulé, London (GB); Vijay Peddareddigari, London (GB)

(21) Appl. No.: 19/491,316

(22) PCT Filed: Jun. 13, 2024
(86) PCT No.: PCT/GB2024/051514

§ 371 Date: Dec. 8, 2025

(30) Foreign Application Priority Data
Jun. 14, 2023 (GB) 2308906.3
Jul. 31, 2023 (GB) 2311746.8

(51) Int. Cl. A61K 40/42; A61K 40/11; A61K 40/31; A61P 35/00

(52) U.S. Cl. CPC A61K 40/421; A61K 40/11; A61K 40/31; A61P 35/00

(57) ABSTRACT

The present disclosure relates to the treatment of T Cell Receptor Beta Constant 1 (TRBC1)-positive T-cell malignancies with CAR T cells targeting TRBC1.

### FIELD

The present disclosure relates to the treatment of T Cell Receptor Beta Constant 1 (TRBC1)-positive T-cell malignancies with CAR T cells targeting TRBC1.

### BACKGROUND

Lymphoid malignancies can largely be divided into those which are derived from either T-cells or B-cells. T-cell malignancies are a clinically and biologically heterogeneous group of disorders, together comprising 10-20% of non-Hodgkin's lymphomas and 20% of acute leukaemias. The most commonly identified histological subtypes are peripheral T-cell lymphoma, not otherwise specified (PTCL-NOS); angio-immunoblastic T-cell lymphoma (AITL) and anaplastic large cell lymphoma (ALCL). Of all acute Lymphoblastic Leukaemias (ALL), some 20% are of a T-cell phenotype.

These conditions typically behave aggressively, compared for instance with B-cell malignancies, with estimated 5-year survival of only 30%. In the case of T-cell lymphoma, they are associated with a high proportion of patients presenting with disseminated disease, unfavourable International Prognostic Indicator (IPI) score and prevalence of extra-nodal disease. Chemotherapy alone is not usually effective and less than 30% of patients are cured with current treatments.

In the frontline setting, most patients with PTCL are treated with multi-agent anthracycline-based chemotherapy regimens such as cyclophosphamide-doxorubicin-oncovin (vincristine)-prednisone (CHOP). A systematic meta-analysis evaluated CHOP or CHOP-like regimens in 2815 patients with PTCL. The complete response (CR) rates associated with anthracycline-based regimens ranged from 30% to 76% across studies and subtypes of PTCL. As expected, ALCL showed a higher CR rate with anthracycline-based chemotherapy than other T cell lymphomas; in patients with Angioimmunoblastic T cell lymphoma, a CR rate of 36% to 70% was seen and in patients with PTCL-NOS, 44% to 64% was observed. The 5-year overall survival (OS) was of 38.5% (Abouyabis et al. 2011, ISRN Hematol 2011: 623924). Autologous hematopoietic cell transplantation is incorporated into the initial treatment of patients with PTCL as consolidation therapy after initial combination chemotherapy.

Most patients with PTCL will however either not achieve remission or will relapse after first line therapy (Dreyling et al. 2013, Ann Oncol 24(4):857-877). Given the paucity of data and limited therapeutic options, participation in clinical trials is recommended (National Comprehensive Cancer Network PTCL Guidelines 2016, ESMO consensus guidelines). Most patients with relapsed or refractory PTCL have poor outcomes with short survival. In a study of 153 patients (Mak et al. 2013, J Clin Oncol 31(16):1970-1976) with PTCL-NOS, Angioimmunoblastic T cell lymphoma and Anaplastic large cell lymphoma (both ALK+ and ALK−) and in the absence of hematopoietic stem-cell transplantation, the median time from initial diagnosis to relapse or progression after first treatment was only 6.7 months. The median overall survival and median progression-free survival after relapse or progression were 5.5 and 3.1 months respectively. This was minimally improved in patients who received chemotherapy at relapse with median overall survival and median progression-free survival at 6.5 and 3.7 months respectively. For subsets such as ALCL, with newer treatment options such as brentuximab, outcomes have shown incremental improvement (Pro et al. 2012, J Clin Oncol 30(18):2190-2196), but overall the prognosis for most PTCL subtypes in the relapse or refractory setting remains poor.

Conventional platinum-based regimens such as DHAP, ESHAP or ICE, as used in the larger Diffuse Large B Cell lymphoma population, are used in patients with relapsed PTCL especially those who are transplant candidates. The efficacy of these regimens in PTCL is not well known as no large, published study is available in these lymphomas. The Canadian Cancer Trials Group LY.12 Phase 3 trial included 59 patients with PTCL. Among these, 81% had advanced stage disease, and 41% were refractory to primary therapy. The ORR after two cycles of salvage chemotherapy was 36%; no difference was observed between dexamethasone, cytarabine, cisplatin (33%), and gemcitabine, cisplatin, dexamethasone (38%) therapy. At one year, event-free survival (EFS) was 16% and OS was 28% (Skamene et al. 2017, Leuk Lymphoma 58(10):2319-2327). Patients who received autologous stem cell transplant (SCT), two-year EFS and OS were 21% and 42%, respectively.

Patients with PTCL had inferior OS and outcomes when compared to patients with B cell lymphomas.

Newer agents are being evaluated and have shown some marginal efficacy in patients with relapsed PTCL. A single arm Phase 2 PROPEL study of pralatrexate evaluated 115 heavily pre-treated patients with the most common types of PTCL. In this study, the ORR was 29% with CR/CRu (complete response unconfirmed) of 11%; the median PFS and OS was 3.5 and 14.5 months respectively. Responses were observed across all histologic subtypes, although patients with Angioimmunoblastic T cell lymphoma were less likely to respond than patients with other common PTCL subtypes (O'Connor et al. 2011, J Clin Oncol 29(9):1182-1189).

Histone deacetylase (HDAC) inhibitors such as belinostat and romidepsin have shown modest efficacy in patients with PTCL. In the single arm Phase 2 BELIEF study with belinostat, a total of 129 patients were enrolled, with a median of two prior systemic therapies. ORR and CR in the 120 evaluable patients was 26%, 11% respectively. Median duration of response was 13.6 months, whilst median PFS and OS were 1.6 and 7.9 months respectively (O'Connor et al. 2015, J. Clin. Oncol. 33(23):2492-2499).

Similarly, in the single arm Phase 2 study of romidepsin, 130 patients with histologically confirmed PTCL who had relapsed or were refractory to least one prior therapy were treated. The ORR was 25% including 15% CR/CRu. The median duration of response was 17 months (Coiffier et al. 2012, J Clin Oncol 30(6):631-636).

For patients with CD30 positive ALCL, newer treatment options such as anti-CD30 antibody drug conjugate brentuximab have shown incremental improvement in outcomes (Pro et al. 2012, J Clin Oncol 30(18):2190-2196). In this Phase 2 study of 58 patients the ORR was 86% and 57% achieved a CR. The median durations of overall response and CR were 12.6 and 13.2 months, respectively.

With exception of brentuximab, these newer agents are only approved by the Food and Drug Administration (FDA) and are not readily available elsewhere.

Relapses in patients with PTCL are common; therefore, SCT as a consolidation and salvage strategy may improve therapeutic results. However, the role of SCT especially in relapsed PTCL remains to be determined and there is lack of prospective clinical trials. Some limited retrospective studies have evaluated the efficacy of autologous and allogeneic transplant in relapsed PTCL. For example, a longitudinal study of 241 patients with PTCL NOS, ALCL and angioimmunoblastic T cell lymphoma has shown that SCT is better in the earlier disease setting with chemo-sensitive disease when used as consolidation therapy at first CR (Smith 2013, J. Med. Toxicol. 9(4):355-369).

An important difficulty in the development of immunotherapy for T-cell disorders is the considerable overlap in marker expression of clonal and normal T-cells. Unlike in B-cell malignancies, where depletion of the B-cell compartment results in relatively minor immunosuppression which is readily tolerated by most patients or, in therapies which result in particularly long-term depletion of the normal B-compartment, its loss can be largely abrogated by administration of pooled immunoglobulin, the situation is completely different when targeting T-cell malignancies. Depletion of the T-cell compartment leads to severe immunosuppression and severe toxicity. Further, there is no satisfactory way to mitigate loss of the T-cell compartment. The toxicity is in part illustrated by the clinical effects of the therapeutic monoclonal antibody Alemtuzumab. This agent lyses cells which express CD52 and has some efficacy in T-cell malignancies. The utility of this agent is greatly limited by a profound cellular immunodeficiency, largely due to T-cell depletion, with markedly elevated risk of infection.

Few studies to date have reported on use of CAR-T cell therapy (CAR-T) in PTCL. Anti-CD7 CAR-T have been tested in T acute lymphoblastic leukaemia (T-ALL), with high response rates. CD7 is a pan-T cell antigen also expressed on natural killer (NK) cells, but expression is frequently lost in PTCL with only approximately 25% of tumours expressing CD7 [Went et al. J. Clin. Oncol. Off. J. Am. Soc. Clin. Oncol. 24, 2472-2479 (2006)]. Early data with CAR-T targeting CD5, another pan-T antigen which is expressed on 20-90% of cases of PTCL [Went et al., 2006; Patel et al. Iran. J. Blood Cancer 11, (2019)] has been reported in PTCL. Two of 10 patients achieved a transient CR, with short CAR-T persistence noted [Hill et al. Safety and Anti-Tumor Activity of CD5 CAR T-Cells in Patients with Relapsed/Refractory T-Cell Malignancies. Blood 134, 199 (2019)]; this study continues with a revised manufacturing process. A CAR-T cell study targeting CD4 was stopped. These pan-T cell targets are limited by expression on healthy T cells resulting in CAR-T cell fratricide and immunosuppression caused by depletion of normal T cells. In addition, T cell lymphomas frequently have aberrant downregulation of one or more of these markers (Went et al., 2006). Other targets such as CD30, CD37, or CD70, which have none or limited expression on normal T cells can be used: however, these are only expressed on a small proportion of PTCL cases.

There is thus a need for a new method for targeted treatment of T-cell malignancies.

### BRIEF DESCRIPTION OF THE DRAWINGS

This patent or application file contains at least one drawing executed in color. Copies of this patent or patent application publication with color drawing(s) will be provided by the United States Patent and Trademark Office upon request and payment of the necessary fee.

FIG. 1: A diagram of the αβ T-cell Receptor/CD3 Complex. The T-cell receptor is formed from 6 different protein chains which must assemble in the endoplasmic reticulum to be expressed on the cell surface. The four proteins of the CD3 complex (CD3ζ, CD3γ, CD3ε and CD3δ) sheath the T-cell Receptor (TCR). This TCR imbues the complex with specificity of a particular antigen and is composed of two chains: TCRα and TCRβ. Each TCR chain has a variable component distal to the membrane and a constant component proximal to the membrane. Nearly all T-cell lymphomas and many T-cell leukaemias express the TCR/CD3 complex.

FIG. 2: The segregation of T-cell Receptor β-constant region (TRBC)-1 and TRBC2 during T-cell receptor rearrangement. Each TCR beta chain is formed from genomic recombination of a particular beta variable (V), diversity (D), joining (J) and constant (TRBC) regions. The human genome contains two very similar and functionally equivalent TRBC loci known αβ TRBC1 and TRBC2. During TCR gene re-arrangement, a J-region recombines with either TRBC1 or TRBC2. This rearrangement is permanent. T-cells express many copies of a single TCR on their surface, hence each T-cell will express a TCR whose β-chain constant region is coded for by either TRBC1 or TRBC2.

FIG. 3: Alignment of human TRBC1 and TRBC2 at the amino acid level. The TCRβ constant chain coded for by TRBC1 and TRBC2 differ by only 4 amino acid differences: K/N at position 3 of the TRBC; N/K at position 4 of the TRBC; F/Y at position 36 of the TRBC; V/E at position 135 of the TRBC.

FIG. 4: Schematic diagrams illustrating different antibody types referred to in the generation of humanised anti-TRBC1 binders.

FIG. 5: Heavy and light chain graft selection. Humanised VH and VL domains were created comprising the CDRs from JOVI-1 together with various human framework regions. Chimeric antibodies were generated comprising humanised VH with murine VL domains, or humanised VL with murine VH domains and compared with a control chimeric antibody having murine VH and VL (Jovi-1 chimera HC/Jovi-1 chimera LC). Humanised antibodies were also created with humanised VH and VL combinations. All antibodies were tested for binding to TRBC1 by ELISA.

FIG. 6: TRBC1/TRBC2 binding of back-mutated constructs. A series of back-mutated VH constructs were created based on the H-AF062256 framework as shown in Table 1. These VH domains were used to create humanised antibodies in combination with a VL domain having the 3aaz human framework. The antibodies were tested for binding to TRBC1 and TRBC2 by ELISA.

FIG. 7: Schematic diagram illustrating a humanised anti-TRBC1 chimeric antigen receptor (CAR)

FIG. 8: Flow diagrams showing two processes for manufacturing autologous AUTO4 (anti-TRBC1 CAR T-cell) products. Process B involving fresh apheresis, optimization of seeding and transduction, and CD4+/CD8+ selection is designed to reduce manufacturing time and produce CAR T-cells with a more naïve and central memory phenotype with greater proliferative potential.

FIG. 9: Schematic diagram showing the number of treated patients and the dose received by patients of anti-TRBC1 CAR T-cell products produced by Process A or Process B shown in FIG. 8.

FIG. 10: Blood absolute lymphocyte counts in treated patients.

FIG. 11: Detection via immunohistochemistry of CAR T-cells in lymph node biopsies from treated patients humanised.

FIG. 12: Greatest change in SPD in response-evaluable treated patients.

FIG. 13: Efficacy of patient treatment with Process A AUTO4.

FIG. 14: PET-CT in responding treated patients.

FIG. 15: Efficacy of patient treatment with Process B AUTO4.

FIG. 16: Annotated amino acid sequence of the anti-TRBC1 CAR of AUTO4.

FIG. 17: Consort diagram.

FIG. 18: AUTO4 drug product characteristics. a) Diagram of manufacturing process for AUTO4 CAR-T cells. b) Flow cytometric analysis of cryopreserved CAR-T cell drug products for patients treated using a panel to determine product exhaustion. Markers included were PD1, TM3, LAG3 and TIGIT. “0” denotes cells not expressing any of the markers; “1” includes cells expressing a single marker; “2” includes cells expressing any two markers in combination; “3” includes any three in combination or all four markers. c) Flow cytometric analysis of cryopreserved CAR-T cell drug products for patients treated. Immunophenotyping panel included CD45RA and CCR7. CCR7⁺ CD45RA⁺ were considered naïve cells; CCR7⁺ CD45RA⁻ T cells were considered central memory; CCR7⁻ CD45RA⁻ were considered Effector memory cells and CCR7⁻ CD45RA⁺ cells were considered Terminally differentiated effector memory T cell.

FIG. 19: Peripheral blood counts for first 3 months. (a) Lymphocytes; (b) Neutrophils and (c) Platelets

FIG. 20: Peripheral blood TRBC1 percentage. a) TRBC1 and TRBC2% in peripheral blood determined by flow cytometry. b) CD4:CD8 ratio in peripheral blood determined by flow cytometry.

FIG. 21: Serum cytokines: Cytokine levels measured in peripheral blood for TNFα, GM-CSF, IFNγ, IL-2, IL-5, IL-6, IL-7, IL-8, IL-10 and IL-15.

FIG. 22: Clinical trial cohort. (a) Swim plot showing outcome in patients who received AUTO4. Note, one patient (14) who received 225×10⁶ AUTO4 CAR T cells, was in CMR following bridging at time of infusion and is marked NE for non-evaluable; (b) 18FDG PET CT imaging prior to AUTO4, at month 1 and at 12 months from two subjects [47 and 55] in long term CMR following AUTO4.

FIG. 23: Kaplan-Meier plot of PFS. Progression-free survival (PFS) based on Overall Response (Lugano Classification). Median with 95% CI calculated from PROC LIFETEST output. Time relative to first AUTO4 treatment. 1 month=30.4375 days.

FIG. 24: Lymph node biopsy

Lymph node biopsy post AUTO4 infusion (patient 01 day 19). a) Formalin fixed, paraffin-embedded tissue sections of a T cell lymphoma stained by double immunofluorescence with anti-CD34 (Q/BenD10), which stains RQR8 (red), and CD3 (yellow) that detects T cells. DAPI (blue) is used to nuclear counterstaining. CAR T cells (orange) are identified by co-expression of both Q/BendD10 and anti-CD3. b) Magnification of orange circled cell in (a) showing overlay (top), CD3 staining (centre), and CD34 staining (bottom).

FIG. 25: Lymph node biopsies. Lymph node biopsies post AUTO4 infusion for patient ID 09 (day 100), 22 (day 7), 55 (day 12), and 59 (day 8). (main image) Formalin fixed, paraffin-embedded tissue sections of a T cell lymphoma stained by double immunofluorescence with anti-CD34 (Q/BenD10), which stains RQR8 (red), and CD3 (yellow) that detects T cells. DAPI (blue) is used to nuclear counterstaining. CAR T cells (orange) are identified by co-expression of both Q/BendD10 and anti-CD3. (Right hand panels) magnifications of orange circled cells showing overlay (top), CD3 staining (centre), and CD34 staining (bottom).

FIG. 26: In vitro cytotoxicity. a) In vitro cytotoxicity assay on Jurkat TRBC1 and TCR KO cell lines, and TRBC1⁺ and TRBC2⁺ healthy human T cells, with HuJovi1-hinge-TyrpTM-41BBz CAR (AUTO4) and control anti-CD19 CAR (CD8stk-CD8TM-41BBz). E:T ratios 1:1, 1:2, 1:4 and 1:8, 72 h, n=6. Multiple paired t test. **=P<0.01, ***=P<0.001. b) Reverse killing assay for healthy human T cell (TRBC1⁺ or TRBC2⁺) with HuJovi1-hinge-TyrpTM-41BBz CAR (AUTO4) and control anti-CD19 CAR (CD8stk-CD8TM-41 BBz). E:T ratios 4:1, 1:1 and 1:4, 72 h, n=6. Multiple paired t test. *=P<0.05, **=P<0.01.

### SUMMARY

The disclosure provides methods for treating a TRBC1-positive malignancy in a patient comprising administering to the patient autologous anti-TRBC1 CAR T-cells (for example, the “AUTO4” autologous anti-TRBC1 CAR T-cell product comprising anti-TRBC1 CARs described in Example 8 herein).

Methods are provided wherein the age of the patient is eighteen years or older.

Methods are provided wherein the T-cell malignancy includes but is not limited to: peripheral T-cell lymphoma, not otherwise specified (PTCL-NOS); angio-immunoblastic T-cell lymphoma (AITL); or anaplastic large cell lymphoma (ALCL). Other T-cell malignancies include but are not limited to: enteropathy-associated T-cell lymphoma (EATL), hepatosplenic T-cell lymphoma (HSTL), extranodal NK/T-cell lymphoma nasal type, cutaneous T-cell lymphoma, primary cutaneous ALCL, T cell prolymphocytic leukaemia and T-cell acute lymphoblastic leukaemia.

Methods are provided in particular wherein the patient has: a) relapsed or refractory T-cell malignancy following at least one line of therapy, and b) confirmed TRBC1-positive tumor.

In the methods provided, the patient may be administered a single dose of about 25×10⁶, 75×10⁶, 225×10⁶, 450×10⁶, or 900×10⁶ anti-TRBC1 CAR T-cells. The patient may be administered a single dose of about 450×10⁶ anti-TRBC1 CAR T-cells. The administration may be an intravenous injection through a Hickman line or peripherally inserted central catheter.

Methods are provided wherein the anti-TRBC1 CAR T-cells express a chimeric antigen receptor (CAR) comprising a TRBC1-binding domain which comprises

a) a heavy chain variable region (VH) having
CDRs (Chothia definition) comprising the
following sequences:
VH CDR1:
(SEQ ID No. 1)
GYTFTGY,
VH CDR2:
(SEQ ID No. 2)
NPYNDD
and
VH CDR3:
(SEQ ID No. 3)
GAGYNFDGAYRFFDF;
and
b) a light chain variable region (VL) having
CDRs (Chothia definition) comprising the
following sequences:
VL CDR1:
(SEQ ID No. 4)
RSSQRLVHSNGNTYLH,
VL CDR2:
(SEQ ID No. 5)
RVSNRFP
and
VL CDR3:
(SEQ ID No. 6)
SQSTHVPYT.

The CDRs may be grafted on to a human antibody framework. In provided methods, the anti-TRBC1 binding domain may comprise a VH domain having the sequence shown as SEQ ID NO: 9 and/or or a VL domain having the sequence shown as SEQ ID NO: 19 or a variant thereof having at least 95% sequence identity.

The anti-TRBC1 binding domain may comprise an scFv in the orientation VH-VL or VL-VH.

The anti-TRBC1 binding domain may comprise a Fab. The anti-TRBC1 binding Fab may comprise a heavy chain (VH-CH1) sequence shown as SEQ ID NO: 9. The anti-TRBC1 binding Fab may comprise a light chain (VL-CL kappa) sequence shown as SEQ ID NO: 19. The CDR sequences (Kabat definition) are underlined in the sequences below.

SEQ ID No. 9
QVQLVQSGAEVKKPGASVKVSCKASGYTFTGYVMHVVVRQAPGQG
LEVVMGFINPYNDDIQSNERFRGRVTMTRDTSISTAYMELSRLRS
DDTAVYYCARGAGYNFDGAYRFFDFWGQGTMVTVSS
SEQ ID No. 19
DIVMTQSPLSLPVTPGEPASISCRSSQRLVHSNGNTYLHVVYLQK
PGQSPRLLIYRVSNRFPGVPDR FSGSGSGTDFTLKISRVEAEDV
GVYYCSQSTHVPYTFGQGTKLEI K

The anti-TRBC1 binding domain and a transmembrane domain may be connected in the CAR by a spacer such as a human IgG1 hinge. The CAR may comprise intracellular T cell signaling domain such as an intracellular T-cell signaling domain comprising the 41BB endodomain and the CD3-Zeta endodomain.

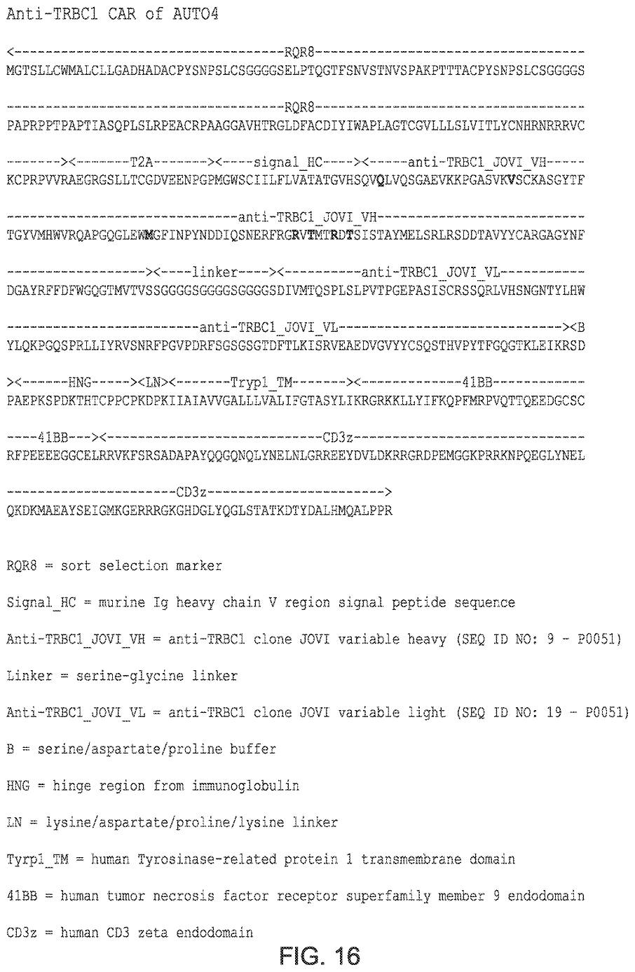

An anti-TRBC1 CAR provided herein may have the amino acid sequence shown in FIG. 16 and also in SEQ ID No. 35.

It is contemplated herein that the antigen-binding domains can be used in a variety of therapeutic formats, including but not limited to a chimeric antigen receptor (CAR), therapeutic antibody, antibody-drug conjugate (ADC) and bi-specific T cell engager (BiTE) to deplete malignant TRBC1-expressing T-cells in a subject, without affecting healthy TRBC2-expressing T cells.

Thus in a first aspect, the disclosure provides anti-TRBC1 antigen-binding domain which comprises:
a) a VH domain having an amino acid sequence selected from SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17 and SEQ ID No. 18; and
b) a VL domain having an amino acid sequence selected from SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 29, SEQ ID No. 30, SEQ ID No. 31, SEQ ID No. 32, SEQ ID No. 33, SEQ ID No. 34.

In a second aspect, the disclosure provides a chimeric antigen receptor (CAR) which comprises an anti-TRBC1 antigen binding domain according to the first aspect of the disclosure.

In a third aspect, the disclosure provides an antibody which comprises an anti-TRBC1 antigen binding domain according to the first aspect of the disclosure.

In a fourth aspect, the disclosure provides a bispecific T-cell engager (BiTE) which comprises an anti-TRBC1 antigen binding domain according to the first aspect of the disclosure.

In a fifth aspect, the disclosure provides antibody-drug conjugate which comprises an anti-TRBC1 antigen binding domain according to the first aspect of the disclosure.

In a sixth aspect, the disclosure provides a nucleic acid sequence which encodes a CAR according to the second aspect of the disclosure.

In a seventh aspect, the disclosure provides a vector comprising a nucleic acid sequence according to the sixth aspect the disclosure.

In an eighth aspect, the disclosure provides cell comprising a CAR according to the second aspect of the disclosure.

In a ninth aspect, the disclosure provides a method for making a cell according to the eighth aspect of the disclosure, which comprises the step of introducing a nucleic acid according to the sixth aspect of the disclosure or a vector according to the seventh aspect of the disclosure into a cell.

In a tenth aspect, the disclosure provides a pharmaceutical composition which comprises a plurality of cells according to the eighth aspect of the disclosure, an antibody according to the third aspect of the disclosure, a BiTE according to the fourth aspect of the disclosure or an antibody-drug conjugate according to the fifth aspect of the disclosure.

In an eleventh aspect, the disclosure provides a pharmaceutical composition according to the tenth aspect of the disclosure for use in treating a TRBC1-expressing T-cell lymphoma or leukaemia in a subject.

In a twelfth aspect, the disclosure provides a method for treating a TRBC1-expressing T-cell lymphoma or leukaemia in a subject, which comprises the step of administering a pharmaceutical composition according to the tenth aspect of the disclosure to a subject.

In a thirteenth aspect, the disclosure provides the use of a pharmaceutical composition according to the tenth aspect of the disclosure in the manufacture of a medicament for treating a TRBC1-expressing T-cell lymphoma or leukaemia in a subject.

The TRBC1-expressing T-cell lymphoma or leukaemia may be, for example: peripheral T-cell lymphoma, not otherwise specified (PTCL-NOS); angio-immunoblastic T-cell lymphoma (AITL), anaplastic large cell lymphoma (ALCL), enteropathy-associated T-cell lymphoma (EATL), hepatosplenic T-cell lymphoma (HSTL), extranodal NK/T-cell lymphoma nasal type, cutaneous T-cell lymphoma, primary cutaneous ALCL, T cell prolymphocytic leukaemia or T-cell acute lymphoblastic leukaemia.

### DETAILED DESCRIPTION

The disclosure provides agents, such as chimeric antigen receptors (CARs) which selectively bind TRBC1. As described in WO2015/132598 and WO2018/224844, such agents are useful in methods for treating a T-cell lymphoma or leukaemia in a subject. T cell malignancies are clonal, so they either express TRBC1 or TRBC2. By administering a TRBC1 selective agent to the subject, the agent causes selective depletion of the TRBC1-expressing malignant T-cells, together with TRBC1-expressing normal T-cells, but does not cause depletion of TRBC2-expressing normal T-cells.

### TCR β Constant Region (TRBC)

The T-cell receptor (TCR) is expressed on the surface of T lymphocytes and is responsible for recognising antigens bound to major histocompatibility complex (MHC) molecules. When the TCR engages with antigenic peptide and MHC (peptide/MHC), the T lymphocyte is activated through a series of biochemical events mediated by associated enzymes, co-receptors, specialized adaptor molecules, and activated or released transcription factors.

The TCR is a disulfide-linked membrane-anchored heterodimer normally consisting of the highly variable alpha (α) and beta (β) chains expressed as part of a complex with the invariant CD3 chain molecules. T-cells expressing this receptor are referred to as α:β (or αβ) T-cells (~95% total T-cells). A minority of T-cells express an alternate receptor, formed by variable gamma (γ) and delta (δ) chains, and are referred to as γδ T-cells (~5% total T cells).

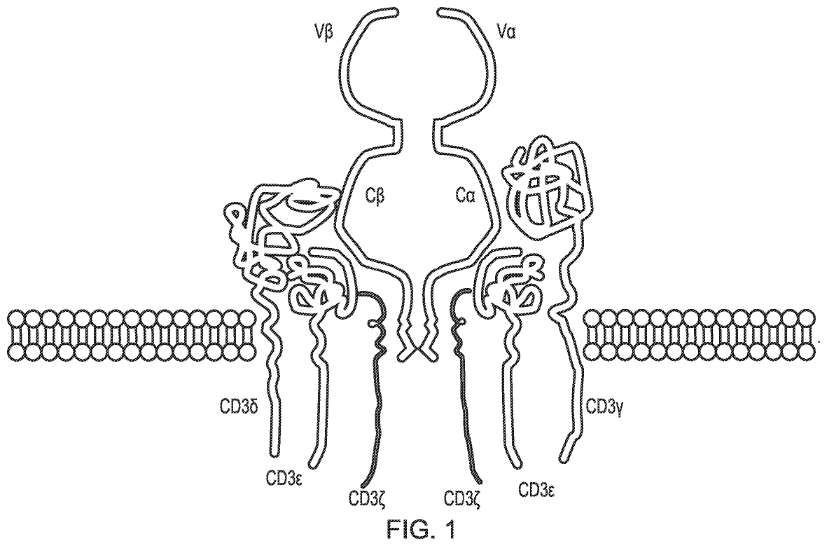

Each α and β chain is composed of two extracellular domains: Variable (V) region and a Constant (C) region, both of Immunoglobulin superfamily (IgSF) domain forming antiparallel β-sheets. The constant region is proximal to the cell membrane, followed by a transmembrane region and a short cytoplasmic tail, while the variable region binds to the peptide/MHC complex (see FIG. 1). The constant region of the TCR consists of short connecting sequences in which a cysteine residue forms disulfide bonds, which forms a link between the two chains.

The variable domains of both the TCR α-chain and β-chain have three hypervariable or complementarity determining regions (CDRs). The variable region of the β-chain also has an additional area of hypervariability (HV4), however, this does not normally contact antigen and is therefore not considered a CDR.

The TCR also comprises up to five invariant chains γ, δ, ε (collectively termed CD3) and ζ. The CD3 and ζ subunits mediate TCR signalling through specific cytoplasmic domains which interact with second-messenger and adapter molecules following the recognition of the antigen by αβ or γδ. Cell-surface expression of the TCR complex is preceded by the pair-wise assembly of subunits in which both the transmembrane and extracellular domains of TCR α and β and CD3 γ and δ play a role

TCRs are therefore commonly composed of the CD3 complex and the TCR α and β chains, which are in turn composed of variable and constant regions (FIG. 1).

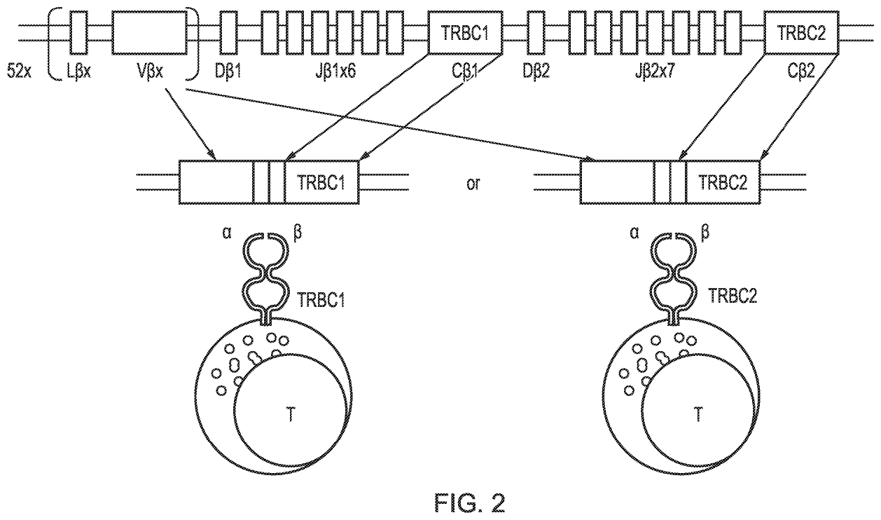

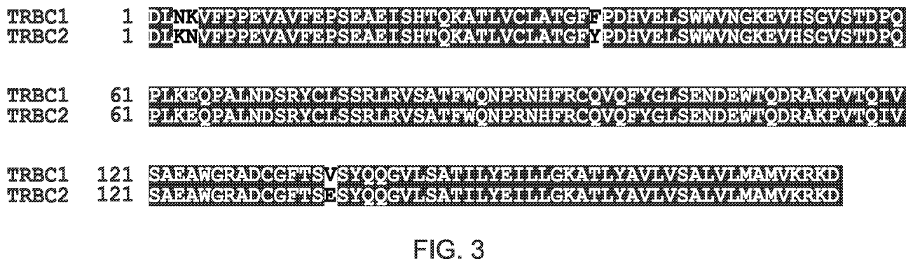

The locus (Chr7:q34) which supplies the TCR β-constant region (TRBC) has duplicated in evolutionary history to produce two almost identical and functionally equivalent genes: TRBC1 and TRBC2 (FIG. 2), which differ by only 4 amino acid in the mature protein produced by each (FIG. 3). Each TCR will comprise, in a mutually exclusive fashion, either TRBC1 or TRBC2 and as such, each αβ T-cell will express either TRBC1 or TRBC2, in a mutually exclusive manner.

Despite the similarity between the sequence of the TRBC1 and TRBC2, it is possible to discriminate between them. The amino acid sequences of TRBC1 and TRBC2 can be discriminated whilst in situ on the surface of a cell, for example a T-cell.

### Antigen Binding Domain

The present disclosure provides a humanised anti-TRBC1 antigen-binding domain which has a variable heavy chain (VH) and a variable light chain (VL) which comprise the following complementarity determining regions (CDRs):

VH CDR1:
(SEQ ID No. 1)
GYTFTGY;
VH CDR2:
(SEQ ID No. 2)
NPYNDD;
VH CDR3:
(SEQ ID No. 3)
GAGYNFDGAYRFFDF;
VL CDR1:
(SEQ ID No. 4)
RSSQRLVHSNGNTYLH;
VL CDR2:
(SEQ ID No. 5)
RVSNRFP;
and
VL CDR3:
(SEQ ID No. 6)
SQSTHVPYT.

The antigen binding domain comprises human framework regions, or human framework regions with one or more mutations. For example, the framework region(s) may comprise one or more substitutions compared to the human framework region sequence. The substitutions may be “back-mutations” where one or more amino acids are substituted with the equivalent residue from the murine antibody sequence. The murine antibody variable heavy chain (VH) sequence is shown below as SEQ ID No. 7 and the variable light chain (VL) sequence shown as SEQ ID No. 8. In both sequences, the CDR sequences are in bold and underlined.

murine Jovi-1 VH
SEQ ID No. 7
EVRLQQSGPDLIKPGASVKMSCKASGYTFTGYVMHWVKQRP
GQGLEWIGFINPYNDDIQSNERFRGKATLTSDKSSTTAYME
LSSLTSEDSAVYYCARGAGYNFDGAYRFFDFWGQGTTLTVS
S
murine Jovi-1 VL
SEQ ID No. 8
DVVMTQSPLSLPVSLGDQASISCRSSQRLVHSNGNTYLHWY
LQKPGQSPKLLIYRVSNRFPGVPDRFSGSGSGTDFTLKISR
VEAEDLGIYFCSQSTHVPYTFGGGTKLEIKR

A humanised VH sequence comprising the murine JOVI-1 CDRs shown as SEQ ID No. 1, 2 and 3 may comprise 6 or fewer, 5 or fewer, 4 or fewer, 3 or fewer, 2 or 1 mutations compared to the wild-type human framework region sequence.

A humanised VL sequence comprising the murine JOVI-1 CDRs shown as SEQ ID No. 4, 5 and 6 may comprise 6 or fewer, 5 or fewer, 4 or fewer, 3 or fewer, 2 or 1 mutation compared to the wild-type human framework region sequence.

The VH sequence may comprise JOVI-1 VH CDRs with the human framework H-AF062256. This sequence is shown as SEQ ID No. 9. The CDR sequences are underlined.

Humanised Jovi-1 H-AF062256 framework
SEQ ID No. 9
QVQLVQSGAEVKKPGASVKVSCKASGYTFTGYVMHWVRQAP
GQGLEWMGFINPYNDDIQSNERFRGRVTMTRDTSISTAYME
LSRLRSDDTAVYYCARGAGYNFDGAYRFFDFWGQGTMVTVS
S

The VH sequence may comprise JOVI-1 VH CDRs with the human framework H-EF177999. This sequence is shown as SEQ ID No. 10. The CDR sequences are underlined.

Humanised Jovi-1 H-EF177999 framework
SEQ ID No. 10
EVQLVESGAEVKKPGASVKVSCKASGYTFTGYVMHWVRQAP
GQGLEWMGFINPYNDDIQSNERFRGRVTMTRDTSTSTVYME
LSSLRSEDTAVYYCARGAGYNFDGAYRFFDFWGQGTLVTVS
S

The VH sequence may comprise JOVI-1 VH CDRs with the human framework H-KF688165. This sequence is shown as SEQ ID No. 11. The CDR sequences are underlined.

Humanised Jovi-1 H-H-KF688165 framework
SEQ ID No. 11
QVQLVQSGAEVKKPGASVKVSCKASEYSFTGYVMHWVRQAP
GQGLEWMGFINPYNDDIQSNERFRGRVTMTRDTSISTAYME
VSSLTSDDAAIYYCARGAGYNFDGAYRFFDFWGQGTLVTVS
S

The VH sequence may comprise the sequence shown as SEQ ID No. 9, 10 or 11 with one or more mutations, such as back-mutations. The VH sequence may comprise 6 or fewer, 5 or fewer, 4 or fewer, 3 or fewer, 2 or 1 mutation compared to the wild-type human framework region sequence. For example, the VH sequence may comprise the sequence shown as SEQ ID No. 9 with one of the sets of back-mutations shown in Table 1 in the Examples.

The VH sequence may comprise one of the sequences shown as SEQ ID No. 12 to 18. The CDR sequences are underlined and back-mutations are shown in bold.

mutation K73
SEQ ID No. 12
QVQLVQSGAEVKKPGASVKVSCKASGYTFTGYVMHWVRQAP
GQGLEWMGFINPYNDDIQSNERFRGRVTMTRDKSISTAYME
LSRLRSDDTAVYYCARGAGYNFDGAYRFFDFWGQGTMVTVS
S
mutation S71
SEQ ID No. 13
QVQLVQSGAEVKKPGASVKVSCKASGYTFTGYVMHWVRQAP
GQGLEWMGFINPYNDDIQSNERFRGRVTMTSDTSISTAYME
LSRLRSDDTAVYYCARGAGYNFDGAYRFFDFWGQGTMVTVS
S
mutations S71, K73
SEQ ID No. 14
QVQLVQSGAEVKKPGASVKVSCKASGYTFTGYVMHWVRQAP
GQGLEWMGFINPYNDDIQSNERFRGRVTMTSDKSISTAYME
LSRLRSDDTAVYYCARGAGYNFDGAYRFFDFWGQGTMVTVS
S
mutation I48
SEQ ID No. 15
QVQLVQSGAEVKKPGASVKVSCKASGYTFTGYVMHWVRQAP
GQGLEWIGFINPYNDDIQSNERFRGRVTMTRDTSISTAYME
LSRLRSDDTAVYYCARGAGYNFDGAYRFFDFWGQGTMVTVS
S
mutation I48, K73
SEQ ID No. 16
QVQLVQSGAEVKKPGASVKVSCKASGYTFTGYVMHWVRQAP
GQGLEWIGFINPYNDDIQSNERFRGRVTMTRDKSISTAYME
LSRLRSDDTAVYYCARGAGYNFDGAYRFFDFWGQGTMVTVS
S
mutations M20, S71, K73
SEQ ID No. 17
QVQLVQSGAEVKKPGASVKMSCKASGYTFTGYVMHWVRQAP
GQGLEWMGFINPYNDDIQSNERFRGRVTMTSDKSISTAYME
LSRLRSDDTAVYYCARGAGYNFDGAYRFFDFWGQGTMVTVS
S
mutations M20, I48
SEQ ID No. 18
QVQLVQSGAEVKKPGASVKMSCKASGYTFTGYVMHWVRQAP
GQGLEWIGFINPYNDDIQSNERFRGRVTMTRDTSISTAYME
LSRLRSDDTAVYYCARGAGYNFDGAYRFFDFWGQGTMVTVS
S

The VL sequence may comprise JOVI-1 VL CDRs with the human framework 3aaz. This sequence is shown as SEQ ID No. 19. The CDR sequences are underlined.

SEQ ID No. 19
DIVMTQSPLSLPVTPGEPASISCRSSQRLVHSNGNTYLHWY
LQKPGQSPRLLIYRVSNRFPGVPDRFSGSGSGTDFTLKISR
VEAEDVGVYYCSQSTHVPYTFGQGTKLEIK

The VL sequence may comprise the sequence shown as SEQ ID No. 19 with one or more mutations, such as back-mutations. The VL sequence may comprise 6 or fewer, 5 or fewer, 4 or fewer, 3 or fewer, 2 or 1 mutation compared to the wild-type human framework region sequence. For example, the VL sequence may comprise one of the sequences shown as SEQ ID No. 20 to 34. The CDR sequences are underlined and back-mutations are shown in bold.

SEQ ID No. 20
DIVMTQSPLSLPVTLGEQASISCRSSQRLVHSNGNTYLHWYLQKP
GQSPRLLIYRVSNRFPGVPDRFSGSGSGTDFTLKISRVEAEDVGV
YFCSQSTHVPYTFGGGTKLEIK
SEQ ID No. 21
DIVMTQSPLSLPVTLGEQASISCRSSQRLVHSNGNTYLHWYLQKP
GQSPRLLIYRVSNRFPGVPDRFSGSGSGTDFTLKISRVEAEDVGV
YFCSQSTHVPYTFGQGTKLEIK
SEQ ID No. 22
DIVMTQSPLSLPVTLGEQASISCRSSQRLVHSNGNTYLHWYLQKP
GQSPRLLIYRVSNRFPGVPDRFSGSGSGTDFTLKISRVEAEDVGV
YYCSQSTHVPYTFGGGTKLEIK
SEQ ID No. 23
DIVMTQSPLSLPVTLGEQASISCRSSQRLVHSNGNTYLHWYLQKP
GQSPRLLIYRVSNRFPGVPDRFSGSGSGTDFTLKISRVEAEDVGV
YYCSQSTHVPYTFGQGTKLEIK
SEQ ID No. 24
DIVMTQSPLSLPVTLGEPASISCRSSQRLVHSNGNTYLHWYLQKP
GQSPRLLIYRVSNRFPGVPDRFSGSGSGTDFTLKISRVEAEDVGV
YFCSQSTHVPYTFGGGTKLEIK
SEQ ID No. 25
DIVMTQSPLSLPVTLGEPASISCRSSQRLVHSNGNTYLHWYLQKP
GQSPRLLIYRVSNRFPGVPDRFSGSGSGTDFTLKISRVEAEDVGV
FYCSQSTHVPYTFGQGTKLEIK
SEQ ID No. 26
DIVMTQSPLSLPVTLGEPASISCRSSQRLVHSNGNTYLHWYLQKP
GQSPRLLIYRVSNRFPGVPDRFSGSGSGTDFTLKISRVEAEDVGV
YYCSQSTHVPYTFGGGTKLEIK
SEQ ID No. 27
DIVMTQSPLSLPVTLGEPASISCRSSQRLVHSNGNTYLHWYLQKP
GQSPRLLIYRVSNRFPGVPDRFSGSGSGTDFTLKISRVEAEDVGV
YYCSQSTHVPYTFGQGTKLEIK
SEQ ID No. 28
DIVMTQSPLSLPVTPGEQASISCRSSQRLVHSNGNTYLHWYLQKP
GQSPRLLIYRVSNRFPGVPDRFSGSGSGTDFTLKISRVEAEDVGV
YFCSQSTHVPYTFGGGTKLEIK
SEQ ID No. 29
DIVMTQSPLSLPVTPGEQASISCRSSQRLVHSNGNTYLHWYLQKP
GQSPRLLIYRVSNRFPGVPDRFSGSGSGTDFTLKISRVEAEDVGV
YFCSQSTHVPYTFGQGTKLEIK
SEQ ID No. 30
DIVMTQSPLSLPVTPGEQASISCRSSQRLVHSNGNTYLHWYLQKP
GQSPRLLIYRVSNRFPGVPDRFSGSGSGTDFTLKISRVEAEDVGV
YYCSQSTHVPYTFGGGTKLEIK
SEQ ID No. 31
DIVMTQSPLSLPVTPGEQASISCRSSQRLVHSNGNTYLHWYLQKP
GQSPRLLIYRVSNRFPGVPDRFSGSGSGTDFTLKISRVEAEDVGV
YYCSQSTHVPYTFGQGTKLEIK
SEQ ID No. 32
DIVMTQSPLSLPVTPGEPASISCRSSQRLVHSNGNTYLHWYLQKP
GQSPRLLIYRVSNRFPGVPDRFSGSGSGTDFTLKISRVEAEDVGV
YFCSQSTHVPYTFGGGTKLEIK
SEQ ID No. 33
DIVMTQSPLSLPVTPGEPASISCRSSQRLVHSNGNTYLHWYLQKP
GQSPRLLIYRVSNRFPGVPDRFSGSGSGTDFTLKISRVEAEDVGV
YFCSQSTHVPYTFGQGTKLEIK
SEQ ID No. 34
DIVMTQSPLSLPVTPGEPASISCRSSQRLVHSNGNTYLHWYLQKP
GQSPRLLIYRVSNRFPGVPDRFSGSGSGTDFTLKISRVEAEDVGV
YYCSQSTHVPYTFGGGTKLEIK

The anti-TRBC1 antigen binding domain may comprise:
a) a VH domain which comprises JOVI-1 VH CDRs with the human framework H-AF062256 or a variant thereof; and
b)) a VH domain which comprises JOVI-1 VL CDRs with the human framework 3aaz or a variant thereof.

The variant may have 5 or fewer, 4 or fewer, 3 or fewer, 2 or 1 mutation compared to the wild-type human framework region sequence.

The VH domain may comprise the sequence shown as SEQ ID No. 9, SEQ ID No. 12, SEQ ID No. 13, SEQ ID No. 14, SEQ ID No. 15, SEQ ID No. 16, SEQ ID No. 17 or SEQ ID No. 18. The VL domain may comprise the sequence shown as SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 29, SEQ ID No. 30, SEQ ID No. 31, SEQ ID No. 32, SEQ ID No. 33, SEQ ID No. 34.

The anti-TRBC1 antigen binding domain may comprise:
a) a VH domain which comprises the sequence shown as SEQ ID No 9; and
b) a VH domain which comprises the sequence shown as SEQ ID No. 19.

### Antibody

The antigen-binding domain of the first aspect of the disclosure may be an antibody or a functional fragment thereof. The antibody may be a therapeutic antibody, such as a depleting antibody. The antibody may be a bispecific antibody which binds TRBC1 together with another antigen. The antibody may, for example, be a dual affinity re-targeting antibody.

The term ‘depleting antibody’ is used in the conventional sense to relate to an antibody which binds to an antigen (i.e. TRBC1) present on a target T-cell and mediates death of the target T-cell. The administration of a depleting antibody to a subject therefore results in a reduction/decrease in the number of cells within the subject which express the target antigen.

As used herein, “antibody” means a polypeptide having an antigen binding site which comprises at least one complementarity determining region CDR. The antibody may comprise 3 CDRs and have an antigen binding site which is equivalent to that of a domain antibody (dAb). The antibody may comprise 6 CDRs and have an antigen binding site which is equivalent to that of a classical antibody molecule. The remainder of the polypeptide may be any sequence which provides a suitable scaffold for the antigen binding site and displays it in an appropriate manner for it to bind the antigen.

The antibody may be a whole immunoglobulin molecule or a part thereof such as a Fab, F(ab)′2, Fv, single chain Fv (ScFv) fragment, and scFv-Fc fusion or diabody, triabody or nanobody which retains the antigen specificity of the full antibody. The antibody may be a bifunctional antibody. The antibody may be non-human, chimeric, humanised or fully human.

### Conjugates

The antibody may be a conjugate of the antibody and another agent or antibody, for example the conjugate may be a detectable entity or a chemotherapeutic entity.

The detectable entity may be a fluorescent moiety, for example a fluorescent peptide. A “fluorescent peptide” refers to a polypeptide which, following excitation, emits light at a detectable wavelength. Examples of fluorescent proteins include, but are not limited to, fluorescein isothiocyanate (FITC), phycoerythrin (PE), allophycocyanin (APC), green fluorescent protein (GFP), enhanced GFP, red fluorescent protein (RFP), blue fluorescent protein (BFP) and mCherry.

A chemotherapeutic entity as used herein refers to an entity which is destructive to a cell, that is the entity reduces the viability of the cell. The chemotherapeutic entity may be a cytotoxic drug. A chemotherapeutic agent contemplated includes, without limitation, alkylating agents, nitrosoureas, ethylenimines/methylmelamine, alkyl sulfonates, antimetabolites, pyrimidine analogs, epipodophylotoxins, enzymes such as L-asparaginase; biological response modifiers such as IFNα, IL-2, G-CSF and GM-CSF; platinum coordination complexes such as cisplatin and carboplatin, anthracenediones, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

A TRBC1-specific antibody-drug conjugate enables the targeted delivery of a chemotherapeutic entity to cells which express TRBC1.

### Immune Cell Engagers

Immune cell engager molecules are a class of antibody-type molecules that have been developed, primarily for the use as anti-cancer drugs. They direct immune effector cells of a host's immune system against a target cell, such as a cancer cell. In these immune cell engager molecules, at least one binding domain binds to the immune cell via, for example, a receptor expressed on the immune cell, and another binding domain binds to a target cell such as a tumour cell (e.g. via a tumour specific molecule). Since the immune cell engager molecule binds both the target cell and the immune cell, it brings the target cell into proximity with the immune cell, so that the immune cell can exert its effect, for example, a cytotoxic effect on a cancer cell. The formation of the immune cell: immune cell engager:cancer cell complex induces signalling in the immune cell leading to, for example, the release of cytotoxic mediators. Ideally, the agent only induces the desired signalling in the presence of the target cell, leading to selective killing.

Thus, an immune cell engager molecule which of the present disclosure brings a TRBC1-expressing cell (for example, a TRBC1+ cancer cell) into proximity with an immune cell, so that the immune cell can exert its effect on the cancer cell. The requirement of co-localisation via binding of the TRBC1 immune cell engager molecule may lead to selective killing of TRBC1-positive cells. Suitably, an immune cell engager molecule of the present disclosure is able to activate an immune cell following binding of the immune cell engager molecule to TRBC1 expressed on the surface of target cells.

The immune cell engager may be multivalent and may comprise multiple copies of the same immune cell binding domain.

The immune cell engager may comprise multiple immune cell binding domains wherein each immune cell binding domain has a different target molecule expressed on the immune cell.

The immune cell engager may be a T cell engager, an NK cell engager, a B cell engager, a dendritic cell engager, or a macrophage cell engager. In some embodiments, the immune cell engager binds to and activates an immune cell, e.g., an effector cell. In some embodiments, the immune cell engager binds to, but does not activate, an immune cell, e.g., an effector cell.

The immune cell engager may be capable of binding, for example, a T cell (i.e. an alpha beta T cell), an NKT cell, a gamma delta T cell or an NK cell.

### Bi-Specific T Cell Engagers (Bites) and Tri-Specific T Cell Engagers

The present disclosure provides a T cell engager molecule which is a bi-specific T cell engager (BiTE) which comprises a humanised anti-TRBC1 antigen-binding domain as described herein as a first domain, and a T cell activating domain as a second domain. A T cell activating domain is a domain capable of activating a T cell.

Bi-specific T cell engaging molecules typically comprise a binding domain which binds to a T cell via, for example, the CD3 receptor, and the other to a target cell such as a tumour cell (e.g. via a tumour specific molecule). Since the bi-specific molecule binds both the target cell and the T cell, it brings the target cell into proximity with the T cell, so that the T cell can exert its effect, for example, a cytotoxic effect on a cancer cell. The formation of the T cell:bi-specific Ab:cancer cell complex induces signalling in the T cell leading to, for example, the release of cytotoxic mediators. Ideally, the agent only induces the desired signalling in the presence of the target cell, leading to selective killing.

Thus, a T cell engager molecule which is a bi-specific molecule of the present invention brings a TRBC1-expressing cell (for example, a TRBC1+ cancer cell) into proximity with a T cell, so that the T cell can exert its effect on the cancer cell. The requirement of co-localisation via binding of the TRBC1 bi-specific molecule suitably leads to selective killing of TRBC1-positive cells. In other words, a bi-specific molecule of the present invention is able to activate T cells following binding of the bi-specific molecule to TRBC1 expressed on the surface of target cells.

BiTEs are commonly made by fusing an anti-CD3 scFv to an anti-target antigen scFv via a short five residue peptide linker (e.g. GGGGS (SEQ ID NO: 36)).

In one embodiment, the present invention provides a T cell engager molecule which is a tri-specific T cell engager which comprises a humanised anti-TRBC1 antigen-binding domain as described herein as a first domain, a T cell activating domain as a second domain, and another T cell co-activating domain as a third domain. A T cell activating domain is a domain capable of activating a T cell.

A tri-specific T cell engager molecule may comprise the same first and second domains described herein for a bi-specific T cell engager molecule, with the addition of a third domain which is also capable of co-activating a T cell. Accordingly, a tri-specific T cell engager molecule may have a similar effect and function as a bi-specific T cell engager molecule as described herein.

### Second Domain—T Cell Activating Domain

The second domain of the bi-specific T cell engager molecule of the present disclosure or tri-specific T cell engager molecule of the present disclosure is capable of activating T cells. T cells have a T cell-receptor (TCR) at the cell surface which recognises antigenic peptides when presented by an MHC molecule on the surface of an antigen presenting cell. Such antigen recognition results in the phosphorylation of immunoreceptor tyrosine-based activation motifs (ITAMs) by Src family kinases, triggering recruitment of further kinases which results in T cell activation including Ca²⁺ release. The second domain may cause T cell activation by triggering the same pathway triggered by antigen-specific recognition by the TCR. Thus, the second domain may induce T cell signalling and result in T cell activation.

Due to its central role in modulating T cell activity, there have been attempts to develop molecules that are capable of binding TCR/CD3. Much of this work has focused on the generation of antibodies that are specific for the human CD3 antigen.

The second domain of the bi-specific T cell engager molecule of the disclosure or tri-specific T cell engager molecule of the disclosure may bind CD3.

The second domain may comprise an antibody or part thereof which specifically binds CD3, such as OKT3, WT32, anti-leu-4, UCHT-1, SPV-3TA, TR66, SPV-T3B or affinity tuned variants thereof.

Alternatively, the second domain may comprise a CD3-binding molecule which is not derived from or based on an immunoglobulin. A number of “antibody mimetic” designed repeat proteins (DRPs) have been developed to exploit the binding abilities of non-antibody polypeptides. Such molecules include ankyrin or leucine-rich repeat proteins e.g. DARPins (Designed Ankyrin Repeat Proteins), Anticalins, Avimers and Versabodies.

The second domain of the bi-specific T cell engager molecule of the disclosure or tri-specific T cell engager molecule of the disclosure may comprise all or part of the monoclonal antibody OKT3, which was the first monoclonal antibody approved by the FDA. OKT3 is available from ATCC CRL 8001. The antibody sequences are published in U.S. Pat. No. 7,381,803.

### Third Domain—CD28 Binding

The third domain of the tri-specific T cell engager molecule of the disclosure may bind CD28.

The third domain may comprise an antibody or part thereof which specifically binds CD28, such as TGN1412 or affinity tuned variants thereof.

The third domain of the tri-specific T cell engager molecule of the disclosure may comprise all or part of the monoclonal antibody TGN1412.

### Gamma Delta Cell Engagers

The immune cell engager of the present disclosure may be a molecule that engages a gamma delta T cell. Examples of gamma delta cells include Vγ9Vδ2 T cells.

The second domain of the gamma delta T cell engager may bind CD3 as described herein.

The second domain of the gamma delta T cell engager may bind to the Vγ9 chain of the Vγ9Vδ2+ γδ T cell receptor. Suitably antibodies capable of binding to Vγ9Vδ2 T cells are described in WO2020/159368, for example.

### NKT Cell Engagers

An immune cell engager of the present disclosure may be a molecule that engages a NKT cell.

The second domain of the NKT cell engager may bind CD3 as described herein.

### Bi-Specific Killer Cell Engagers (Bikes) and Tri-Specific Killer Cell Engagers (Trikes)

The present disclosure provides a NK cell engager molecule which is a bi-specific killer cell engager molecule (BiKE) which comprises a humanised anti-TRBC1 antigen-binding domain as described herein as a first domain, and a NK cell activating domain as a second domain. A NK cell activating domain is a domain capable of activating a NK cell.

The BiKE or TRiKE may comprise multiple copies of the second domain, and thus may encompass multiple copies of a NK cell activating domain. The BiKE or TRiKE may comprise 1, 2, 3, 4, 5 or more of the same NK cell activating domain. For example, the NK cell engager may comprise three CD16-binding domains which each bind to CD16.

Bi-specific killer cell engaging molecules are a class of bi-specific antibody-type molecules that have been developed, primarily for the use as anti-cancer drugs. They direct a host's immune system, more specifically the NK cells' cytotoxic activity, against a target cell, such as a cancer cell. In these bi-specific killer cell engager molecules, one binding domain binds to a NK cell via, for example, the CD16 receptor, and the other to a target cell such as a tumour cell (e.g. via a tumour specific molecule). Since the bi-specific killer cell engager molecule binds both the target cell and the NK cell, it brings the target cell into proximity with the NK cell, so that the NK cell can exert its effect, for example, a cytotoxic effect on a cancer cell. The formation of the NK cell:bi-specific Ab:cancer cell complex induces signalling in the NK cell leading to, for example, the release of cytotoxic mediators. Ideally, the agent only induces the desired signalling in the presence of the target cell, leading to selective killing.

Thus, a NK cell engager of the present disclosure brings a TRBC1-expressing cell (for example, a TRBC1+ cancer cell) into proximity with a NK cell, so that the NK cell can exert its effect on the cancer cell. The requirement of co-localisation via binding of the TRBC1 bi-specific killer cell engager molecule leads to selective killing of TRBC1-positive cells. In other words, a NK cell engager molecule of the present invention is able to activate NK cells following binding of the NK cell engager molecule to TRBC1 expressed on the surface of target cells.

Suitably, the second domain activates a NK cell by binding CD16 on the NK cell surface.

Suitably, the second domain comprises a CD16-specific antibody or part thereof.

The second domain may comprise an antibody or part thereof which specifically binds CD16, such as 3G8 or LSIV21 or affinity tuned variants thereof.

The immune cell engagers described herein may comprise a signal peptide to aid in their production. The signal peptide may cause the molecule to be secreted by a host cell, such that the immune cell engager can be harvested from the host cell supernatant.

The signal peptide may be at the amino terminus of the molecule. The bi-specific molecule may have the general formula: Signal peptide-first domain-second domain.

The bi-specific molecule may comprise a spacer sequence to connect the first domain with the second domain and spatially separate the two domains.

The spacer sequence may, for example, comprise an IgG1 hinge or a CD8 stalk. The linker may alternatively comprise an alternative linker sequence which has similar length and/or domain spacing properties as an IgG1 hinge or a CD8 stalk. Alternatively, a short five residue peptide linker (e.g. GGGGS (SEQ ID NO: 36)) may also be used.

### Chimeric Antigen Receptor (Car)

The present disclosure provides a CAR which selectively recognises TRBC1.

Chimeric antigen receptors (CARs), also known as chimeric T-cell receptors, artificial T-cell receptors and chimeric immunoreceptors, are engineered receptors, which graft an arbitrary specificity onto an immune effector cell. In a classical CAR, the specificity of a monoclonal antibody is grafted on to a T-cell. CAR-encoding nucleic acids may be transferred to T-cells using, for example, retroviral vectors. In this way, a large number of cancer-specific T-cells can be generated for adoptive cell transfer. Phase I clinical studies of this approach show efficacy.

The target-antigen binding domain of a CAR is commonly fused via a spacer and transmembrane domain to an endodomain, which comprises or associates with an intracellular T-cell signalling domain. When the CAR binds the target-antigen, this results in the transmission of an activating signal to the T-cell it is expressed on.

The CAR may also comprise a transmembrane domain which spans the membrane. It may comprise a hydrophobic alpha helix. The transmembrane domain may be derived from CD28, which gives good receptor stability.

The endodomain is the portion of the CAR involved in signal-transmission. The endodomain either comprises or associates with an intracellular T-cell signalling domain. After antigen recognition, receptors cluster and a signal is transmitted to the cell. The most commonly used T-cell signalling component is that of CD3-zeta which contains 3 ITAMs. This transmits an activation signal to the T-cell after antigen is bound. CD3-zeta may not provide a fully competent activation signal and additional co-stimulatory signaling may be needed. For example, chimeric CD28 and OX40 can be used with CD3-Zeta to transmit a proliferative/survival signal, or all three can be used together.

The endodomain of the CAR may comprise the CD28 endodomain and OX40 and CD3-Zeta endodomain.

Alternatively, the CAR of the second aspect of the disclosure may lack an intracellular signalling domain but may be capable of associating with a separate molecule which provides the signalling functionality.

CAR signalling systems have previously been described which comprise two parts: a CAR, which comprises the antigen binding domain and a transmembrane domain; and an intracellular signalling component which comprises an intracellular signalling domain. One or more co-stimulatory domains may be located on the CAR and/or the intracellular signalling component.

Heterodimerisation between the CAR and the intracellular signalling component produces a functional CAR system. Heterodimerisation may occur spontaneously, as described in WO2016/124930; or it may occur only in the presence of a chemical inducer of dimerization (CID), as described in WO2015/150771. In a third alternative, heterodimerization is disrupted by the presence of an agent, such as a particular small molecule, so CAR-mediated signalling only occurs in the absence of the agent. Such a system is described in WO2016/030691.

The CAR may comprise a signal peptide so that when the CAR is expressed inside a cell, such as a T-cell, the nascent protein is directed to the endoplasmic reticulum and subsequently to the cell surface, where it is expressed.

The CAR may comprise a spacer sequence to connect the TRBC-binding domain with the transmembrane domain and spatially separate the TRBC-binding domain from the membrane. A flexible spacer allows to the TRBC-binding domain to orient in different directions to enable TRBC binding.

The spacer sequence may, for example, comprise an IgG1 Fc region, an IgG1 hinge or a CD8 stalk, or a combination thereof.

### Nucleic Acid

The present disclosure further provides a nucleic acid encoding a BiTE or CAR as defined above.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the disclosure may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

The present disclosure also provides a nucleic acid construct which comprises a first nucleic acid encoding a CAR as defined above; and a second nucleic acid encoding a suicide gene.

Suitable suicide genes for use in a CAR-expressing cell of the disclosure include RQR8, which is described in WO2013/153391; and RapCasp9, which is described in WO2016/135470.

In the nucleic acid construct described above, the first and second nucleic acid sequences may be in either order.

### Vector

The present disclosure also provides a vector, or kit of vectors, which comprises one or more nucleic acid sequence(s) or nucleic acid construct(s) of the disclosure. Such a vector may be used to introduce the nucleic acid sequence(s) or construct(s) into a host cell, for example, so that it expresses a CAR having an antigen-binding domain according to the first aspect of the disclosure.

The vector may, for example, be a plasmid or a viral vector, such as a retroviral vector or a lentiviral vector, or a transposon-based vector or synthetic mRNA.

The vector may be capable of transfecting or transducing a T cell or a NK cell.

### Cell

The present disclosure also relates to a cell, such as an immune cell, comprising a CAR according to the first aspect of the disclosure.

The cell may comprise a nucleic acid, a nucleic acid construct or a vector of the present disclosure.

The cell may be a T-cell or a natural killer (NK) cell.

T cell may be T cells or T lymphocytes which are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognise their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr cells or Th3 cells) may originate during a normal immune response.

The cell may be a Natural Killer cell (or NK cell). NK cells form part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The CAR cells of the disclosure may be any of the cell types mentioned above.

T or NK cells expressing a CAR according to the first aspect of the disclosure may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, T or NK cells expressing a CAR according to the first aspect of the disclosure may be derived from ex vivo differentiation of inducible progenitor cells or embryonic progenitor cells to T or NK cells. Alternatively, an immortalized T-cell line which retains its lytic function and could act as a therapeutic may be used.

In all these embodiments, CAR cells are generated by introducing DNA or RNA coding for the CAR by one of many means including transduction with a viral vector, transfection with DNA or RNA.

The CAR-expressing cell of the disclosure may be an ex vivo T or NK cell from a subject. The T or NK cell may be from a peripheral blood mononuclear cell (PBMC) sample. T or NK cells may be activated and/or expanded prior to being transduced with nucleic acid encoding a CAR according to the first aspect of the disclosure, for example by treatment with an anti-CD3 monoclonal antibody.

The T or NK cell of the disclosure may be made by:
(i) isolation of a T or NK cell-containing sample from a subject or other sources listed above; and
(ii) transduction or transfection of the T or NK cells with a nucleic acid sequence(s) encoding a CAR of the disclosure.

The T or NK cells may then by purified, for example, selected on the basis of expression of the antigen-binding domain of the antigen-binding polypeptide.

The present disclosure also provides a kit which comprises a T or NK cell comprising a CAR according to the first aspect of the disclosure.

### Pharmaceutical Composition

The present disclosure also relates to a pharmaceutical composition containing a therapeutic entity such as a CAR-expressing cell, a therapeutic antibody or conjugate thereof, or a bi-specific T-cell engager of the present disclosure. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

### T-Cell Malignancies

The present disclosure relates to agents, cells and methods for treating a T-cell lymphoma and/or leukaemia.

A method for treating a T-cell lymphoma and/or leukaemia relates to the therapeutic use of an agent. Herein the agent may be administered to a subject having an existing disease of T-cell lymphoma and/or leukaemia in order to lessen, reduce or improve at least one symptom associated with the disease and/or to slow down, reduce or block the progression of the disease.

The method of the present disclosure may be used for the treatment of any malignancy associated with the clonal expansion of a cell expressing a T-cell receptor (TCR) comprising TRBC1, such as a T-cell lymphoma and/or leukaemia.

The present disclosure provides a method for treating a TRBC1-positive malignancy in a patient comprising administering to the patient an autologous anti-TRBC1 CAR T-cell or a population of autologous anti-TRBC1 CAR T-cells.

The present disclosure provides an autologous anti-TRBC1 CAR T-cell or a population of autologous anti-TRBC1 CAR T-cells for use in treating a TRBC1-positive malignancy.

The present disclosure provides the use of an autologous anti-TRBC1 CAR T-cell or a population of autologous anti-TRBC1 CAR T-cells in the manufacture of a medicament from treating a TRBC1-positive malignancy.

The method of the present disclosure may be used to treat a T-cell lymphoma in which the malignant T-cell expresses a TCR comprising TRBC1. ‘Lymphoma’ is used herein according to its standard meaning to refer to a cancer which typically develops in the lymph nodes, but may also affect the spleen, bone marrow, blood and other organs.

Lymphoma typically presents as a solid tumour of lymphoid cells. The primary symptom associated with lymphoma is lymphadenopathy, although secondary (B) symptoms can include fever, night sweats, weight loss, loss of appetite, fatigue, respiratory distress and itching.

The method of the present disclosure may be used to treat a T-cell leukaemia in which the malignant T-cell expresses a TCR comprising TRBC1. ‘Leukaemia’ is used herein according to its standard meaning to refer to a cancer of the blood or bone marrow.

The following is an illustrative, non-exhaustive list of diseases which may be treated by the method of the present disclosure.

### Peripheral T-Cell Lymphoma

Peripheral T-cell lymphomas are relatively uncommon lymphomas and account fewer than 10% of all non-Hodgkin lymphomas (NHL). However, they are associated with an aggressive clinical course and the causes and precise cellular origins of most T-cell lymphomas are still not well defined.

Lymphoma usually first presents as swelling in the neck, underarm or groin. Additional swelling may occur where other lymph nodes are located such as in the spleen. In general, enlarged lymph nodes can encroach on the space of blood vessels, nerves, or the stomach, leading to swollen arms and legs, to tingling and numbness, or to feelings of being full, respectively. Lymphoma symptoms also include nonspecific symptoms such as fever, chills, unexplained weight loss, night sweats, lethargy, and itching.

The WHO classification utilizes morphologic and immunophenotypic features in conjunction with clinical aspects and in some instances genetics to delineate a prognostically and therapeutically meaningful categorization for peripheral T-cell lymphomas (Swerdlow et al.; WHO classification of tumours of haematopoietic and lymphoid tissues. 4th ed.; Lyon: IARC Press; 2008). The anatomic localization of neoplastic T-cells parallels in part their proposed normal cellular counterparts and functions and as such T-cell lymphomas are associated with lymph nodes and peripheral blood. This approach allows for better understanding of some of the manifestations of the T-cell lymphomas, including their cellular distribution, some aspects of morphology and even associated clinical findings.

The most common of the T-cell lymphomas is peripheral T-cell lymphoma, not otherwise specified (PTCL-NOS) comprising 25% overall, followed by angioimmunoblastic T-cell lymphoma (AITL) (18.5%)

### Peripheral T-Cell Lymphoma, not Otherwise Specified (PTCL-NOS)

PTCL-NOS comprises over 25% of all peripheral T-cell lymphomas and NK/T-cell lymphomas and is the most common subtype. It is determined by a diagnosis of exclusion, not corresponding to any of the specific mature T-cell lymphoma entities listed in the current WHO 2008. As such it is analogous to diffuse large B-cell lymphoma, not otherwise specified (DLBCL-NOS).

Most patients are adults with a median age of 50 and a male to female ratio 2:1. The majority of cases are nodal in origin, however, extranodal presentations occur in approximately 13% of patients and most commonly involve skin and gastrointestinal tract.

The cytologic spectrum is very broad, ranging from polymorphous to monomorphous. Three morphologically defined variants have been described, including lymphoepithelioid (Lennert) variant, T-zone variant and follicular variant. The lymphoepithelioid variant of PTCL contains abundant background epithelioid histiocytes and is commonly positive for CD8. It has been associated with a better prognosis. The follicular variant of PTCL-NOS is emerging as a potentially distinct clinicopathologic entity.

The majority of PTCL-NOS have a mature T-cell phenotype and most cases are CD4-positive. 75% of cases show variable loss of at least one pan T-cell marker (CD3, CD2, CD5 or CD7), with CD7 and CD5 being most often downregulated. CD30 and rarely CD15 can be expressed, with CD15 being an adverse prognostic feature. CD56 expression, although uncommon, also has negative prognostic impact. Additional adverse pathologic prognostic factors include a proliferation rate greater than 25% based on KI-67 expression, and presence of more than 70% transformed cells. Immunophenotypic analysis of these lymphomas has offered little insight into their biology.

### Angioimmunoblastic T-Cell Lymphoma (AITL)

AITL is a systemic disease characterized by a polymorphous infiltrate involving lymph nodes, prominent high endothelial venules (HEV) and peri-vascular expansion of follicular dendritic cell (FDC) meshworks. AITL is considered as a de-novo T-cell lymphoma derived from αβ T-cells of follicular helper type (TFH), normally found in the germinal centres.

AITL is the second most common entity among peripheral T-cell lymphoma and NK/T-cell lymphomas, comprising about 185% of cases. It occurs in middle aged to elderly adults, with a median age of 65 years old, and an approximately equal incidence in males and females. Clinically, patients usually have advanced stage disease, with generalized lymphadenopathy, hepatosplenomegaly and prominent constitutional symptoms. Skin rash with associated pruritus is commonly present. There is often polyclonal hypergammaglobulinemia, associated with autoimmune phenomena.

Three different morphologic patterns are described in AITL The early lesion of AITL (Pattern I) usually shows preserved architecture with characteristic hyperplastic follicles. The neoplastic proliferation is localized to the periphery of the follicles. In Pattern II the nodal architecture is partially effaced with retention of few regressed follicles. The subcapsular sinuses are preserved and even dilated. The paracortex contains arborizing HEV and there is a proliferation of FDC beyond the B-cell follicle. The neoplastic cells are small to medium in size, with minimal cytologic atypia. They often have clear to pale cytoplasm and may show distinct T-cell membranes. A polymorphous inflammatory background is usually evident.

Although AITL is a T-cell malignancy, there is a characteristic expansion of B-cells and plasma cells, which likely reflects the function of the neoplastic cells as TFH cells. Both EBV-positive and EBV-negative B-cells are present. Occasionally, the atypical B-cells may resemble Hodgkin/Reed-Sternberg-like cells morphologically and immunophenotypically, sometimes leading to a diagnostic confusion with that entity.

The B-cell proliferation in AITL may be extensive and some patients develop secondary EBV-positive diffuse large B-cell lymphomas (DLBCL) or—more rarely—EBV-negative B-cell tumors, often with plasmacytic differentiation.

The neoplastic CD4-positive T-cells of AITL show strong expression of CD10 and CD279 (PD-1) and are positive for CXCL13. CXCL13 leads to an increased B-cell recruitment to lymph nodes via adherence to the HEV, B-cell activation, plasmacytic differentiation and expansion of the FDC meshworks, all contributing to the morphologic and clinical features of AITL. Intense PD-1-expression in the perifollicular tumor cells is particularly helpful in distinguishing AITL Pattern I from reactive follicular and paracortical hyperplasia.

The follicular variant of PTCL-NOS is another entity with a TFH phenotype. In contradistinction to AITL, it does not have prominent HEV or extra-follicular expansion of FDC meshworks. The neoplastic cells may form intrafollicular aggregates, mimicking B-cell follicular lymphoma, but also can have interfollicular growth pattern or involve expanded mantle zones. Clinically, the follicular variant of PTCL-NOS is distinct from AITL as patients more often present with early stage disease with partial lymph node involvement and may lack the constitutional symptoms associated with AITL.

### Anaplastic Large Cell Lymphoma (ALCL)

ALCL may be subdivided as ALCL-‘anaplastic lymphoma kinase’ (ALK)+ or ALCL-ALK−.

ALCL-ALK+ is one of the best-defined entities within the peripheral T-cell lymphomas, with characteristic “hallmark cells” bearing horseshoe-shaped nuclei and expressing ALK and CD30. It accounts for about 7% of all peripheral T-cell and NK-cell lymphomas and is most common in the first three decades of life. Patients often present with lymphadenopathy, but the involvement of extranodal sites (skin, bone, soft tissues, lung, liver) and B symptoms is common.

ALCL, ALK+ shows a wide morphologic spectrum, with 5 different patterns described, but all variants contain some hallmark cells. Hallmark cells have eccentric horseshoe- or kidney-shaped nuclei, and a prominent perinuclear eosinophilic Golgi region. The tumour cells grow in a cohesive pattern with predilection for sinus involvement. Smaller tumour cells predominate in the small cell variant, and in the lymphohistiocytic variant abundant histiocytes mask the presence of tumour cells, many of which are small.

By definition, all cases show ALK and CD30 positivity, with expression usually weaker in the smaller tumour cells. There is often loss of pan-T-cell markers, with 75% of cases lacking surface expression of CD3.

ALK expression is a result of a characteristic recurrent genetic alteration consisting of a rearrangement of ALK gene on chromosome 2p23 to one of the many partner genes, resulting in an expression of chimeric protein. The most common partner gene, occurring in 75% of cases, is Nucleophosmin (NPM1) on chromosome 5q35, resulting in t(2;5)(p23;q35). The cellular distribution of ALK in different translocation variants may vary depending on the partner gene.

ALCL-ALK− is included as a provisional category in the 2008 WHO classification. It is defined as a CD30 positive T-cell lymphoma that is morphologically indistinguishable from ALCL-ALK+ with a cohesive growth pattern and presence of hallmark cells, but lacking ALK protein expression.

Patients are usually adults between the ages of 40 and 65, in contrast to ALCL-ALK+, which is more common in children and young adults. ALCL-ALK− can involve both lymph nodes and extranodal tissues, although the latter is seen less commonly than in ALCL-ALK+. Most cases of ALCL-ALK− demonstrate effacement of lymph node architecture by sheets of cohesive neoplastic cells with typical “hallmark” features. In contrast to the ALCL-ALK+, the small cell morphologic variant is not recognised.

Unlike its ALK+ counterpart, ALCL-ALK− shows a greater preservation of surface T-cell marker expression, while the expression of cytotoxic markers and epithelial membrane antigen (EMA) is less likely. Gene expression signatures and recurrent chromosomal imbalances are different in ALCL-ALK− and ALCL-ALK+, confirming that they are distinct entities at a molecular and genetic level.

ALCL-ALK− is clinically distinct from both ALCL-ALK+ and PTCL-NOS, with significant differences in prognosis among these three different entities. The 5-year overall survival of ALCL-ALK− is reported as 49% which is not as good as that of ALCL-ALK+ (at 70%), but at the same time it is significantly better than that of PTCL-NOS (32%).

### Enteropathy-Associated T-Cell Lymphoma (EATL)

EATL is an aggressive neoplasm which thought to be derived from the intraepithelial T-cells of the intestine. Two morphologically, immunohistochemically and genetically distinct types of EATL are recognised in the 2008 WHO classification: Type I (representing the majority of EATL) and Type II (comprising 10-20% of cases).

Type I EATL is usually associated with overt or clinically silent gluten-sensitive enteropathy, and is more often seen in patients of Northern European extraction due to high prevalence of celiac disease in this population.

Most commonly, the lesions of EATL are found in the jejunum or ileum (90% of cases), with rare presentations in duodenum, colon, stomach, or areas outside of the gastrointestinal tract. The intestinal lesions are usually multifocal with mucosal ulceration. Clinical course of EATL is aggressive with most patients dying of disease or complications of disease within 1 year.

The cytological spectrum of EATL type I is broad, and some cases may contain anaplastic cells. There is a polymorphous inflammatory background, which may obscure the neoplastic component in some cases. The intestinal mucosa in regions adjacent to the tumour often shows features of celiac disease with blunting of the villi and increased numbers of intraepithelial lymphocytes (IEL), which may represent lesional precursor cells.

By immunohistochemistry, the neoplastic cells are often CD3+CD4−CD8−CD7+CD5−CD56−βF1+, and contain cytotoxic granule-associated proteins (TIA-1, granzyme B, perforin). CD30 is partially expressed in almost all cases. CD103, which is a mucosal homing receptor, can be expressed in EATL.

Type II EATL, also referred to as monomorphic CD56+ intestinal T-cell lymphoma, is defined as an intestinal tumour composed of small- to medium-sized monomorphic T-cells that express both CD8 and CD56. There is often a lateral spread of tumour within the mucosa, and absence of an inflammatory background. The majority of cases express the γδ TCR, however there are cases associated with the αβ TCR.

Type II EATL has a more world-wide distribution than Type I EATL and is often seen in Asians or Hispanic populations, in whom celiac disease is rare. In individuals of European descent EATL, 11 represents about 20% of intestinal T-cell lymphomas, with a history of celiac disease in at least a subset of cases. The clinical course is aggressive.

### Hepatosplenic T-Cell Lymphoma (HSTL)

HSTL is an aggressive systemic neoplasm generally derived from γδ cytotoxic T-cells of the innate immune system, however, it may also be derived from αβ T-cells in rare cases. It is one of the rarest T-cell lymphomas, and typically affects adolescents and young adults (median age, 35 years) with a strong male predominance.

### Extranodal NK/T-Cell Lymphoma Nasal Type

Extranodal NK/T-cell lymphoma, nasal type, is an aggressive disease, often with destructive midline lesions and necrosis. Most cases are of NK-cell derivation, but some cases are derived from cytotoxic T-cells. It is universally associated with Epstein-Barr Virus (EBV).

### Cutaneous T-Cell Lymphoma

The method of the present disclosure may also be used to treat cutaneous T-cell lymphoma.

Cutaneous T-cell lymphoma (CTCL) is characterised by migration of malignant T-cells to the skin, which causes various lesions to appear. These lesions change shape as the disease progresses, typically beginning as what appears to be a rash and eventually forming plaques and tumours before metastasizing to other parts of the body.

Cutaneous T-cell lymphomas include those mentioned in the following illustrative, non-exhaustive list; mycosis fungoides, pagetoid reticulosis, Sézary syndrome, granulomatous slack skin, lymphomatoid papulosis, pityriasis lichenoides chronica, CD30+ cutaneous T-cell lymphoma, secondary cutaneous CD30+ large cell lymphoma, non-mycosis fungoides CD30− cutaneous large T-cell lymphoma, pleomorphic T-cell lymphoma, Lennert lymphoma, subcutaneous T-cell lymphoma and angiocentric lymphoma.

The signs and symptoms of CTCL vary depending on the specific disease, of which the two most common types are mycosis fungoides and Sezary syndrome. Classic mycosis fungoides is divided into three stages:
Patch (atrophic or nonatrophic): Nonspecific dermatitis, patches on lower trunk and buttocks; minimal/absent pruritus;
Plaque: Intensely pruritic plaques, lymphadenopathy; and
Tumor: Prone to ulceration

Sézary syndrome is defined by erythroderma and leukemia. Signs and symptoms include edematous skin, lymphadenopathy, palmar and/or plantar hyperkeratosis, alopecia, nail dystrophy, ectropion and hepatosplenomegaly.

Of all primary cutaneous lymphomas, 65% are of the T-cell type. The most common immunophenotype is CD4 positive. There is no common pathophysiology for these diseases, as the term cutaneous T-cell lymphoma encompasses a wide variety of disorders.

The primary etiologic mechanisms for the development of cutaneous T-cell lymphoma (ie, mycosis fungoides) have not been elucidated. Mycosis fungoides may be preceded by a T-cell-mediated chronic inflammatory skin disease, which may occasionally progress to a fatal lymphoma.

### Primary Cutaneous ALCL (C-ALCL)

C-ALCL is often indistinguishable from ALC-ALK− by morphology. It is defined as a cutaneous tumour of large cells with anaplastic, pleomorphic or immunoblastic morphology with more than 75% of cells expressing CD30. Together with lymphomatoid papulosis (LyP), C-ALCL belongs to the spectrum of primary cutaneous CD30-positive T-cell lymphoproliferative disorders, which as a group comprise the second most common group of cutaneous T-cell lymphoproliferations after mycosis fungoides.

The immunohistochemical staining profile is quite similar to ALCL-ALK−, with a greater proportion of cases staining positive for cytotoxic markers. At least 75% of the tumour cells should be positive for CD030. CD15 may also be expressed, and when lymph node involvement occurs, the differential with classical Hodgkin lymphoma can be difficult. Rare cases of ALCL-ALK+ may present with localized cutaneous lesions, and may resemble C-ALCL

### T-Cell Acute Lymphoblastic Leukaemia

T-cell acute lymphoblastic leukaemia (T-ALL) accounts for about 15% and 25% of ALL in paediatric and adult cohorts respectively. Patients usually have high white blood cell counts and may present with organomegaly, particularly mediastinal enlargement and CNS involvement.

The method of the present disclosure may be used to treat T-ALL which is associated with a malignant T cell which expresses a TCR comprising TRBC1.

### T-Cell Prolymphocytic Leukaemia

T-cell-prolymphocytic leukemia (T-PLL) is a mature T-cell leukaemia with aggressive behaviour and predilection for blood, bone marrow, lymph nodes, liver, spleen, and skin involvement. T-PLL primarily affects adults over the age of 30. Other names include T-cell chronic lymphocytic leukaemia, “knobby” type of T-cell leukaemia, and T-prolymphocytic leukaemia/T-cell lymphocytic leukaemia.

In the peripheral blood, T-PLL consists of medium-sized lymphocytes with single nucleoli and basophilic cytoplasm with occasional blebs or projections. The nuclei are usually round to oval in shape, with occasional patients having cells with a more irregular nuclear outline that is similar to the cerebriform nuclear shape seen in Sézary syndrome. A small cell variant comprises 20% of all T-PLL cases, and the Sézary cell-like (cerebriform) variant is seen in 5% of cases.

T-PLL has the immunophenotype of a mature (post-thymic) T-lymphocyte, and the neoplastic cells are typically positive for pan-T antigens CD2, CD3, and CD7 and negative for TdT and CD1a. The immunophenotype CD4+/CD8− is present in 60% of cases, the CD4+/CD8+ immunophenotype is present in 25%, and the CD4−/CD8+ immunophenotype is present in 15% of cases.

The TRBC1-positive T-cell malignancy may be selected from peripheral T-cell lymphoma (PTCL), peripheral T-cell lymphoma not otherwise specified (PTCL-NOS), angio-immunoblastic T-cell lymphoma (AITL), anaplastic large cell lymphoma (ALCL), enteropathy-associated T-cell lymphoma (EATL), hepatosplenic T-cell lymphoma (HSTL), extranodal NK/T-cell lymphoma nasal type, cutaneous T-cell lymphoma, primary cutaneous ALCL, or T cell prolymphocytic leukaemia and T-cell acute lymphoblastic leukaemia.

The patient may be of any age.

The age of the patient may be eighteen years or older.

The patient may have:
a) relapsed or refractory T-cell malignancy following at least one line of therapy, and
b) confirmed TRBC1-positive tumor.

Refractory T-cell malignancy is defined as a malignancy reappearing following at least one line of therapy.

Refractory T-cell malignancy is defined as not achieving a response or a complete response (CR) following at least one line of therapy.

TRBC1-tumour positivity refers to T cell malignancies where malignant cells express TRBC1. Methods to determine whether a T cell malignancy is TRBC1-positive are known by the skilled person and include polymerase chain reaction (PCR), sequencing, next-generation sequencing (NGS), Western blotting, flow cytometry, fluorescent microscopy, and immunohistochemistry (IHC). An exemplary NGS-based method of determining the TRBC gene type of a cell involving determining the J gene type expressed in said cell and inferring the TRBC gene type is described in Example 6 and WO2020025928.

In the method for treating a TRBC1-positive malignancy of the present disclosure, the patient may be administered a single dose of about 25×10⁶, 75×10⁶, 225×10⁶, 450×10⁶, or 900×10⁶ anti-TRBC1 CAR T cells.

The patient may be administered a single dose of 450×10⁶ anti-TRBC1 CAR T cells.

The administration of the anti-TRBC1 CAR T cells may be an intravenous injection. The intravenous injection may be through a Hickman line or peripherally inserted central catheter.

The patient may receive a pre-conditioning chemotherapy prior to administration of the anti-TRBC1 CAR T cells.

The pre-conditioning chemotherapy may be a lymphodepleting pre-conditioning treatment with fludarabine and cyclophosphamide. Patients may receive fludarabine and cyclophosphamide according to the dosing described below:
Day −6: FLU 30 mg/m² followed by CY 500 mg/m²
Day −5: FLU 30 mg/m² followed by CY 500 mg/m²
Day −4: FLU 30 mg/m²
Day −3: FLU 30 mg/m²

The pre-conditioning chemotherapy may be completed a minimum of 3 days (−1 day) prior to anti-TRBC1 CAR T cells infusion.

Patient survival may be longer than about 3 months, 6 months, 9 months, 12 months, 15 months, 18 months, 21 months, 24 months, 30 months, 36 months, 42 months, 48 months, or longer following administration of the anti-TRBC1 CAR T cells.

### Pharmaceutical Compositions

The method of the present disclosure may comprise the step of administering the agent in the form of a pharmaceutical composition.

The agent may be administered with a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as (or in addition to) the carrier, excipient or diluent, any suitable binder(s), lubricant(s), suspending agent(s), coating agent(s), solubilising agent(s), and other carrier agents.

### Administration

The administration of the agent can be accomplished using any of a variety of routes that make the active ingredient bioavailable. For example, the agent can be administered by oral and parenteral routes, intraperitoneally, intravenously, subcutaneously, transcutaneously, intramuscularly, via local delivery for example by catheter or stent.

Typically, a physician will determine the actual dosage which will be most suitable for an individual subject and it will vary with the age, weight and response of the particular patient. The dosage is such that it is sufficient to reduce or deplete the number of clonal T-cells expressing TRBC1.

### Other Terminology and Disclosure

As used herein and in the appended claims, the singular forms “a,” “and,” and “the” include plural referents unless the context clearly dictates otherwise. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

When a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of the present disclosure. All publications mentioned herein are incorporated herein by reference to disclose and describe the methods and/or materials for the purpose for which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible. This disclosure is intended to provide support for all such combinations.

As used herein, “may,” “may comprise,” “may be,” “can,” “can comprise” and “can be” all indicate something envisaged by the inventors that is functional and available as part of the subject matter provided.

Various abbreviations used herein follow.
AE Adverse event
AESI Adverse event of special interest
ALK Anaplastic lymphoma kinase
ALL Acute lymphoblastic leukaemia
ALT Alanine aminotransferase
ANC Absolute Neutrophil Count
ASBMT American Society for Blood and Marrow Transplantation
AST Aspartate aminotransferase
ASTCT American Society for Transplantation and Cellular Therapy
ATIMP Advanced therapy investigational medicinal product
aTRBC Anti-T cell receptor beta constant
aTRBC-CAR Anti-T cell receptor beta constant 1 chimeric antigen receptor
BCMA B cell maturation antigen
B-NHL B cell non-Hodgkin lymphoma
CAR Chimeric antigen receptor
CD3, 19, 20, 28, 68, 134 Cluster of differentiation 3, 19, 20, 28, 68 134
CHIM Cell handling instruction manual
CHOP Cyclophosphamide, Hydroxydoxorubicin, Oncovin, Prednisone
CMR Complete Metabolic Response
CMV Cytomegalovirus
CNS Central nervous system
CPK Creatine phosphokinase
CR Complete response
Cru Complete response unconfirmed
CRS Cytokine release syndrome
CT Computed tomography
CTCAE Common Terminology Criteria for Adverse Events
CTLA Cytotoxic T lymphocyte-associated protein
CY Cyclophosphamide
DFS Disease-free survival
DHAP Dexamethasone, High-dose Ara-C, Platinum
DIC Disseminated Intravascular Coagulation
DLBCL Diffuse Large B Cell Lymphoma
DLT Dose limiting toxicity
DMSO DDNA Deoxyribonucleic acid
DOR Duration of response
EBV Epstein Barr Virus
ECG Electrocardiogram
ECHO Echocardiogram
ECOG Eastern Cooperative Oncology Group
eCRF Electronic Case Report Form
EDC Electronic data capture
EDTA Ethylenediaminetetraacetic acid
EFS Event free survival
ESHAP Etoposide, Methylprednisolone, Ara-C, Platinum
EU European Union
EudraCT European Clinical Trials Database
FDA Food and Drug Administration
FDG Fluorodeoxyglucose
FFPE Formalin fixed paraffin embedded
FIH First-in-human
FLU Fludarabine
FLU-CY Fludarabine and cyclophosphamide
GCP Good Clinical Practice
G-CSF Granulocyte-Colony Stimulating Factor
GI Gastrointestinal
GM CSF Granulocyte-Macrophage Colony-Stimulating Factors
GMP Good Manufacturing Practice
HDAC Histone deacetylase
HIV Human immunodeficiency virus
HHV6 Human herpesvirus 6
HLH Haemophagocytic lymphohistiocytosis
HSCT Hematopoietic stem cell transplantation
HTLV Human T cell lymphotropic virus
IB Investigator's Brochure
ICANS Immune Effector Cell-associated Neurotoxicity Syndrome
ICE Ifosfamide, Carboplatin, Etoposide
ICF Informed Consent Form
ICH International Conference on Harmonisation
IDMC Independent Data Monitoring Committeeimethyl sulphoxide
IEC Independent Ethics Committee
IFN Interferon
IHC Immunohistochemistry
IL Interleukin
IMPD Investigational Medicinal Product Dossier
irAE Immune-related adverse event
IRB Institutional Review Board
i.v. Intravenous(ly)
JCV John Cunningham Virus
JOVI-1 JOVI-1 assay
LDH Lactate dehydrogenase
Ldi Longest transverse diameter of a lesion
LVEF Left ventricular ejection fraction
MAD Maximum administered dose
MAS Macrophage activation syndrome
MLV Murine Leukaemia Virus
MRI Magnetic resonance imaging
MTD Maximum tolerated dose
MUGA Multiple gated acquisition
NCI National Cancer Institute
NHL Non-Hodgkin lymphoma
NGS Next-Generation Sequencing
NOS Not otherwise specified
NSAID Non-steroidal anti-inflammatory drugs
OR Overall response
ORR Overall response rate
OS Overall survival
OX40 Also known as tumour necrosis factor receptor superfamily 4 [TNFRSF4] and cluster of differentiation 134 [CD134])
PBMCs Peripheral blood mononuclear cells
PCR Polymerase chain reaction
PD1 Programmed cell death protein 1
PD-L1 Programmed death-ligand 1
PET Positron emission tomography
PFS Progression-free survival
PI Prescribing information
PIS Patient Information Sheet
PPD Perpendicular diameter (cross product of the LDi and perpendicular diameter)
PR Partial response
PTCL Peripheral T cell lymphoma
PTT Partial thromboplastin time
q.a.d Quaque altera die (every other day)
QP Qualified Person
RCR Replication competent retrovirus
RP2D Recommended Phase II dose
RQR8 Ritux-QBEND/10-Ritux-CD8 sort-suicide gene generated as a marker/suicide gene for T cells
RSI Reference Safety Information
SAE Serious adverse event
scFv Single chain variable fragment
SCT Stem cell transplant/transplantation
SEC Safety Evaluation Committee
SmPC Summary of Product Characteristics
SPD Sum of the product of the perpendicular diameters for multiple lesions
SUSAR Suspected unexpected serious adverse reaction
TBD To be determined
TCR T cell Receptor
TLS Tumour lysis syndrome
TNF Tumour necrosis factor
TRBC T cell receptor beta constant
T-NHL T cell non-Hodgkin Lymphoma
ULN Upper limit of normal

### EXAMPLES

While the following examples describe specific embodiments, variations and modifications will occur to those skilled in the art. Accordingly, only such limitations as appear in the claims should be placed on the invention.

As described in the examples below, the advanced therapy medicinal product (ATMP) is an autologous, cell and gene therapy product that is manufactured by genetic modification of the patient's own T cells by ex vivo transduction with a MLV-derived retroviral vector that has been engineered to stably deliver the RQR8 and aTRBC1-CAR genes. The active substance is the genetically modified (RQR8/aTRBC1-CAR positive) T cells. This therapeutic cell product is also termed AUTO4 or huJovi1 CAR T cells. AUTO4 also contains non-transduced autologous lymphocytes. AUTO4 is presented for i.v. infusion in one or more CryoMACS® bag(s) as a cell dispersion in phosphate-buffered saline/EDTA/human albumin solution and a final concentration of 7.5% DMSO.

### Example 1—Heavy and Light Chain Anti-TRBC1 Binding Domain Selection

Humanised VH domains were constructed with the JOVI-1 VH CDRs following human VH frameworks: H-AF062256, H-EF177999, H-KF688165. Humanised VL domains were constructed with the 3aaz human framework.

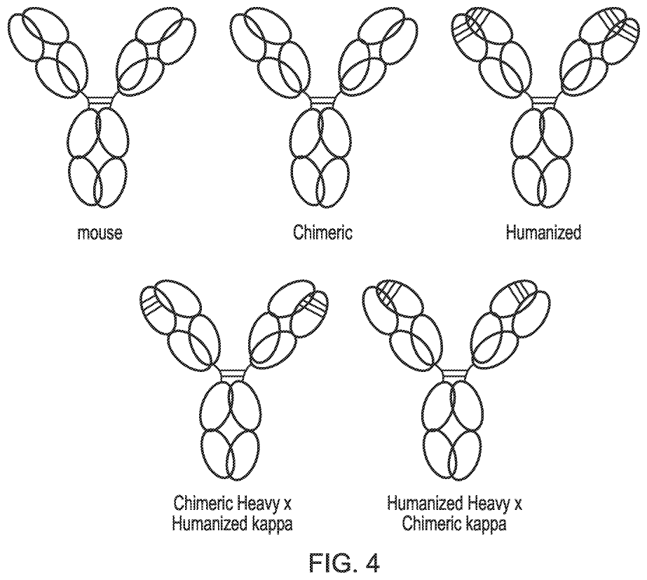

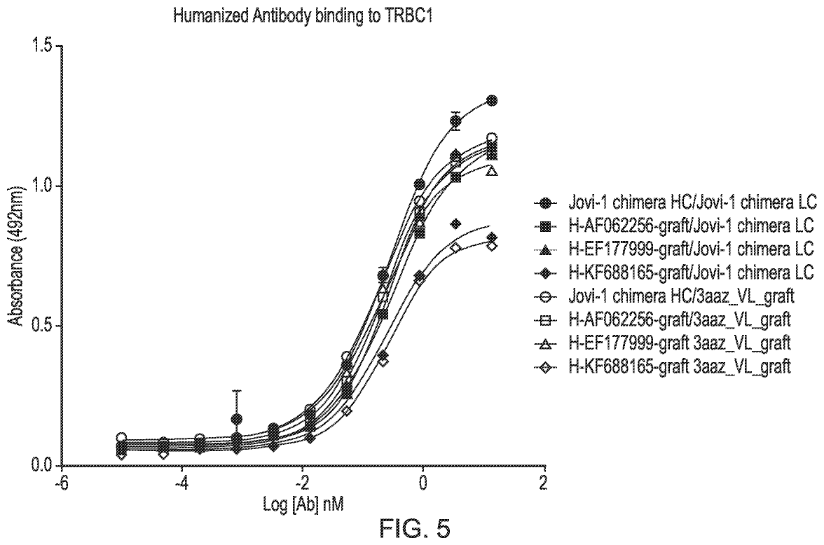

Antibodies were created with either a humanised VH domain and the murine JOVI-1 VL domain; the murine VH domain and a humanised VL domain; or a humanised VH domain and a humanised VL domain (see FIG. 4).

Binding to TRBC1 was tested by ELISA and the results are shown in FIG. 5.

All of the chimeric and humanised binder combinations were found to be capable of binding TRBC1 and binding was similar to the chimeric antibody having murine VH and VL domains.

### Example 2—Making and Testing Back Mutated Constructs

A series of back-mutated binders for human framework H-AF062256 were created, as shown in Table 1, in which back mutations are shown in bold.

TABLE 1

| | Heavy | Heavy chain |
|---|---|---|
| Construct | Chain | Mutations |
| Jovi Hum 8 | H-AF 1 | Q3, V20, M48, R71, T73 |
| Jovi Hum 16 | H-AF 2 | Q3, V20, M48, R71, K73 |
| Jovi Hum 17 | H-AF 3 | Q3, V20, M48, S71, T73 |
| Jovi Hum 18 | H-AF 4 | Q3, V20, M48, S71, K73 |
| Jovi Hum 19 | H-AF 5 | Q3, V20, I48, R71, T73 |
| Jovi Hum 20 | H-AF 6 | Q3, V20, I48, R71, K73 |
| Jovi Hum 21 | H-AF 7 | Q3, V20, I48, S71, T73 |
| Jovi Hum 22 | H-AF 8 | Q3, V20, I48, S71, K73 |
| Jovi Hum 23 | H-AF 9 | Q3, M20, M48, R71, T73 |
| Jovi Hum 24 | H-AF 10 | Q3, M20, M48, R71, K73 |
| Jovi Hum 25 | H-AF 11 | Q3, M20, M48, S71, T73 |
| Jovi Hum 26 | H-AF 12 | Q3, M20, M48, S71, K73 |
| Jovi Hum 27 | H-AF 13 | Q3, M20, I48, R71, T73 |
| Jovi Hum 28 | H-AF 14 | Q3, M20, I48, R71, K73 |
| Jovi Hum 29 | H-AF 15 | Q3, M20, I48, S71, T73 |
| Jovi Hum 30 | H-AF 16 | Q3, M20, I48, S71, K73 |
| Jovi Hum 31 | H-AF 17 | R3, V20, M48, R71, T73 |
| Jovi Hum 32 | H-AF 18 | R3, V20, M48, R71, K73 |
| Jovi Hum 33 | H-AF 19 | R3, V20, M48, S71, T73 |
| Jovi Hum 34 | H-AF 20 | R3, V20, M48, S71, K73 |
| Jovi Hum 35 | H-AF 21 | R3, V20, I48, R71, T73 |
| Jovi Hum 36 | H-AF 22 | R3, V20, I48, R71, K73 |
| Jovi Hum 37 | H-AF 23 | R3, V20, I48, S71, T73 |
| Jovi Hum 38 | H-AF 24 | R3, V20, I48, S71, K73 |
| Jovi Hum 39 | H-AF 25 | R3, M20, M48, R71, T73 |
| Jovi Hum 40 | H-AF 26 | R3, M20, M48, R71, K73 |
| Jovi Hum 41 | H-AF 27 | R3, M20, M48, S71, T73 |
| Jovi Hum 42 | H-AF 28 | R3, M20, M48, S71, K73 |
| Jovi Hum 43 | H-AF 29 | R3, M20, I48, R71, T73 |
| Jovi Hum 44 | H-AF 30 | R3, M20, I48, R71, K73 |
| Jovi Hum 45 | H-AF 31 | R3, M20, I48, S71, T73 |
| Jovi Hum 46 | H-AF 32 | R3, M20, I48, S71, K73 |

The binders had a humanised VL domain comprising the 3aaz framework.

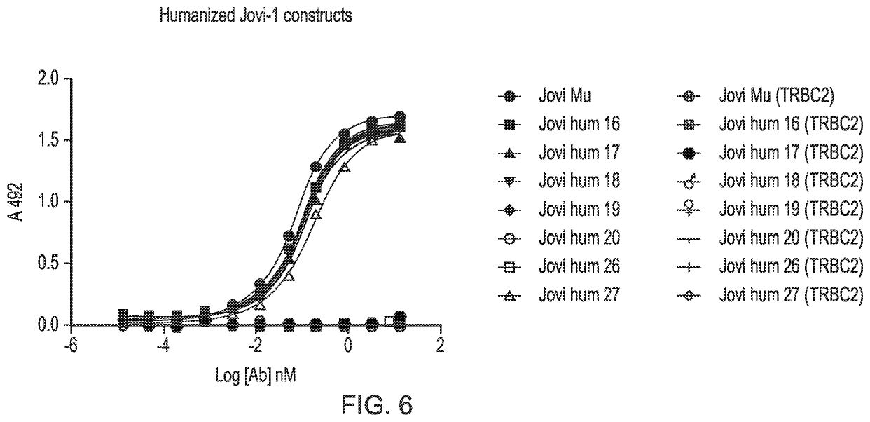

Binding to TRBC1 and TRBC2 was tested by ELISA and the results are shown in FIG. 6. All the constructs bound TRBC1 but not TRBC2 and displayed a similar EC50 to the chimeric antibody having murine VH and VL domains (Jovi-Mu). This shows that following CDR grafting, the specificity and affinity of the murine antibody is retained in the humanised antibodies.

### Example 3—Generation of a Chimeric Antigen Receptor (CAR) with a Humanised Anti-TRBC1 Antigen Binding Domain

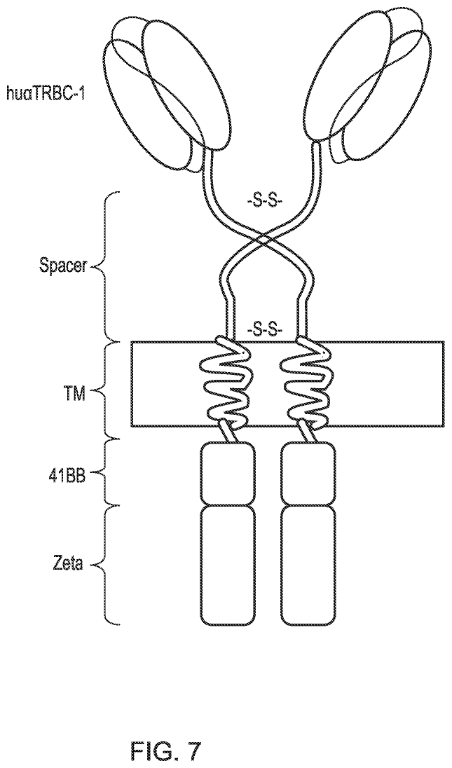

A second generation CAR was designed having a 41 BB and CD3 zeta endodomain and an antigen binding domain comprising a humanised JOVI-1 scFv (H-AF1, 3aaz) as illustrated schematically in FIG. 7. Primary human T-cells from normal donors were transduced with retroviral vectors expressing the anti-TRBC1 CAR.

### Example 4—Inclusion Criteria for Treatment

Patients met all the following criteria:
1 Male or female, aged ≥18 years.
2 Willing and able to give written, informed consent to be screened for TRBC1 positive T-NHL.
3 Confirmed diagnosis of selected T-NHL including:
   a Peripheral T cell lymphoma NOS, or
   b Angioimmunoblastic T cell lymphoma or
   c Anaplastic large cell lymphoma.
4 Confirmed TRBC1 positive tumour.
5 Relapsed or refractory disease and have had 1 prior lines of therapy.
6 FDG-avid measurable disease on PET/CT per Lugano classification.
7 Eastern Cooperative Oncology Group (ECOG) Performance Status 0 or 1.
8 Adequate Bone Marrow Function without the requirement for ongoing blood products and meets the following criteria:
   a Absolute neutrophil count ≥1.0×10⁹/L
   b Absolute lymphocyte count ≥0.5×10⁹/L (at entry and prior to leukapheresis)
   c Haemoglobin ≥80 g/L
   d Platelets ≥75×10⁹/L
9 Adequate renal, hepatic, pulmonary, and cardiac function defined as:
   a Creatinine clearance (as estimated by Cockcroft Gault) ≥60 cc/min.
   b Serum alanine aminotransferase/aspartate aminotransferase ≤2.5× upper limit of normal (ULN).
   c Total bilirubin ≤25 μmol/L (1.5 mg/dL), except in patients with Gilbert's syndrome.
   d Left ventricular ejection fraction (LVEF) ≥50% by echocardiogram (ECHO) or multiple gated acquisition (MUGA) cardiac scan, unless the institutional lower limit of normal is lower.
   e Baseline oxygen saturation ≥92% on room air and ≤Grade 1 dyspnoea
10 For females of childbearing potential (defined as <2 years after last menstruation or not surgically sterile), a negative serum or urine pregnancy test must be documented at screening, prior to pre-conditioning and confirmed before receiving the first dose of study treatment. For females who are not postmenopausal (<24 months of amenorrhea) or who are not surgically sterile (absence of ovaries and/or uterus), a highly effective method of contraception together with a barrier method must be used from the start of the pre-conditioning stage and for at least 12 months after the last dose of AUTO4 (study treatment). They must agree not to donate eggs (ova, oocytes) for the purposes of assisted reproduction during the study and for 12 months after receiving the last dose of study drug.
11 For males, it must be agreed that two acceptable methods of contraception are used from the start of the pre-conditioning stage and for at least 12 months after the last dose of AUTO4 (one by the patient—usually a barrier method, and one by the patient's partner—refer to Appendix 3). Also, that sperm will not be donated during the treatment period and for at least 12 months after the last dose of study treatment.
12 No contra-indications for leukapheresis or the pre-conditioning regimen.

### Example 5—Exclusion Criteria

Patients excluded include:
1 Patients with T cell leukaemia.
2 Females who are pregnant or lactating.
3 Prior treatment with investigational gene therapy or approved gene therapy or genetically engineered cell therapy product or allogeneic stem cell transplant.
4 Known history or presence of clinically relevant central nervous system (CNS) pathology such as epilepsy, paresis, aphasia, stroke within prior 3 months, severe brain injuries, dementia, Parkinson's disease, cerebellar disease, organic brain syndrome, uncontrolled mental illness, or psychosis. Patients with a known history or prior diagnosis of optic neuritis or other immunologic or inflammatory disease affecting the CNS.
5 Current or history of CNS involvement by malignancy.
6 Clinically significant, uncontrolled heart disease (New York Heart Association Class Ill or IV heart failure, uncontrolled angina, severe uncontrolled ventricular arrhythmias, sick-sinus syndrome, or electrocardiographic evidence of acute ischaemia or Grade 3 conduction system abnormalities unless the patient has a pacemaker) or a recent (within 12 months) cardiac event.
   a Uncontrolled cardiac arrhythmia (patients with rate-controlled atrial fibrillation are not excluded).
   b Evidence of pericardial effusion
7 Patients with evidence of uncontrolled hypertension or with a history of hypertension crisis or hypertensive encephalopathy.
8 Patients with a history (within 3 months) or evidence of deep vein thrombosis or pulmonary embolism requiring ongoing therapeutic anticoagulation at the time of pre-conditioning.
9 Patients with active gastrointestinal (GI) bleeding.
10 Patients with any major surgical intervention in the last 3 months.
11 Active infectious bacterial, viral or fungal disease (hepatitis B virus, hepatitis C virus, human immunodeficiency virus [HIV], human T cell lymphotropic virus [HTLV] or syphilis) requiring treatment.
12 Active autoimmune disease requiring immunosuppression.
13 History of other neoplasms unless disease free for at least 2 years (adequately treated carcinoma in situ, curatively treated non-melanoma skin cancer, breast or prostate cancer on hormonal therapy are allowed).
14 Prior treatment with programmed cell death protein 1 (PD1), programmed death-ligand 1 (PD-L1), or cytotoxic T lymphocyte-associated protein 4 targeted therapy (CTLA-4), or tumour necrosis factor (TNF) receptor superfamily agonists including CD134 (OX40), CD27, CD137 (41BB), and CD357 (glucocorticoid-induced TNF receptor family-related protein) within 6 weeks prior to AUTO4 infusion.
15 The following medications are excluded:
   a Steroids: Therapeutic doses of prednisone/equivalent of more than 20 mg per day are prohibited within 7 days prior to leukapheresis or pre-conditioning chemotherapy administration. However, physiological replacement, topical, and inhaled steroids are permitted.
   b Cytotoxic chemotherapies within 2 weeks prior to leukapheresis or AUTO4 infusion.
   c Antibody therapy use within 2 weeks prior to AUTO4 infusion, or five half-lives of the respective antibody, whichever is longer
   d Live vaccine within 4 weeks prior to enrolment.
16 Research participants receiving any other investigational agents, or concurrent biological, chemotherapy, or radiation therapy.
17 Use of rituximab (or rituximab biosimilar) within the last 6 months prior to AUTO4 infusion.
18 Patients, who in the opinion of the Investigator, may not be able to understand or comply with the safety monitoring requirements of the study.

### Example 6—NGS Assay Used to Select TRBC1 Positive Patients

TRBC1 expression on FFPE tumour biopsy tissue was evaluated using the LymphoTrack Dx TRB Assay-MiSeq to identify clonal T cell receptor beta (TRB) gene rearrangements using Next-Generation Sequencing (NGS) with the Illumina MiSeq. There is a cluster of TRBV genes that are shared by both types of TCR-13 chain. Located downstream of the TRBV, there are two TRBD-TRBJ-TRBC clusters which are TRBD1-TRBJ1-TRBC1 followed by TRBD2-TRBJ2-TRBC2 with a 2.6 kb intergenic region in between. The VDJ recombination occurs at the genomic level, while at the transcription level, the rearranged VDJ is joined with TRBC1 or TRBC2 via splicing. NGS analysis of healthy human T cells demonstrates, that in most cases, TRBJ1 links to C1 (99.9%) and TRBJ2 links to C2 (99.99%). This formed the rationale for genotyping the TCR-I3-related VDJ in tumour containing tissue samples via NGS and using the information of the clonal VDJ rearrangement to indicate if the tumour expresses TRBC1 or TRBC2.

In brief, a minimum of six and a maximum of fifteen 5 μm sections were cut from the archival or newly acquired FFPE biopsy tissue and placed into Eppendorf tubes. Genomic DNA was extracted and purified from the tissue. The extracted and purified genomic DNA was then placed into a single multiplex master mix for a PCR reaction. The PCR amplicons were then be purified to remove excess primers, nucleotides, salts and enzymes using the Agencourt AMPure XP system. The purified amplicons were quantified using the KAPA Library Quantification Kit for Illumina Platforms. Finally, 600 μL of the final prepared library were loaded onto a MiSeq Reagent Cartridge and the MiSeq run was started. The sequencing data was analysed using the LymphoTrack Dx Software-MiSeq package. The merged Read Summary Report was used to identify the top merged read sequences and their frequencies.

A patient was identified as having a TRBC1 positive T-NHL if the tumour sample was determined to be clonal by the LymphoTrack Dx TRB Assay—MiSeq; where the presence of a J1 sequence determines C1 positivity.

### Example 7—Leukapheresis for AUTO4 Manufacturing

Patients underwent an unstimulated leukapheresis for the generation of AUTO4. Leukapheresis was performed within 30 days of infectious disease testing and was done following the standard institutional processes. An additional sample was taken for a second infectious screen on the day of leukapheresis (or within 7 days after).

In general, leukapheresis was performed at least 35 days before the planned AUTO4 dosing date, as AUTO4 manufacture and release can take approximately 1 month. Based on emerging data this window can be changed. If the product is ready early the patient may receive the product early.

The leukapheresate is the starting material for the manufacture of the ATIMP, AUTO4. The total cell number that is required for successful manufacture varies according to the dose level. Typically, a double volume leukapheresis was performed. The target collection is between 1×10⁹-5×10⁹ PBMCs. The leukapheresate was transported for generation of AUTO4 at a temperature of 2 to 8° C. as soon as possible and within 48 hours, ideally within 24 hours.

Bridging chemotherapy was prescribed to the patient during the AUTO4 manufacturing period at the discretion of the physician and in accordance with the exclusion criteria and washout periods outlined in the eligibility criteria. Each leukapheresate was identified by a unique patient identification number plus any additional patient identifiers as allowed per local regulations (typically initials and date of birth).

### Example 8—AUTO4 Manufacturing

AUTO4 is an autologous, cell and gene therapy investigational medicinal product that is manufactured by genetic modification of the patient's own T cells by ex vivo transduction with a MLV-derived retroviral vector that has been engineered to stably deliver the RQR8 and aTRBC1-CAR genes. The active substance is the genetically modified (RQR8/aTRBC1-CAR positive) T cells. AUTO4 also contains non-transduced autologous lymphocytes.

The starting material for generation of AUTO4 is unstimulated leukapheresate taken from the patient. This may require insertion of central venous access and is a day case procedure to collect peripheral blood mononuclear cells (PBMCs) only.

The total cell number that is required for successful manufacture varies according to the dose level. Typically, a double volume leukapheresis was performed. The target collection was between 1×10⁹-5×10⁹ PBMCs. The leukapheresate was transported for generation of AUTO4 at a temperature of 2 to 8° C. as soon as possible and within 48 hours, ideally within 24 hours.

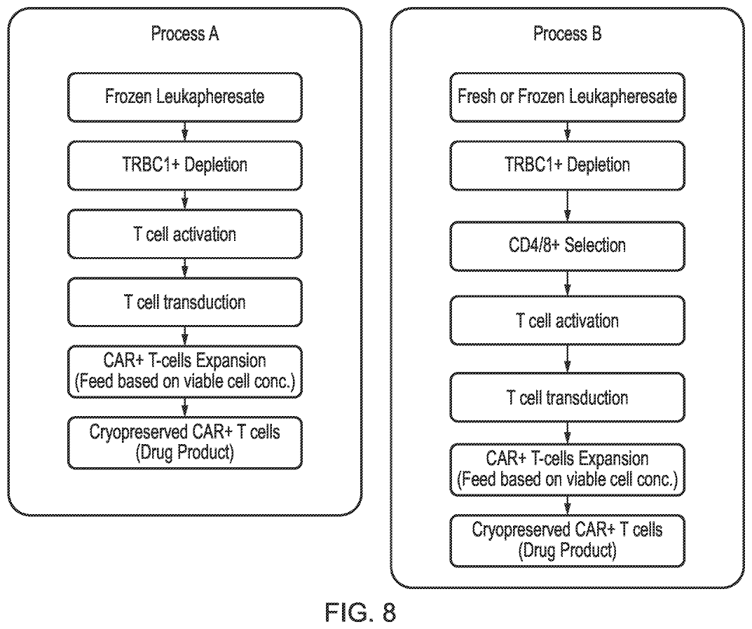

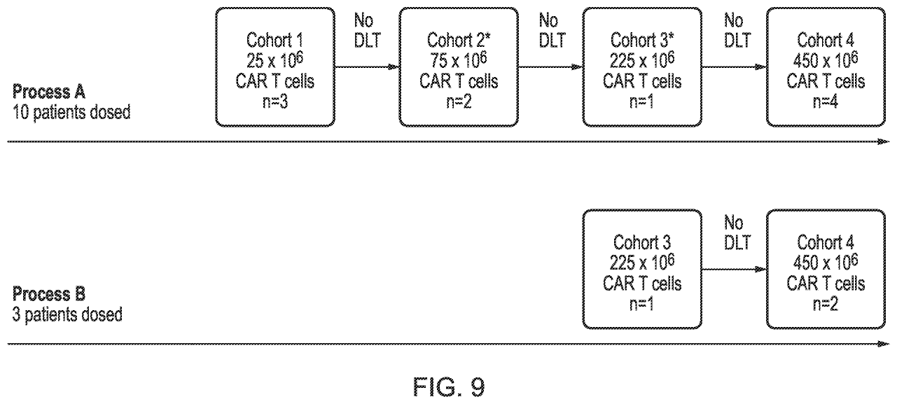

AUTO4 is manufactured by genetic modification of the patient's T cells by ex vivo transduction with a MLV-derived retroviral vector by Process A or Process B as set out in FIG. 8. Briefly, T cells were obtained from the leukapheresate taken from each lymphoma patient who has been identified as TRBC1 positive during study screening. After a TRBC1 positive cell depletion of the leukapheresate, the T cells within the TRBC1 negative cell collected fraction were activated and then transduced with the retroviral vector. Cells were then expanded (drug substance) and then cryopreserved (drug product). Drug product from initial AUTO4 process was used to dose four patient cohorts (Process A, FIG. 9).

The process was further optimised (Process B, FIG. 9) to enable a greater number of less differentiated CAR positive T-cells (naïve/central memory phenotype with greater proliferative potential) to be achieved at an earlier harvest timepoint compared to the current process. Briefly, Process B used fresh apheresis, optimised seeding and transduction, and performed CD4+/CD8+ selection. The optimisation resulted in reduced manufacturing time up to 4 days compared to Process A. Drug product manufactured using Process B was used to dose two patient cohorts.

AUTO4 was cryopreserved in one or more CryoMACS® bag(s) and stored in a vapour-phase liquid nitrogen environment prior to administration.

CryoMACS® freezing bags are single use, sterile containers intended for a single cycle of freezing, storage (down to −196° C.), and subsequent thawing (at 37° C.) of AUTO4 cells. The CryoMACS© freezing bags are comprised of a freezing bag (with access ports) as the primary containment for AUTO4 and an overwrap bag as secondary containment. Additionally, the CryoMACS® freezing bag has a built-in label pocket, which allows for the insertion of the patient label to include patient identification and product specifications for AUTO4. As part of the bag assembly, two spike ports are available which allow access to the bag contents for therapeutic use of the product (via attachment of a sterile transfusion assembly).

AUTO4 was presented for i.v. infusion in one or more of the CryoMACS® bag(s) as a cell dispersion in phosphate-buffered saline/EDTA/human albumin solution and a final concentration of 7.5% DMSO.

### Example 9—Pre-Conditioning Chemotherapy

Patients that met eligibility requirements and for whom the AUTO4 product was released to specification, received a lymphodepleting pre-conditioning treatment with Fludarabine (FLU) and Cyclophosphamide (CY) before AUTO4 infusion. The pre-conditioning phase began with administration of pre-conditioning chemotherapy and ended with the beginning of treatment with AUTO4 infusion. During this phase, AEs associated with pre-conditioning chemotherapy as well as use of concomitant medications were collected.

Prior to administration of pre-conditioning chemotherapy, patients underwent clinical and laboratory assessments as per the Schedule of Assessments (appearing at the end of the Examples herein) and the physician determined if the patient was fit to receive pre-conditioning chemotherapy. If considered to be fit, patients received a lymphodepleting pre-conditioning treatment with FLU and CY for 4 days (starting Day −6 [−1 day]) and timed to end 3 days (−1 day) before AUTO4 infusion.

### Pre-Conditioning Chemotherapy Dose and Regimen

Patients received fludarabine (FLU) and cyclophosphamide (CY) according to the dosing described below; FLU was given first.
Day −6: FLU 30 mg/m² followed by CY 500 mg/m²
Day −5: FLU 30 mg/m² followed by CY 500 mg/m²
Day −4: FLU 30 mg/m²
Day −3: FLU 30 mg/m²

The pre-conditioning chemotherapy was completed a minimum of 3 days (−1 day) prior to AUTO4 infusion.

Fludarabine was given by i.v. infusion over 30 minutes in sodium chloride 0.9%. For patients with renal impairment (glomerular filtration rate 30 to 60 mL/min/1.73 m² [corrected]), the dose of FLU was reduced per routine clinical practice (generally by 25%).

Cyclophosphamide was given by i.v. infusion over 30 minutes. Adequate pre- and post-hydration for up to 4 to 6 hours (or as per institutional practice) was given post-infusion to induce diuresis. Use of mesna for the prescribed dose was generally considered unnecessary but may be considered based on institutional practice. Cyclophosphamide dose was reduced if the leukocyte count is <2500 cells/μL with 6 hours post-hydration.

Anti-emetic prophylaxis was given as per standard institutional policy. Prophylaxis for TLS with allopurinol and i.v. fluids was given if clinically necessary.

### Example 10—Dosing

It is contemplated herein that by comparing the cellular distribution and frequency of B cells and T cells it is possible to estimate the relative potential for on-target off-tumour toxicities due to cytolysis of the normal cell population. The distribution of T cells and B cells within human organs is generally similar. The major distribution of both T and B cells is within lymphoid organs (lymph nodes, spleen, tonsils, thymus, bone marrow and lymphoid-associated tissue). T and B cells are also abundant in the blood, as well as part of the cellular infiltrate of multiple organs such as heart, lung, liver, various glandular tissue, and reproductive tissues. Tissue cross reactivity studies have noted T cell, but not B cell, distribution in the cerebral cortex of the brain, blood vessels, kidney, pituitary gland, placenta, skeletal muscle, skin, testes and thyroid. Additionally, neither B nor T cells were found in the cerebellum of the brain, spinal cord, eye and nerve.

A comparison of the relative numbers of T and B cells from normal tissues has been made (Sathaliyawala et al. 2013, Immunity 38(1):187-197). The frequency of B and T cells from blood and eight different healthy tissues including multiple lymphoid tissues (spleen, inguinal, mesenteric and bronchial/lung-draining lymph nodes) and mucosal tissues including the lung, small intestine regions (jejunum, ileum) and colon tissues (blood, spleen, inguinal lymph node) was compared. The frequency of T cells outnumbered B cells in all sites except the spleen—by 2-4-fold in blood and lymph nodes, 5-8 fold in ileum and colon and >15-20 fold in the lung and jejunum. A similar number of B and T cells were found in the spleen.

In summary, whilst there is a slightly higher cellular distribution of T cells across major organs, the frequency of T cell distribution within major organs and blood are significantly higher compared to B cells. Whilst targeting TRBC1 cells will only ablate approximately one third of normal T cells, there is likely to be more cytolysis of normal T cells within the lung and jejunum compared to historical experience in targeting B cells with CD19 CARs.

However, a significant number of these cells would have been depleted by the preconditioning chemotherapy. An additional factor is the target antigen density on normal cells. The antigen density of TCR on normal T cells is about 100,000 copies per T cell (Schodin et al. 1996, Immunity 5(2):137-146) which is approximately 5 times higher than the antigen density of CD19 on normal B cells. Generally the CAR T cell response against target cells, including the production of cytokines such as interleukin (IL)-2, Interferon (IFN)-γ and Granzyme-B, follows a sigmoid curve and higher antigen density is likely to generate a saturated response (Nguyen et al. 2016, J. Clin. Oncol. 34(15_suppl):10536-10536).

Considering the current experience with CD19 CARs in ALL, DLBCL and CLL involving patients with significant amount of antigen positive disease in various organs, it is contemplated herein that it is less likely that the acute toxicity will be significantly worse than that experienced with CD19 CARs and likely manageable with current safety management guidelines.

### Starting Dose Rationale

A starting dose of 25×10⁶ RQR8/aTRBC1-CAR positive T cells (single dose) was proposed, which corresponds to a dose of approximately 0.33×10⁶ RQR8/aTRBC1-CAR positive T cells/kg based on an average person's weight of 75 kg. The primary consideration for the starting dose for the FIH study is patient safety. CAR T cell dosing is a complex area and traditional dose determination methods based on pre-clinical data are not transposable for cell and gene therapies. CAR T cells expand in vivo and the CAR T cell expansion (or effective dose) differs from patient to patient. Consequently, toxicity cannot be accurately correlated to the administered starting dose with CAR T cell therapies. Therefore, the choice of the starting dose has primarily been based on CAR-related clinical trial literature, especially data from the two major CAR classes, anti-CD19 CAR targeting ALL and B cell non-Hodgkin lymphoma (B-NHL)/diffuse large B cell lymphoma and anti-B cell maturation antigen (BCMA) CARs targeting multiple myeloma.

The proposed starting dose of 25×10⁶ RQR8/aTRBC1-CAR positive T cells is at the lower end of starting doses in anti-CD19 CAR T clinical trials. Similar starting doses were used by other CAR studies targeting novel antigen anti-BCMA CAR (Ali et al. 2016, Blood 128(13):1688-1700). The proposed starting dose of 25×10⁶ RQR8/aTRBC1-CAR positive T cells (AUTO4) for T cell lymphomas is approximately one-sixth the dose of 2×10⁶ CD19(CD28z)-CAR T cells/kg, determined to be the MTD of autologous CD19 CAR T cells in National Institutes of Health studies. It is also half the starting dose of 0.66×10⁶ CAR T cells/kg (approximately 50×10⁶ CAR T cells in total) with HuCAR-19, a humanised CD19 (CD28-ζ) CAR (Brudno et al. 2016, Blood 127(26):3321-3330). The other factors considered in determining the starting dose were the comparative prevalence of normal T cell population (and TRBC1 subpopulation) in relation to B cells and the density of antigen expression on T versus B cells.

The current RQR8/aTRBC1-CAR construct used for the generation of AUTO4 includes a 41BB-ζ endodomain for the CAR and this configuration is unlikely to have significantly greater propensity for proliferation or toxicity compared to the CD19(CD28z)-CARs currently in clinical development. Even considering that the normal peripheral TRBC1 positive T cell number is likely to be 2 to 3 times higher than the normal peripheral B cell numbers (Morbach et al. 2010, Clin Exp Immunol 162(2):271-279), though a significant percent of the T cells will be depleted by the pre-conditioning chemotherapy. A starting dose that is 3 to 6-fold lower than active dose of CD19 CAR T cells is likely to be safe. Another component potentially associated with toxicity (especially in BALL) is disease burden. Considering the similarities between B and T cell lymphoma, it is contemplated herein that the acute toxicity profile is likely to be similar to CD19 (4-1BBz)-CARs in B cell lymphomas where a correlation to disease burden has not been demonstrated. Therefore, considering the rarity of patients with these tumours, the poor overall prognosis and given the safety considerations described above, the proposed starting dose of 25×10⁶ CAR T cell (approximately 0.33×10⁶ CAR T cells/kg) was considered an appropriate starting dose.

A starting dose of 225×10⁶ RQR8/aTRBC1-CAR positive T cells (single dose) for Process B was proposed, which corresponds to a dose of approximately 2.97×10⁶ RQR8/aTRBC1-CAR positive T cells/kg based on an average person's weight of 75 kg. This choice was based on the preliminary safety results of tested lower doses and the product phenotype of the two manufacturing processes indicating a less differentiated CAR T-cell product with Process B. Process B was optimized to enable a greater number of less differentiated CAR positive T cells to be achieved at an earlier harvest timepoint compared to the current process.

### Rationale for Maximum Administered Dose (MAD)

A maximum administered dose (MAD) of 900×10⁶ RQR8/aTRBC1-CAR T cells was chosen based on the experience with CD19 CARs in B cell lymphoma. This MAD choice also took into consideration the favourable safety profile and high level of clinical activity of AUTO3 in DLBCL (Ramakrishnan et al 2020, Abstract 600, ASH 2020). Additionally, higher doses of CAR T-cells therapy have been documented in the NHL treatment setting (Hay and Turtle 2017, Drugs. 77(3): 237-245). For example, in the University of Pennsylvania's CTL019 Phase 2 study, a CD19 (41BB-ζ) CAR dose was administered with a median dose of 3.1×10⁸ (range 0.1-6.0×10⁸). Considering that the design of the AUTO4 CAR is similar to the University of Pennsylvania's CAR, with a 41BB-ζ co-stimulatory domain, a MAD of 900×10⁶, was considered a safe and appropriate target maximal dose.

### Rationale for Re-Treatment Dosing

Chimeric antigen receptor T cell therapies are generally administered once, undergo significant expansion in vivo upon contact with the target antigen expressed on tumour cells and, particularly where a 41 BB-ζ co-stimulatory domain is incorporated into the CAR, persist longterm in a proportion of patients (Maude et al. 2014, N Engl J Med 371(16):1507-1517). It is contemplated herein that AUTO4 will have similar expansion and persistence in vivo to the CD19 CAR positive T cells with 41BB-ζ co-stimulatory domain, such as those utilised in the University of Pennsylvania studies, rendering the need for re-dosing unnecessary (Schuster et al. 2016, 58th Annual Meeting and Exposition. San Diego, CA, USA, American Society of Hematology). On rare occasion, a re-treatment dose may be given if the patient meets the criteria for re-treatment.

### Example 11—AUTO4 Administration

AUTO4 was administered as a single rapid infusion on Day 0 in an in-patient setting.

Premedication with diphenhydramine/chlorpheniramine and paracetamol/acetaminophen was given prior to infusion of AUTO4, but steroids was not given as part of premedication.

TABLE 2
(Process A) Dose Levels and Treatment Cohorts

| | | | Total RQR8/ | |
|---|---|---|---|---|
| | | | aTRBC1-CAR | |
| | | Pre-conditioning (Flu & CY; | Positive | |
| Dose | Treatment | Days −6, −5, −4, −3 | T Cells | Number of |
| Levels | Cohorts | [−1 day]) | (Day 0) | Patients |
| Dose Level | Cohort 1 | Yes | 25 × 10⁶ | 3 treated |
| 1 | | | | |
| Dose Level | Cohort 2 | Yes | 75 × 10⁶ | 1-6* |
| 2 | | | | |
| Dose Level | Cohort 3 | Yes | 225 × 10⁶ | 1-6* |
| 3 | | | | |
| Dose Level | Cohort 4 | Yes | 450 × 10⁶ | 3-6** |
| 4 | | | | |
| Dose Level | Cohort 5 | Yes | 900 × 10⁶ | 3-6** |
| 5 | | | | |

CAR = chimeric antigen receptor; FLU-CY = fludarabine and cyclophosphamide; RQR8/aTRBC1 = Ritux QBEnd/10-Ritux-cluster of differentiation 8 sort-suicide gene/anti-T cell receptor beta 1.
*For Cohorts 2 (75 × 10⁶ cells) and 3 (225 × 10⁶ cells) if there is no CAR-T expansion in any of the patients treated (with at least one patient treated at that dose) together with no Grade ≥ 1 CRS/Neurotoxicity or ≥Grade 2 AUTO4 -related adverse events in the first 28 days after AUTO4 infusion, SEC may approve escalation to next level. If any CAR-T expansion (above the assay limit of detection) is seen the cohort must have a minimum of 3 patients treated to be considered complete (per rolling six study design)
**From Cohort 4 (450 × 10⁶ cells) onwards, standard rolling six design will apply with a minimum of 3 patients treated per cohort. Cohort is expanded from 3 to 6 patients when 1 patient has a DLT.

TABLE 3
(Process B) Dose Levels and Treatment Cohorts

| | | | Total RQR8/ | |
|---|---|---|---|---|
| | | Pre-conditioning | aTRBC1-CAR | |
| | | (Flu & CY; Days | Positive | |
| Dose | Treatment | −6, −5, −4, | T Cells | Number of |
| Levels | Cohorts | −3 [−1 day]) | (Day 0) | Patients |
| Dose Level | Cohort 3b | Yes | 225 × 10⁶ | 1-6* |
| 3 | | | | |
| Dose Level | Cohort 4b | Yes | 450 × 10⁶ | 3-6 ** |
| 4 | | | | |
| Dose Level | Cohort 5b | Yes | 900 × 10⁶ | 3-6 ** |
| 5 | | | | |

*For Cohort 3b (225 × 10⁶ cells) if there is no CAR-T expansion in any of the patients treated (with at least one patient treated at that dose) together with no Grade ≥ 1 CRS/Neurotoxicity or ≥Grade 2 AUTO4 -related adverse events in the first 28 days after AUTO4 infusion, the SEC may approve escalation to the next level. If any CAR-T expansion (above the assay limit of detection) is seen, the cohort must have a minimum of 3 patients treated to be considered complete (per rolling six study design)
** For Cohort 4b (450 × 10⁶ cells), the standard rolling six design will apply with a minimum of 3 patients treated per cohort. The cohort is expanded from 3 to 6 patients when 1 patient has a DLT.

AUTO4 was infused as follows. In brief:
AUTO4 was thawed rapidly in a 37° C. water bath under sterile conditions.
The entire contents of the bag(s) was given as an i.v. infusion using a syringe or gravity aided infusion through a central or large bore peripheral venous access over a few minutes (maximum 30 minutes from AUTO4 being thawed to preserve cell viability).
A leukodepleting filter was not used for the infusion of the T cell product.
The infusion line and the bag(s) were flushed to ensure all cells have been administered.
If there were multiple bags of cell product, one bag was thawed and safely infused before the second one was thawed.
The time between completion of thawing and completion of infusion did not exceed 30 minutes.

FIG. 9 shows the number of patients dosed for each of six different cohorts, three cohorts received product prepared by Process A shown in FIG. 8 and two cohorts received product prepared by Process B shown in FIG. 8.

Table 4 below shows the baseline characteristics of treated patients.

TABLE 4

| Baseline Characteristics | Total (N = 13) |
|---|---|
| Age, median (range) | 57 (34-72) |
| ECOG 0/1, n (%) | 4 (31%), 9 (69%) |
| Median prior lines of treatment (range) | 2 (1-5) |
| Stage of Lymphoma at screening | |
| I/II | 2 (15%) |
| III/IV | 11 (85%) |
| Lymphoma Subtype, n (%) | |
| Peripheral T-cell lymphoma-NOS (PTCL-NOS) | 7 (54%) |
| Anaplastic large cell lymphoma (ALCL), | 2 (15%) |
| ALK-negative | |
| Angioimmunoblastic T-cell lymphoma (AITL) | 4 (31%) |
| Prior Autologous Stem Cell Transplant, n (%) | 3 (23%) |
| CD30+ Immunophenotype for T-Cell NHL, n (%) | 9 (69%)* |
| Bridging therapy YES, n (%) | 10 (77%) |

### Example 12—Re-Treatment of Patients

As noted above, it is contemplated that most patients will receive a single dose of AUTO4, as part of their treatment. However, some patients may qualify for a re-treatment upon treating physician request. This re-treatment may be for patients in whom there has been no CAR T-cell engraftment (e.g absence or low levels of CAR T-cell expansion) and could use either remaining CAR T-cells from the initial manufacturing process (if there is AUTO4 product leftover), or by a new AUTO4 manufacturing (e.g repeating the leukapheresis procedure and manufacturing process), if the patient clinical status allows (per treating physician decision). Specific criteria for re-treatment are described below and individual risk-benefit considerations should be taken into account upon treating physician discussion.

Prior to re-treatment, the patient must meet the following criteria:
1 Circulating levels of AUTO4 were low (<0.2×10⁹/dL AUTO4 cells) or undetectable and no significant anti-tumour effect (no CR) after the first dose, and the first treatment dose was considered to be sub therapeutic.
OR
2 There was objective clinical evidence of anti-tumour activity following the previous AUTO4 infusion (i.e. Stable Disease or better).
OR
3 The patient had evidence of progressive disease in the context of declining levels of AUTO4. Circulating levels AUTO4 cells must have been low (<0.2×10⁹/dL) or undetectable for at least 2 weeks prior to the second infusion.
AND both of the following:
1 The patient tolerated the first infusion without dose-limiting or other severe (≥Grade 4) or unmanageable toxicity for a follow-up period of at least 28 days.
2 The patient still fulfils the trial entry criteria required to tolerate another pre-conditioning treatment and AUTO4 infusion.

Patients undergoing a second AUTO4 infusion should receive the same pre-conditioning chemotherapy. The decision to re-treat a patient will be made by the treating physician.

### Example 13—Primary Objectives and Endpoints/Outcomes

Primary objectives and endpoints for treatment are presented in Table 3 and Table 4, respectively.

TABLE 3
Primary Objectives and Endpoints for Phase I

| Objectives | Endpoints |
|---|---|
| To assess the safety and | Incidence of Grade 3 to 5 toxicity |
| tolerability of AUTO4 | occurring within 60 days of AUTO4 |
| administration. | [symbol] |
| To identify the RP2D and MTD, | Frequency of DLT of AUTO4 within 28 |
| if an MTD exists, of AUTO4. | days of AUTO4 infusion. |

DLT = dose limiting toxicity;
MTD = maximum tolerated dose;
RP2D = recommended Phase II dose [symbol]
indicates data missing or illegible when filed

TABLE 4
Primary Objectives and Endpoints for Phase II

| Objectives | Endpoints |
|---|---|
| To assess the clinical activity of | Overall response (CR + PR) rate post |
| AUTO4 when administered at the | AUTO4 infusion |
| RP2D | |

CR = complete response;
PR = partial response

### Example 14—Secondary Objectives and Endpoints

Secondary objectives and endpoints are presented in Table 5.

TABLE 5
Secondary Objectives and Endpoints for Phase I and II

| Objectives | Endpoints |
|---|---|
| To assess the overall | Frequency and severity of all AEs and |
| safety and tolerability | SAEs |
| of AUTO4. | Incidence and severity of opportunistic |
| | infections following AUTO4 infusion. |
| To evaluate the feasibility | Proportion of patients (who are TRBC1 |
| of generating the ATIMP, | positive and undergo leukapheresis), |
| AUTO4. | for whom an AUTO4 product can be |
| | generated (feasibility). |
| To evaluate the overall | Determine the CR rate following |
| clinical efficacy of AUTO4. | treatment with AUTO4. |
| | Evaluate clinical outcomes including |
| | DOR, DFS, [symbol] |
| To determine the expansion | RQR8/aTRBC1-CAR positive T cells as |
| and persistence of AUTO4 | determined by PCR and/or flow |
| following infusion. | cytometry at a range of time points |
| Duration of TRBC1 positive | in the peripheral blood. |
| T cell aplasia. | Enumeration of circulating TRBC1 |
| | positive T cells assessed by flow |
| | cytometry at a range of time points |
| | in the peripheral blood. |

AE = adverse event; ATIMP = advanced therapy investigational medicinal product; CR = complete response; DFS = disease free survival; DOR = duration of response; PCR = polymerase chain reaction: OS = overall survival; PFS = progression free survival; PR = partial response; SAE = serious adverse event; TRBC = T cell receptor beta constant. [symbol]
indicates data missing or illegible when filed

### Example 15—Exploratory Objectives

The exploratory objectives for treatment are as follows:
To determine the time course and magnitude of cytokine release evaluated using an appropriate assay.
To assess the duration of depletion of circulating TRBC1 positive T cells as determined by flow cytometry on the peripheral blood and correlate this with disease response.
To assess antibody and or T cell mediated immune responses against AUTO4.
To characterize the relationship between the CAR T cell phenotype/genomics and persistence.
To investigate if there is a relationship between parameters of activity, percent TRBC1 positive T cells, level of TRBC1 expression on tumour cells and CAR T cell phenotype. To investigate if there is a relationship between incidence and severity of CRS, neurotoxicity or other toxicity, and tumour burden, percent TRBC1 positive T cells, level of TRBC1 expression on tumour cells pre-treatment and CAR T cell phenotype.

### Example 16—Pharmacokinetics, Pharmacodynamics and Biomarker Evaluation

Blood-based pharmacodynamics biomarkers were evaluated in all patients as described in the Schedule of Assessments. Peripheral blood biomarkers were assessed pre- and post-AUTO4 treatment. Assessment at additional or fewer time points was performed based on emerging data.

### Evaluation of AUTO4 Persistence in Peripheral Blood

Two validated assays were used to measure the expansion/persistence of RQR8/aTRBC1-CAR positive T cells at the time points indicated in the Schedule of Assessments. Flow cytometry was used to measure the frequency of RQR8/aTRBC1-CAR positive T cells per microliter of whole blood and/or a PCR assay was used to quantify the number of copies of the RQR8/aTRBC1-CAR transgene per microgram of genomic DNA and/or per cell in peripheral blood.

AUTO4 CARs were detected in the blood in only two of ten patients. One patient had peak expansion at day 13 and persistence to day 120 (latest day tested). One other patient had AUTO4 CARs detected ten minutes and 1 hour post infusion.

### Evaluation of AUTO4 Persistence and TRBC1 Expression in Lymph Node Tumour

When possible lymph node tumour biopsy samples were assessed for the presence of CAR T-cells and TRBC1 expression in the tumour. The assessments were performed using ddPCR or immunohistochemistry. Biopsy samples were taken at the timepoints outlined in the Schedule of Assessments (Footnote 9).

Table 6 below shows the results from ddPCR analysis of AUTO4 CARs in patient lymph node biopsies.

TABLE 6

| | | Lymph node |
|---|---|---|
| | Sample collection | ddPCR* |
| Patient ID | post infusion | (VCN/ug DNA) |
| 01 (Cohort 1 25 × 10⁶ cells) | Day 75 | 19,695 |
| 09 (Cohort 1 25 × 10⁶ cells) | Day 11 | 520 |
| 022 (Cohort 1 25 × 10⁶ cells) | Day 7 | 4,141 |
| 059 (Cohort 4 450 × 10⁶ cells) | Day 8 | 2,525 |
| 055 (Cohort 4 450 × 10⁶ cells) | Day 13 | 10,302 |

*Limit of detection: 11 copies/ug DNA; Limit of quantification: 20 copies/ug DNA.

Five out of five evaluable lymph node FFPE samples showed the presence of AUTO4 CARs by ddPCR.

FIG. 11 shows images from immunohistochemistry of a lymph node biopsy. AUTO4 CAR T-cells were detected at day 75 post-infusion in a lymph node biopsy of a patient who achieved complete remission. Approximately 4.4% of all CD3+ T-cells in the biopsy were CAR T-cells.

The absence of CARs in blood and the presence in lymph nodes suggests fast homing of CARs to tumour sites.

For patients treated with AUTO4 product manufactured with Process B, Table 9 below shows the results from ddPCR analysis of AUTO4 CARs in patient lymph node biopsies.

TABLE 9

| | Sample collection | Lymph node ddPCR* |
|---|---|---|
| Patient ID | post infusion | (VCN/ug DNA) |
| 082 (225 × 10⁶ cells) | Day 9 | 808 |
| | Day 28 | 5,353 |
| 103 (450 × 10⁶ cells) | Day 5 | 171,700 |
| | Month 2 | 308 |
| | Month 5 | 505 |
| 115 (450 × 10⁶ cells) | Day 14 | 808 |
| 117 (450 × 10⁶ cells) | Day 7 | 111 |

### Evaluation of RCR in Peripheral Blood

As per health authorities' guidelines, tests were performed to evaluate and monitor the presence of RCR by PCR in whole blood or PBMCs.

### TRBC1 Positive T Cell Aplasia

AUTO4 targets polyclonal TRBC1+ T-cells. If AUTO4 expansion is observed, TRBC1 positive T-cell aplasia may occur. Blood samples were collected for the analysis of the levels of TRBC1 positive and TRBC1 negative T cell subsets (including those that are CD4+ and CD8+) in accordance with the Schedule of Assessments. Samples were analyzed centrally with a validated Flow Cytometry assay.

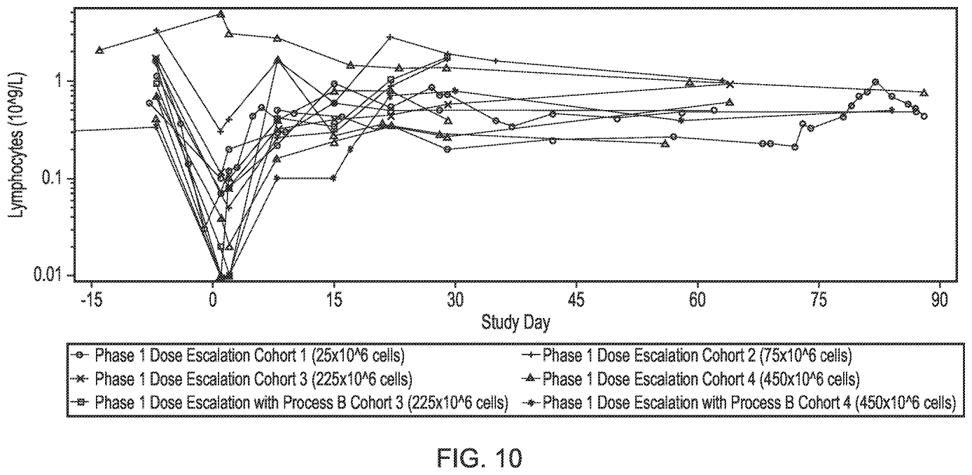

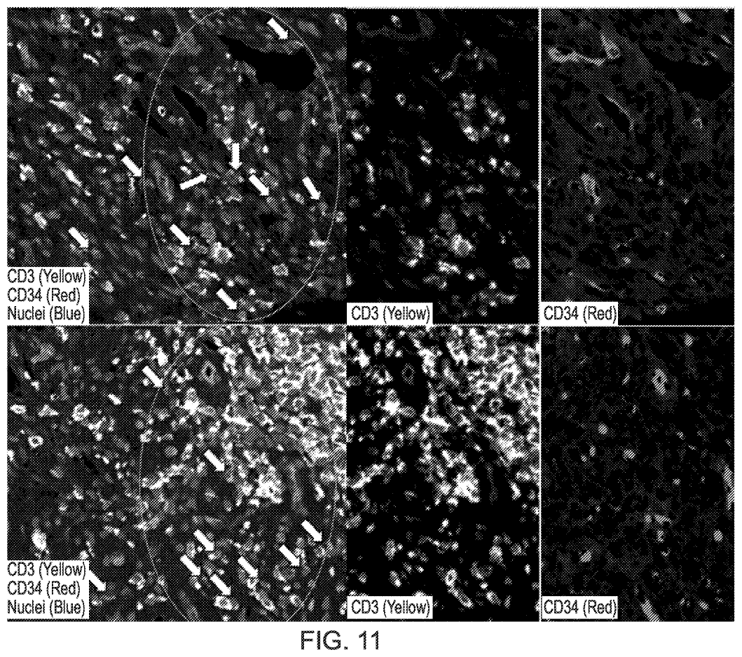

Results for Process A Cohorts 1-4 are shown in FIG. 10. Transient lymphopenia was observed after Flu/Cy and AUTO4 infusion.

### Insertional Mutagenesis

Blood samples were stored but not analysed (unless clinical evidence dictated) as per the Schedule of Assessments for insertional mutagenesis unless there was evidence that AUTO4 is no longer present. The result will allow identification of any potential relationship between AUTO4 treatment and the development of any new malignancy.

### Exploratory Biomarker Assessments

Serum cytokine profile: The serum cytokine profile (using a minimum dataset of TNF-α, interferon-γ, and IL-6) was measured using a highly sensitive, reproducible, and validated cytokine assay at time points indicated in the Schedule of Assessments. Additional samples were taken where clinically indicated, for example during CRS. Blood samples for cytokine measurements were frozen and batched for analysis or assayed using fresh serum. Serum was also used for measurement of other biomarkers as appropriate.

Immunological/genomic phenotyping: PBMCs were isolated from whole blood following standard procedures and cryopreserved in liquid nitrogen for later immunological assessment or assessed immediately. PBMCs were used for various immunological assessments such as phenotyping by flow cytometry, genomic analysis and other assays as developed. Immunophenotyping of PBMCs was evaluated at selected time points (per the Schedule of Assessments) and dependent upon a minimum frequency of RQR8/aTRBC1-CAR positive cells.

TRBC1 expression on lymphoma tissue: If sufficient FFPE tumour tissue was provided at baseline, the expression of TRBC1 on lymphoma cells was evaluated by IHC.

PD-L1 expression on lymphoma cells: If sufficient FFPE tumour tissue was provided at baseline, the expression of PD-L1 on lymphoma cells was evaluated by IHC, and where possible from tumour tissue samples collected between Day 7 and 21 and/or at progression of disease.

### Immunogenicity Analysis

Detection of human anti-CAR T cell responses and antibodies, or related antibodies, was measured in cryopreserved PBMCs and serum. Serum or plasma samples at selected time points, for example at Day 0, end of DLT evaluation period and Month 3 to 6, were analysed if clinically indicated. Additional samples were analysed if clinically indicated e.g., if AUTO4 cells become undetectable or at relapse.

### Example 17—Efficacy Evaluation

Response evaluations were conducted as specified in the Schedule of Assessments and included the following: CT and PET using [¹⁸F]-fluorodeoxyglucose (FDG), physical examination, and other procedures as necessary. MRI was used to evaluate sites of disease that could be adequately imaged using CT (in cases where MRI was desirable, the MRI was obtained at baseline and at all subsequent response evaluations).

Radiographic Image Assessments (CT/MRI) Disease response was assessed using CT scans with i.v. (and oral as necessary) contrast of the neck, chest, abdomen, pelvis and any other location where disease was present at Screening, and whole body [¹⁸F]-FDG-PET scans.

Radiological assessments were performed as outlined in the Schedule of Assessments.

### Positron Emission Tomography Scan

Positron emission tomography using [¹⁸F]-FDG is important for the complete assessment of response and progression. Whole body [¹³F]-FDG-PET scan (skull base to the proximal femur) was required at Screening and then performed as outlined in the Schedule of Assessments.

For patients who achieved a complete metabolic response (CMR), disease assessments after Month 6 were based on CT scans alone, if clinically appropriate. Assessment of PET results was based on published criteria. Visual assessment was considered adequate for determining whether a PET scan was positive and use of the standardized uptake value was not necessary. A positive scan was defined as focal or diffuse [¹⁸F]-FDG uptake above background in a location incompatible with normal anatomy or physiology, without a specific standardized uptake value cut-off. Other causes of false-positive scans were ruled out. Exceptions include mild and diffusely increased [¹⁸F]-FDG uptake at the site of moderate- or large-sized masses with an intensity that is lower than or equal to the mediastinal blood pool, hepatic or splenic nodules 1.5 cm with [¹⁸F]-FDG uptake lower than the surrounding liver/spleen uptake, and diffusely increased bone marrow uptake within weeks after treatment.

### Tissue Biopsy

If relapse occurs after CMR or disease progression is suspected (e.g., new or enlarging lesion(s) detected on PET/CT scan), tissue biopsy should be used as unscheduled assessment to confirm PD, if in an accessible location which would not put the patient at any safety risk per treating physician judgment. Newly acquired tumour tissue can be provided from either an excisional biopsy or core needle biopsy. Although an excisional biopsy is preferred, the physician may choose to provide a core needle biopsy if a lesion is suitable.

### Example 18—Efficacy Criteria

### Assessment of Disease Response and Progressive Disease

Efficacy assessments were performed according to the Lugano Classification (Cheson et al. 2014, J Clin Oncol 32(27):3059-3068) (Table 7 below).

TABLE 7

| Response | Site | PET-CT-Based Response | CT-Based Response |
|---|---|---|---|
| Complete | | Complete metabolic response | Complete radiologic response |
| | | | (all of the following) |
| | Lymph nodes | Score 1, 2, or 3^{a} with or without a | Target nodes/nodal masses must |
| | and | residual mass on 5PS^{b} | regress to ≤1.5 cm in the longest |
| | extralymphatic | It is recognised that in Waldeyer's | transverse diameter of the lesion |
| | sites | ring or extranodal sites with high | (LDi). |
| | | physiologic uptake or with | No extralymphatic sites of disease |
| | | activation within spleen or marrow | |
| | | (e.g., with chemotherapy or | |
| | | myeloid colony-stimulating | |
| | | factors), uptake may be greater | |
| | | than normal mediastinum and/or | |
| | | liver. In this circumstance, | |
| | | complete metabolic response may | |
| | | be inferred if uptake at sites of | |
| | | initial involvement is no greater | |
| | | than surrounding normal tissue | |
| | | even if the tissue has high | |
| | | physiologic uptake | |
| | Non-measured | Not applicable | Absent |
| | lesion | | |
| | Organ | Not applicable | Regress to normal |
| | enlargement | | |
| | New lesions | None | None |
| | Bone marrow | No evidence of FDG-avid disease | Normal by morphology; if |
| | | in marrow | indeterminate, IHC negative |
| Partial | | Partial metabolic response | Partial remission (all of the |
| | | | following) |
| | Lymph nodes | Score 4 or 5^{b} with reduced uptake | ≥50% decrease in sum of the product |
| | and extra- | compared with baseline and | of the perpendicular diameters for |
| | lymphatic | residual mass(es) of any size | multiple lesions (SPD) of up to 6 |
| | sites | At interim, these findings suggest | target measurable nodes and |
| | | responding disease | extranodal sites |
| | | At end of treatment, these findings | When a lesion is too small to |
| | | indicate residual disease | measure on CT, assign 5 mm × 5 mm |
| | | | as the default value |
| | | | When no longer visible, 0 × 0 mm |
| | | | For a node >5 mm × 5 mm, but |
| | | | smaller than normal, use actual |
| | | | measurement for calculation |
| | Non-measured | Not applicable | Absent/normal, regressed, but no |
| | lesion | | increase |
| | Organ | Not applicable | Spleen must have regressed by >50% |
| | enlargement | | in length beyond normal |
| | New lesions | None | None |
| | Bone Marrow | Residual uptake higher than uptake | Not applicable |
| | | in normal marrow but reduced | |
| | | compared with baseline (diffuse | |
| | | uptake compatible with reactive | |
| | | changes from chemotherapy | |
| | | allowed). If there are persistent | |
| | | focal changes in the marrow in the | |
| | | context of a nodal response, | |
| | | consideration should be given to | |
| | | further evaluation with MRI or | |
| | | biopsy or an interval scan | |
| No response | | No metabolic response | Stable disease |
| or stable | | | |
| disease | | | |
| | Lymph nodes | Score 4 or 5 with no significant | <50% decrease from baseline in SPD |
| | and | change in FDG uptake from | of up to 6 dominant, measurable |
| | extralymphatic | baseline at interim or end of | nodes and extranodal sites; no |
| | sites | treatment | criteria for progressive disease are |
| | | | met. |
| | Non-measured | Not applicable | No increase consistent with |
| | lesion | | progression |
| | Organ | Not applicable | No increase consistent with |
| | enlargement | | progression |
| | New lesions | None | None |
| | Bone marrow | No change from baseline | Not applicable |
| Progressive | | Progressive metabolic disease | Progressive disease requires at |
| disease | | | least 1 of the following: |
| | Individual | Score 4 or 5 with an increase in | Cross product of the LDi and |
| | target | intensity of uptake from baseline | perpendicular diameter (PPD) |
| | nodes/nodal | and/or | progression: |
| | masses | | |
| | Extranodal | New FDG-avid foci consistent with | An individual node/lesion must be |
| | lesions | lymphoma at interim or end-of- | abnormal with: |
| | | treatment assessment | LDi > 1.5 cm, and |
| | | | increase by ≥50% from PPD nadir, and |
| | | | an increase in LDi or SDi from nadir: |
| | | | 0.5 cm for lesions ≤ 2 cm |
| | | | 1.0 cm for lesions > 2 cm |
| | | | In the setting of splenomegaly, the |
| | | | splenic length must increase by >50% |
| | | | of the extent of its prior |
| | | | increase beyond baseline (e.g., a 15- |
| | | | cm spleen must increase to >16 cm). |
| | | | If no prior splenomegaly, must |
| | | | increase by at least 2 cm from |
| | | | baseline. |
| | | | New or recurrent splenomegaly |
| | Non-measured | None | New or clear progression of pre- |
| | lesions | | existing non-measured lesions |
| | New lesions | New FDG-avid foci consistent with | Regrowth of previously resolved |
| | | lymphoma rather than another | lesions |
| | | aetiology (e.g., infection, | A new node > 1.5 cm in any axis |
| | | inflammation). If uncertain | A new extranodal site > 1.0 cm in any |
| | | regarding aetiology of new lesions, | axis; if <1.0 cm in any axis, its |
| | | biopsy or interval scan may be | presence must be unequivocal and |
| | | considered | must be attributable to lymphoma |
| | | | Assessable disease of any size |
| | | | unequivocally attributable to |
| | | | lymphoma |
| | Bone marrow | New or recurrent FDG-avid foci | New or recurrent involvement |

5PS = 5-point scale; CT = computed tomography; FDG = fluorodeoxyglucose; IHC = immunohistochemistry; LDi = longest transverse diameter of a lesion; MRI = magnetic resonance imaging; PET = positron emission tomography; PPD = cross product of the LDi and perpendicular diameter; SDi = shortest axis perpendicular to the LDi; SPD = sum of the product of the perpendicular diameters for multiple lesions.
^{a}A score of 3 in many patients indicates a good prognosis with standard treatment, especially if at the time of an interim scan. However, in trials involving PET where de-escalation is investigated, it may be preferable to consider a score of 3 as inadequate response (to avoid undertreatment). Measured dominant lesions: Up to 6 of the largest dominant nodes, nodal masses, and extranodal lesions selected to be clearly measurable in 2 diameters. Nodes should preferably be from disparate regions of the body and should include, where applicable, mediastinal and retroperitoneal areas. Non-nodal lesions include those in solid organs (e.g., liver, spleen, kidneys, lungs), GI involvement, cutaneous lesions, or those noted on palpation. Non-measured lesions: Any disease not selected as measured, dominant disease and truly assessable disease should be considered not measured. These sites include any nodes, nodal masses, and extranodal sites not selected as dominant or measurable or that do not meet the requirements for measurability, but are still considered abnormal, as well as truly assessable disease, which is any site of suspected disease that would be difficult to follow quantitatively with measurement, including pleural effusions, ascites, bone lesions, leptomeningeal disease, abdominal masses, and other lesions that cannot be confirmed and followed by imaging. In Waldeyer's ring or in extranodal sites (e.g., GI tract, liver, bone marrow), fluorodeoxyglucose uptake may be greater than in the mediastinum with complete metabolic response, but should be no higher than surrounding normal physiologic uptake (e.g., with marrow activation as a result of chemotherapy or myeloid growth factors).
^{b}PET 5PS: 1, no uptake above background; 2, uptake ≤ mediastinum; 3, uptake > mediastinum but ≤ liver; 4, uptake moderately > liver; 5, uptake markedly higher than liver and/or new lesions; X, new areas of uptake unlikely to be related to lymphoma

For suspected baseline disease that may not be detected by PET, e.g., skin, eye, GI wall change, appropriate screening assessments, e.g., skin biopsy, eye imaging assessment, endoscopy, were required. In the efficacy follow up, these disease locations were re-examined, and a CR was only called if disease from all anatomical locations was resolved, evidenced by relevant assessments.

### Definition of Measurable and Assessable Disease

Eligible patients had PET-positive disease at baseline (FDG-avid disease corresponding with a 5-point scale score of 4 or 5). Patients who received bridging therapy after study enrollment had a PET/CT scan performed after completion of bridging therapy. Patients who did not have PET-positive disease (5-point scale score of 4 or 5) after bridging treatment were excluded from the primary efficacy analysis. Patients with PET-positive disease at baseline but without measurable disease per CT scan were included in the primary efficacy analysis.

For radiological assessments based on CT scan (or MRI), measurable sites of disease were defined as lymph nodes, lymph node masses, or extranodal sites of lymphoma. Each measurable site of disease had to be greater than 1.5 cm in the long axis regardless of short axis measurement, or greater than 1.0 cm in the short axis regardless of long axis measurement, and clearly measurable in two perpendicular dimensions. Measurement was determined by imaging evaluation. All other sites of disease were considered assessable, but not measurable. Up to six measurable sites of disease, clearly measurable in two perpendicular dimensions, were followed for each patient. Measurable sites of disease were chosen such that they were representative of the patient's disease (this includes splenic and extranodal disease). If there were lymph nodes or lymph node masses in the mediastinum or retroperitoneum larger than 1.5 cm in two perpendicular dimensions, at least one lymph node mass from each region was always be included. In addition, selection of measurable lesions was from as disparate regions of the body as possible.

All other sites of disease were considered assessable. Assessable disease included objective evidence of disease that was identified by radiological imaging, physical examination, or other procedures as necessary, but was not measurable as defined above. Examples of assessable disease included bone lesions; mucosal lesions in the GI tract; effusions; pleural, peritoneal, or bowel wall thickening; disease limited to bone marrow; and groups of lymph nodes that were not measurable but were thought to represent lymphoma. In addition, if more than six sites of disease were measurable, these other sites of measurable disease were included as assessable disease.

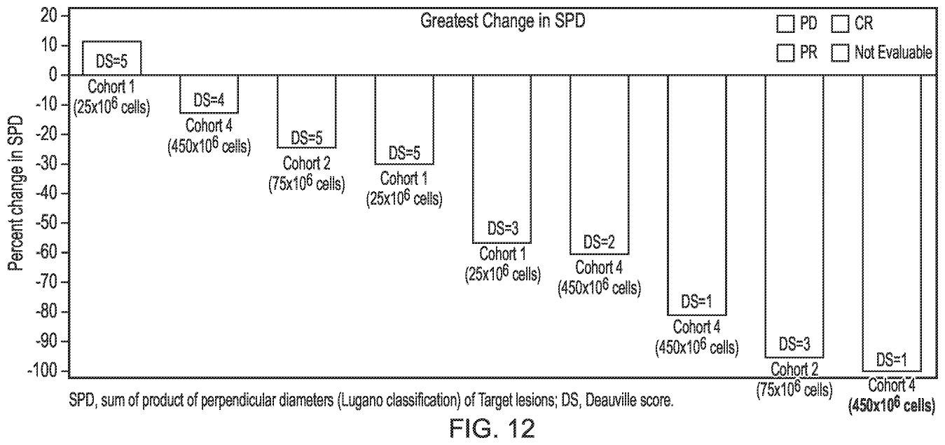

FIG. 12 summarizes the percent change in the sum of product of perpendicular diameters (SPD) (Lugano classification) of target lesions in four patients at the highest Process A AUTO4 dose. Three of four patients achieved complete metabolic response (CMR) at month 1.

### Efficacy Endpoints

The efficacy endpoints were Overall Response Rate (ORR), DOR, DFS, PFS, and OS.

Overall response rate: CR or PR by the Criteria for Response Assessment of NHL (i.e. Lugano Classification).

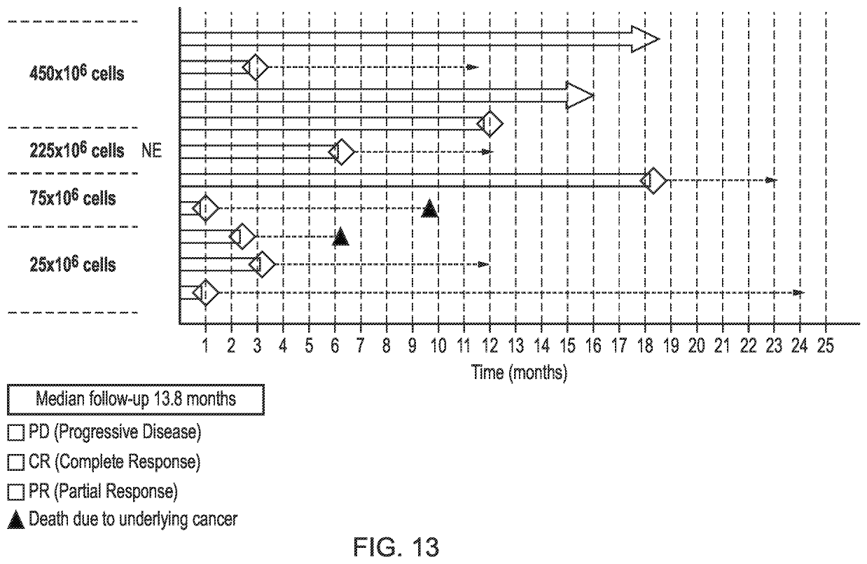

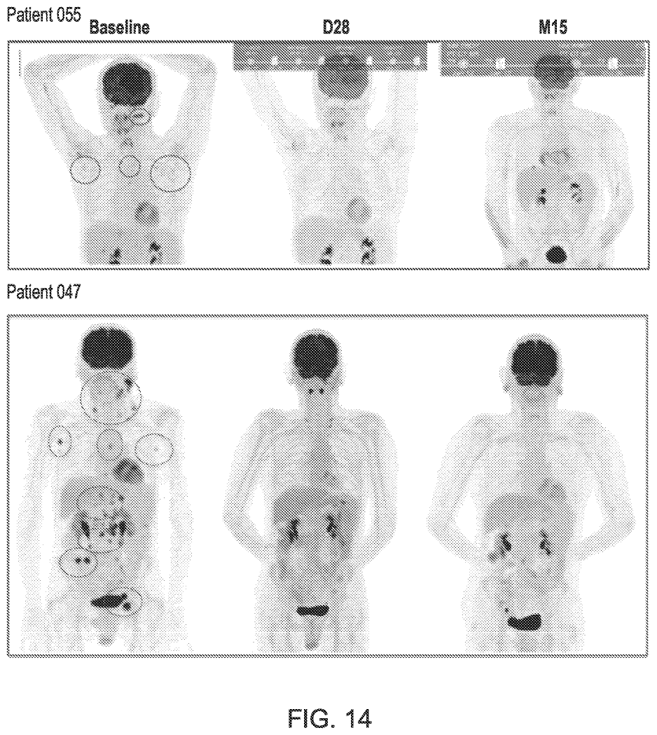

The proportion of patients achieving PR and CR at 1 and 3, and 6 months post-AUTO4 infusion was determined. FIG. 13 shows four of four patients at the 450×10⁶ cell dose achieved a response, and two of four patients at that dose remained in CMR beyond 12 months. FIG. 14 shows PET-CT images for the two patients.

The time to response (PR+CR) and the time to CR was calculated. These were defined as the time from the first treatment of AUTO4 to the response (either PR or CR as appropriate).

Duration of response: DOR is defined as the time from the first observed CR or PR to documented disease progression or death due to any cause, for patients who are considered as responders.

Progression-free survival: PFS is defined as the time from the first treatment of AUTO4 to documented disease progression/relapse or death due to any cause.

Overall survival: OS is defined as the time from the first treatment of AUTO4 to death due to any cause. Date of death was recorded.

Efficacy in patients treated with AUTO4 manufactured with Process B Three patients were dosed with AUTO4 (Process B):
1 patient at 225×10⁶ cell dose (PTCL-NOS), and
2 patients at 450×10⁶ cell dose (PTCL-NOS, ALK-neg. ALCL).

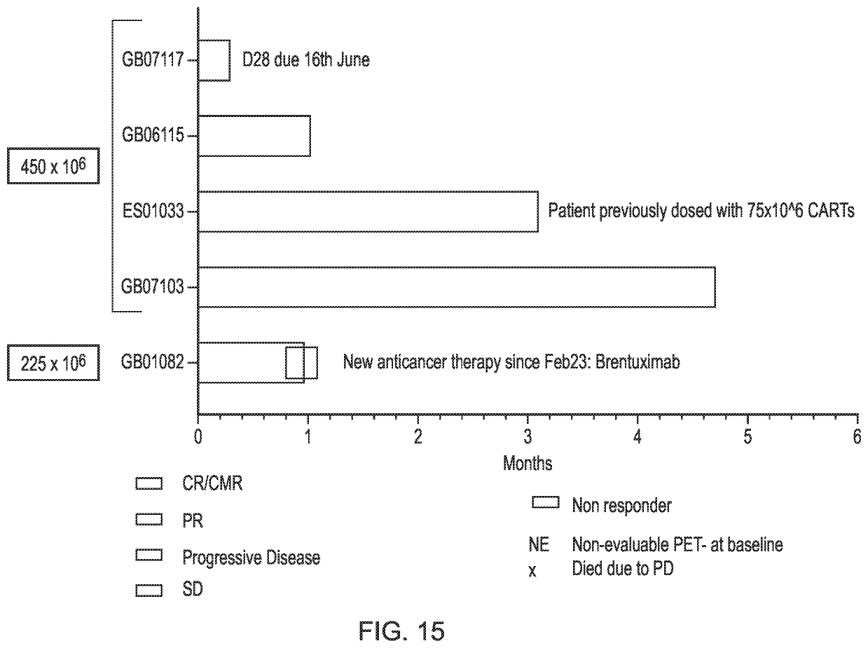

The treatment efficacy post-AUTO4 infusion was determined and results are shown in FIG. 15. The patient dosed with 225×10⁶ cells showed progressive disease at day 28 (D28). Out of the two patients dosed with 450×10⁶ cells, it was too early for tumour assessment in one of them while the other showed stable disease (SD) at D28 and 2 months. Overall, this PTCL-NOS patient health's conditions had drastically improved, with LDH and CRP levels within normal range. Of note, the patient had gone back to work.

Two additional patients were dosed at 450×10⁶ cell dose. However, it was too early for tumour assessment in one of them. The other patient was dosed at the time of progressive disease (PD), which was showed 18 months after having been previously dosed with 75×10⁶ cells with manufacturing Process A.

Since the follow-up period was very short, the data is inconclusive.

### Treatment Emergent Adverse Events (TEAE) Post AUTO4 Infusion, Irrespective of Causality

As shown in Table 8 below, no dose limiting toxicities were observed. There were a low number of CRS and no ICANS.

TABLE 8

| | All infused patients (N = 13) | |
|---|---|---|
| | Any grade, | Grade ≥3, |
| | n, (%) | n, (%) |
| Patients with any TEAE | 12 (92.3) | 12 (92.3) |
| Neutropenia / neutrophil count decreased | 9 (69) | 9 (69) |
| Infections and Infestations | 7 (53.8) | 1 (7.7)* |
| Any Serious TEAE | 5 (38.5) | 4 (30.8) |
| CRS | 7 (53.8) | 1 (7.7)** |
| ICANS | 0 | 0 |

*Cohort 1 (25 × 10\hat{ }6 cells);
**Cohort 4 (450 × 10\hat{ }6 cells)
TEAE, Treatment-emergent adverse events irrespective of causality;
CRS, cytokine release syndrome;
ICANS, Immune Effect Cell-Associated Neurotoxicity Syndrome

### Examples 1-18 Summary

AUTO4 treatment was well tolerated with no DLT. Ongoing CMR in two patients at 15- and 18-months post-dosing at the highest dose tested (450×10⁶) are important results. Moreover, at a median follow-up of 13.8 months, 8/10 (80%) of the patients are alive at last follow-up. The absence of CARs in blood and their detection in lymph nodes suggests fast homing of CARs to tumour sites.

### Schedule of Assessments 1 (Safety and Efficacy Follow-Up)

| | | | | | PRE-CONDITIONING | | | |
|---|---|---|---|---|---|---|---|---|
| | | | LEUKA- | | CHEMOTHERAPY | | TREATMENT STAGE† | |
| | | SCREEN 1^{$} | PHERESIS^{@} | SCREEN 2 ^{#} | | Day −6, | | Day 1, then q.a.d. |
| | SCREEN | Day −84 | Before | Day −35 | Day −7 | −5, −4, −3 | | Hospitaliza- |
| | TRBC | to Day −35 | Day −35 | to −7 | (−1 d) | (−1 d) | Day 0 | tion ≥14 d |
| Informed Consent | Part A | Part B | | | | | | |
| Demographic Data^{[1]} | X | | | | | | | |
| Eligibility Criteria^{[2]} | X | X | | | X | | X | |
| Medical/Lymphoma | X | | | | X | | | |
| History^{[3]} | | | | | | | | |

Examinations/Investigations

| | | | | | | |
|---|---|---|---|---|---|---|
| ECOG Performance | X | X | | X | | |
| Status | | | | | | |
| Physical | | X | | X | X | X^{D 7, D 14} |
| Examination^{[4]} | | | | | | |
| Weight | | X | X | X | | |
| Vital Signs^{[5]} | | X | | X | X^{[5]} | X^{D 7, D 14} |
| 12-lead ECG^{[6]} | | X | | X^{[6]} | | |
| ECHO or MUGA^{[7]} | | X | | | | |

Tumour Tissues Assessment

| | | | |
|---|---|---|---|
| Tumour Tissue | X | | |
| Sample (TRBC1 | | | |
| status) ^{[8]} | | | |
| Tumour Tissue | | | X^{between D 7-D 21 [9]} |
| Sample (CAR T-cell | | | |
| persistence/TRBC1 | | | |
| expression)^{[9]} | | | |
| Bone Marrow Biopsy | | X | |
| (if applicable) ^{[10]} | | | |

Disease Evaluations

| | |
|---|---|
| CT Imaging (neck, | X |
| chest, abdomen and | |
| pelvis) ^{[11]} | |
| 18-FDG-PET Scan | X |
| (skull base to the | |
| proximal femur) ^{[11]} | |
| Tissue biopsy | |

Safety Labs

| | | | | | | |
|---|---|---|---|---|---|---|
| Haematology^{[12]} | X | X | | X | X | X^{D 1, D 7, D 14} |
| Biochemistry ^{[13]} | X | X | | X | X | X^{D 1, D 7, D 14} |
| Ferritin, C reactive | | | | X | X | X |
| protein^{[14]} | | | | | | |
| Coagulation^{[15]} | X | X | | X | X | X^{D 7, D 14} |
| Infectious Disease | X | X^{[16]} | | | | |
| Screen^{[16]} | | | | | | |
| Monitoring for | | | X | | | |
| infections^{[17]} | | | | | | |
| Pregnancy Test^{[18]} | X | | | X | X | |

Pharmacokinetics, Pharmacodynamic and Biomarker Assays

| | | | | | | |
|---|---|---|---|---|---|---|
| Serum for | | X | X | X^{D -6} | X^{p} | X |
| cytokines^{[19]} | | | | | | |
| Central Assessment- | X | | X | | X | X^{D 14 only} |
| Blood for Tcell | | | | | | |
| Subset (TRBC1+, | | | | | | |
| TRBC1−, CD3+ | | | | | | |
| CD4+, CD3+ | | | | | | |
| CD8+) Analysis ^{[20]} | | | | | | |
| Local Assessment- | X | | X | | X | X^{D 14 only} |
| Blood for Tcell | | | | | | |
| Subset (CD3+CD4+, | | | | | | |
| CD3+CD8+) | | | | | | |
| Analysis ^{[20]} | | | | | | |
| Blood for CAR | | | X | | X^{p [21]} | X ^{[21]} |
| T Cells persistance, | | | | | | |
| PCR ^{[21]} | | | | | | |
| Blood for CAR T | | | X | | X^{p} | X |
| Cells, Flow ^{[21]} | | | | | | |
| Blood for RCR | | | X | | | |
| Testing & Insertional | | | | | | |
| Mutagenesis | | | | | | |
| Immunological/ | | | X | | X^{p} | X^{D 7, D 14} |
| genomic profiling^{[22]} | | | | | | |

Treatments

| | | |
|---|---|---|
| CY and FLU | X | |
| AUTO4 Infusion | | X |

Adverse events

| | | | | | | | | |
|---|---|---|---|---|---|---|---|---|
| Adverse Events^{[23]} | X | X | X | X | X | X | X | X |
| Concomitant | | | | | X | X | X | |
| Medication^{[24]} | | | | | | | | |

| | TREATMENT STAGE† | | FOLLOW-UP STAGE | | |
|---|---|---|---|---|---|
| | Weekly | End of | Month 2, 3, 4, | Every 6 | |
| | after | DLT period* | 5, 6, 9, 12, | months | |
| | discharge | Day 28 | 15, 18 & 24 | until EoS** | END OF |
| | (±2 d) | (±3 d) | (±7 d) | (±4 wk) | STUDY** |
| Informed Consent | | | | | |
| Demographic Data^{[1]} | | | | | |
| Eligibility Criteria^{[2]} | | | | | |
| Medical/Lymphoma | | | | | |
| History^{[3]} | | | | | |

Examinations/Investigations

| | | | | |
|---|---|---|---|---|
| ECOG Performance | | X | X | X |
| Status | | | | |
| Physical | X | X | X | X |
| Examination^{[4]} | | | | |
| Weight | | X | X | X |
| Vital Signs^{[5]} | X | X | X | X |
| 12-lead ECG^{[6]} | | | | |
| ECHO or MUGA^{[7]} | | | | |

Tumour Tissues Assessment

| Tumour Tissue | | | |
|---|---|---|---|
| Sample (TRBC1 | | | |
| status) ^{[8]} | | | |
| Tumour Tissue | X^{between D 7-D 21 [9]} | | X^{prog} |
| Sample (CAR T-cell | | | |
| persistence/TRBC1 | | | |
| expression)^{[9]} | | | |
| Bone Marrow Biopsy | | X¹⁰ | |
| (if applicable) ^{[10]} | | | |

Disease Evaluations

| | | | | |
|---|---|---|---|---|
| CT Imaging (neck, | X | X^{M 3, M 6, M 9, M 12, M 15, M 18, M 24} | X | [11] |
| chest, abdomen and | | | | |
| pelvis) ^{[11]} | | | | |
| 18-FDG-PET Scan | X | X^{M 3, M 6, M 9, M 12, M 15, M 18, M 24} | X | [11] |
| (skull base to the | | | | |
| proximal femur) ^{[11]} | | | | |
| Tissue biopsy | | ^{[11]} | ^{[11]} | |

Safety Labs

| | | | |
|---|---|---|---|
| Haematology^{[12]} | X | X | X |
| Biochemistry ^{[13]} | X | X | X |
| Ferritin, C reactive | X | X | |
| protein^{[14]} | | | |
| Coagulation^{[15]} | | | |
| Infectious Disease | | | |
| Screen^{[16]} | | | |
| Monitoring for | | X | X |
| infections^{[17]} | | | |
| Pregnancy Test^{[18]} | | X | X^{M 3, M 6, M 12} |

Pharmacokinetics, Pharmacodynamic and Biomarker Assays

| | | | | |
|---|---|---|---|---|
| Serum for | X | X | X^{M 2, M 3} | |
| cytokines^{[19]} | | | | |
| Central Assessment- | | X | X^{M 2, M 3, M 4, M 5, M 6, M 9, M 12, M 15, M 18, M 24} | |
| Blood for Tcell | | | | |
| Subset (TRBC1+, | | | | |
| TRBC1−, CD3+ | | | | |
| CD4+, CD3+ | | | | |
| CD8+) Analysis ^{[20]} | | | | |
| Local Assessment- | | X | X^{M 2, M 3, M 4, M 5, M 6, M 9, M 12, M 15, M 18, M 24} | X |
| Blood for Tcell | | | | |
| Subset (CD3+CD4+, | | | | |
| CD3+CD8+) | | | | |
| Analysis ^{[20]} | | | | |
| Blood for CAR | X | X | X^{M 2, M 3, M 4, M 5, M 6, M 9, M 12, M 15, M 18, M 24} | X |
| T Cells persistance, | | | | |
| PCR ^{[21]} | | | | |
| Blood for CAR T | X | X | X^{M 2, M 3, M 4, M 5, M 6, M 9, M 12, M 15, M 18, M 24} | X |
| Cells, Flow ^{[21]} | | | | |
| Blood for RCR | | X | X^{M 3, M 6, M 12, M 18, M 24} | X |
| Testing & Insertional | | | | |
| Mutagenesis | | | | |
| Immunological/ | X | X | X^{M 3, M 6, M 9, M 12, M 15, M 18, M 24} | |
| genomic profiling^{[22]} | | | | |

Treatments

CY and FLU
AUTO4 Infusion

Adverse events

| | | | | | |
|---|---|---|---|---|---|
| Adverse Events^{[23]} | X | X | X | X | X |
| Concomitant | | X | X^{M 2} | X | |
| Medication^{[24]} | | | | | |

Abbreviations: AE = adverse event; ALT = alanine aminotransferase; AST = aspartate aminotransferase; CAR = chimeric antigen receptor; CMV = Cytomegalovirus; CPK = creatine phosphokinase; CR = complete response; CRS = cytokine release syndrome; CT = computed tomography; CY = cyclophosphamide; D, d = day; DLT = dose limiting toxicity; EBV = Epstein Barr Virus; ECG = electrocardiogram; ECOG = Eastern Cooperative Oncology Group; ECHO = echocardiogram; EDTA = ethylenediaminetetraacetic acid; FDG = fluorodeoxyglucose; FFPE = formalin fixed paraffin embedded; FLU = fludarabine; JCV = John Cunningham Virus; HHV6 = Human herpesvirus 6; M = month (where each month is approximately 4.2 weeks, with 12 months per year); MRI = magnetic resonance imaging; MUGA = multigated acquisition (cardiac scan); PET = positron emission tomography; q.a.d. = quaque altera die (every other day); RCR = replication competent retrovirus; SAE = serious adverse event; TRBC = T cell receptor beta constant.
*End of DLT period set as 28 days after the dose of AUTO4.
**End of Study visit is to be performed upon completion of all other study visits or in case of premature withdrawal. The end of the study (EoS) is defined as the LPLV expected to be 24 months after the last treated patient with AUTO4 or earlier in the event of patient death or consent withdrawal. Of Note: The patients who experienced disease progression post AUTO4 infusion will continue to be followed under this study protocol until death, study closure or consent withdrawal, whichever occurs earlier (see Section 8.5.2, Schedule of Assessments).
Note:
Additionally, blood samples for CAR T persistence and RCR may also be collected.
^{$}All tests must be undertaken (and results known) before leukapheresis.
^{@}Leukapheresis occurs after a patient is confirmed as TRBC1 positive.
X^{Dx} Test to be performed on a particular day or month of the schedule rather than systematically at every visit. Please refer to the number to determine the day or month of assessment.
X^{p} Sample to be taken prior to infusion.
X^{prog} Test to be performed at disease progression.
^{#} Enrolment confirmed once all inclusion and exclusion criteria have been fulfilled and leukapheresate has been accepted for manufacturing.
Schedule of Assessment Footnotes:
^{[1]}Demographic data: race and ethnicity, height, age (month and year) and gender.
^{[2]}Eligibility criteria: Performance, disease characteristics and organ and bone marrow function to be assessed before a new node biopsy (if patient will undergo a node biopsy). ECHO may be done after node biopsy. Eligibility criteria to be re-assessed on Day -7 (-1 day) prior to pre-conditioning when the patient should continue to meet renal, hepatic, pulmonary function and performance status requirements. On Day 0, before infusion, it will be assessed whether the patient meets the AUTO4 infusion criteria.
^{[3]}Medical/lymphoma history: to include all current and prior clinically significant diseases, surgeries, cancer history (including prior T-cell lymphoma therapies or any other cancer therapies and procedures) and prior relevant medications). Obtain histological confirmation of disease diagnosis (pathology report) and the presence of T-cell lymphoma in the archived node tissue (if archived tissue is used for the TRBC1 status assessment). Record disease status at Screen 2 after leukapheresis has been completed.
^{[4]}Physical examination: a complete physical examination and complete neurological examination to be performed at Screen 1, Day −7 (−1 day) and Day 0; then focused and/or symptom related examination as appropriate at following visits.
^{[5]}Vital signs: temperature, systolic and diastolic blood pressure, pulse/heart rate, oxygen saturation and respiratory rate will be performed while the patient is in a seated position or supine. On Day 0 of any treatment stage, record vital signs immediately prior to AUTO4 infusion and every 30 minutes (±10 min) for the next 4 hours post AUTO4 infusion, and thereafter monitored as per hospital policy but no less than 3 times a day whilst the patient is in hospital. Record weight as per Schedule of Assessments above.
^{[6]}12-lead ECG: Repeat as clinically necessary and when patient experiences CRS.
^{[7]}ECHO or MUGA cardiac scan: to be performed at Screen 1 and to be repeated if clinically indicated. Same method should be used throughout the study.
^{[8]} Newly acquired tumour tissue sample may be required to determine TRBC1 status unless sufficient archival tumour biopsy material can be obtained - either by core needle biopsy or excisional biopsy (archived tissue must not be >5 years old and subtype of T-NHL unchanged from time of archived tissue to current status). If a core needle is used, an absolute minimum of two cores are required for the evaluation of TRBC1 expression on T cells using the LymphoTrack Dx TRB Assay. However, additional two cores are requested (if medically feasible) for the further development of a TRBC companion diagnostic assay and/or biomarker assessment on FFPE tissue.
^{[9]}Lymph node Tumour Tissue Sample for CAR T-cell persistence/TRBC1 expression. If there is a suitable lesion in an accessible location which would not put the patient at any safety risk per treating physician judgment. Biopsy samples should be taken once within the first 10 days since CAR T cell infusion and at the time of progression. The tumour samples will be analysed by flow cytometry or immunohistochemistry.
^{[10]} Bone Marrow Biopsy. If the Investigator suspects there is lymphoma infiltration in the bone marrow, a bone marrow biopsy should be performed at screening (if patient receives bridging therapy it should be done after any bridging therapy). If a bone marrow biopsy is performed and shows lymphoma infiltration, a bone marrow biopsy should be repeated at the time of first complete response.
^{[11]} Imaging and scans: For those patients receiving a bridging chemotherapy regimen, the baseline PET/CT (CT portion needs to have diagnostic quality, otherwise a separate CT is needed to be taken in the same week of PET) scans must be done after completion of bridging therapy and before start of the preconditioning and AUTO4 infusion. ¹⁸-FDG-PET Scan: If at 6 months the patient has a CMR on PET scan, CT scans alone may be used for future assessment timepoints, if clinically appropriate. If relapse occurs after CMR or disease progression is suspected (e.g. new or enlarging lesion(s) detected on CT scan), a PET scan is to be repeated to confirm relapse/progression together with tissue biopsy (if needed). MRI may be used to evaluate sites of disease that cannot be adequately imaged using CT (in cases where MRI is desirable, the MRI must be obtained at baseline and at all subsequent response evaluations). For all other sites of disease, MRI studies do not replace the required neck, chest, abdomen, and pelvic CT scans. Brain MRI is only required if clinically indicated. If progression is suspected from scan(s), but the patient is otherwise not showing clinical progression/deterioration, the disease progression must be confirmed not less than 28 days after initial finding to rule out a pseudo-progression. In cases of starting new treatment during response, an efficacy assessment is required prior to new treatment.
^{[12]}Haematology: haemoglobin, red blood cell count, platelet count, white blood cell count with differential (neutrophils, eosinophils, lymphocytes, monocytes, and basophils). Test to be performed prior to chemotherapy on pre-conditioning days and prior to AUTO4 infusion on Day 0 of any treatment stage.
^{[13]} Biochemistry: Whole panel: sodium, phosphate, potassium, magnesium, chloride, bicarbonate, ALT, AST, urea or blood urea nitrogen, creatinine, serum CPK, lactate dehydrogenase, glucose, total bilirubin, calcium (albumin adjusted), total protein, albumin. Serum uric acid to be measured only on Day 0, 1, and 7 of any AUTO4 treatment stage. All tests must be performed prior to AUTO4 infusion on Day 0 of any AUTO4 treatment stage. Glomerular filtration rate should be calculated at screening as per institutional preferred method.
^{[14]}Ferritin, C reactive protein: May be done more frequently as clinically necessary and during CRS if necessary.
^{[15]}Coagulation: prothrombin time, international normalised ratio, activated partial thromboplastin time, fibrinogen. Day 7, after AUTO4 infusion.
^{[16]}Infectious disease screen: must be performed at screening for the eligibility criteria and within 30 days prior to leukapheresis and must be negative. It must be repeated on the day of leukapheresis (or within 7 days after). HIV-1 and 2, Hep B virus, Hep C virus, HTLV-1, HTLV-2, Syphilis.
^{[17]}Monitoring for infections: CMV, HHV6, EBV & adenovirus monitoring as per schedule in table. Additonal monitoring for opportunistic infections, such as JCV, toxoplasmosis and fungal infections as per institutional guidelines (e.g institutional guidelines used for bone marrow transplant patients) or as clinically indicated. Monitoring beyond 3 months should be done if there is low levels of CD4+ T-cells or if clinically indicated.
^{[18]}Pregnancy test: serum (B-human chorionic gonadotropin) or urine pregnancy testing for women of childbearing potential.
^{[19]}Serum for cytokines and biomarkers: During hospital stay, sample collection to be performed every other day (±1 day). If patient experiences ≥Grade 2 CRS then additional samples should be collected daily until CRS resolves or clinically indicated
^{[20]} Blood for analysis of T cell Subsets will be done both locally and via a Central Lab. Samples are to be collected at the following timepoints: Screening, Day −7, Day 0 (predose), Day 14, Day 28, Month 2, 3, 4, 5, 6, 9, 12, 15, 18, 24, and as clinically indicated e.g. in case of opportunistic infections
^{[21]} Blood for CAR T cells persistance: one sample to be taken on Day −7, Day 0 prior to AUTO4 infusion. During hospital stay, sample collection to be performed 10 min and 1 h after completion of CAR T cell infusion, on Day 1 and every other day (±1 day) and ideally on a Monday to Friday. Following hospital discharge (Day 14), blood samples for CAR-T cells (by flow and PCR) are to be collected at the following timepoints: Day 21, Day 28, Month 2, 3, 4, 5, 6, 9, 12, 15, 18 and 24. Additional samples should be collected if clinically indicated; an additional sample should be collected at the time of disease progression.
^{[22]}Blood for Immunological/genomic profiling: During hospital stay, sample collection to be performed on Day 7 and Day 14 (±1 day) and ideally on a Monday to Friday
^{[23]}Adverse Events: Only AEs/SAEs related to study procedures should be collected until admission for lymphodepletion chemotherapy (Day −6 [−1 day]). AEs related to intervening/bridging non-study related anti-cancer therapy administered prior to pre-conditioning or AEs associated with disease progression during the same period will not be reported as AEs. These events will be recorded as an update to the patient's medical history. After Day 60, only collect: All SAES and AUTO4 treatment-related non-serious AEs; All AEs of special interest and AEs related to a study procedure. Please refer to Section 12 for specifics about the AE reporting periods.
^{[24]}Concomitant medications: Collect as described in the Schedule of Assessments. Before Day −7 (−1 day) and after 2 months, collect only concomitant medications relevant to AUTO4 treatment-related Grade 3 to 4 AEs and treatment-related SAEs; AEs of special interest or AEs related to a study procedure.
Note:
The total estimated volume of blood collected for safety, biomarkers and immunological assessments (with the exception of the leukapheresis procedure) across any one year will not normally exceed 850 mL (this is expected volume for females with serum pregnancy test). The maximum volume of blood collected on any day will unlikely exceed 80 mL. No more than 470 mL of blood will be collected in any 28-day period.

### Example 19: Clinical Study—Longer Follow-Up, Materials and Methods

In this present study, the preliminary safety and efficacy CAR-T cell product specifically targeting TRBC1+ tumours in r/r PTCL using AUTO4, an autologous CAR T cell therapy based on Jovi-1, was explored.

### TRBC1 Screening

TRBC1 status could be determined either by immunohistochemistry or using the commercially available and CE IVD marked LymphoTrack Dx TRB Assay (Invivoscribe, USA).

Assessment of TRBC1 expression on malignant cells by immunohistochemistry was performed on serial sections (5 μm thickness) of fresh frozen lymph node tissue biopsies collected for screening. Single-staining was performed for the following antibody markers: TRBC1 (JOVI-1 murine IgG22 monoclonal antibody clone, GeneTex, USA), TCR VßF1 (Clone 8A3, GeneTex, USA) and Ki-67 (M1B-1, Leica Biosystems, UK). TCR Vß F1 staining enables identification of T cells, whist Ki-67 allows for the distinction between healthy and proliferating malignant T-cells. Staining was performed using the OptiViewDAB IHC detection kit (Ventana, USA) on the BenchMark ULTRA DISCOVERY automated platform (Ventana). The tumour was considered TRBC1 clonal if ≥40% of viable tumours cells exhibit membrane staining at any intensity (≥1+) as reviewed by an expert hematopathologist.

The LymphoTrack Dx TRB Assay was performed at the Laboratory of Personalized Molecular Medicine, Invivoscribe (Hallbergmoos, Germany) according to manufacturer's instructions to enable prospective patient selection. Briefly, DNA was extracted from 6 to 15 curls (5 μM thickness) from FFPE blocks and PCR amplified using 24 illumina indexed master mixes with proprietary primer sets within Vβ and Jβ regions, before NGS using a MiSeq instrument. The sequencing data was analyzed using the LymphoTrack Dx Software-MiSeq package (Invivoscribe). The Merged Read Summary Report was used to identify the top merged read sequences and their frequencies to assess clonality. Evidence of clonality was determined if top merged read represented ≥2.5% (if ≥20000 total reads) or ≥5% (if ≥10000 or <20000 reads) of the total reads, and if the top merged read was >2× the % of the 5th most frequent merged sequence for a detected D-J rearrangement, or >2× the % of the 3rd most frequent merged sequence for a detected J rearrangement. Clonal incomplete V-J sequences, as opposed to D-J sequences, were not considered acceptable to determine eligibility. Usage of J1 or J2 in rearranged sequences was used to infer TRBC1 or TRBC2 association, respectively.

### Vector and Vector Manufacture

AUTO4 is an autologous CAR T-cell product co-expressing a humanized second-generation CAR targeting TRBC1 and the RQR8 safety switch, achieved by transduction of TRBC2 positive cells with a single bicistronic γ-retroviral vector. The TRBC1 CAR was constructed from a scFv derived from a humanized form of the JOVI-1 antibody fused to CD8a stalk fused to the endodomains of 41BB and CD3ζ (FIG. 7). RQR8 is a fusion of two copies of a rituximab binding mimotope separated by a fragment of human CD34 which allows selective depletion of transgenic T cells with the therapeutic mAb rituximab in the event of unmanageable toxicity. In addition, RQR8 allows convenient tracking and selection of CAR T cells by staining with the anti-CD34 antibody QBEnd10 mAb. γ-retroviral vector (γRV) was produced under Good Manufacturing Practice (GMP) conditions by 3-plasmid co-transfection of HEK293T cells and subsequent harvest and purification of the culture supernatant4. The viral vector was pseudotyped with the RD114 envelope.

### CAR T Cell Manufacture

CAR-T production was performed on the Miltenyi CliniMACS Prodigy™ with autologous leukapheresate used as starting material. First, pheresate was incubated with biotinylated anti-TRBC1 (using JOVI-1 mAb) antibody for 10 minutes at 4° C. followed by a wash step and secondary labelling with an anti-biotin CliniMACS. TRBC1 depletion was then performed with the MACS column on the CliniMACS Prodigy. The TRBC1+ depleted cells were washed and resuspended in TexMACS with 3% HABS and activated with TransAct as per manufacturer's instructions. On day 2, cells were transduced with γRV facilitated with VectoFusin (Vf) and using spinoculation at 400 g for 2 hours at 32° C. on the Prodigy. On Day 3, the cells were washed and expanded up to Day 10. Cells were then cryopreserved in one or more CryoMACS® bag(s) and stored in a vapour-phase liquid nitrogen environment prior to administration.

### Flow Cytometry Leukapheresis and Drug Product Characterisation

Frozen drug product and Leukapheresis characterization experiments included fluorescent minus multiple controls (FMX), PBMC and single stained UltraComp eBeads™ Compensation Beads (ThermoFisher Scientific, UK) to determine gating thresholds and calculate compensation. Samples were rested overnight in TexMACS10 medium (Miltenyi Biotec) post thaw. Samples were stained with a Fixable viability dye (BD Horizon™) and then blocked (Miltenyi Biotec). Phenotypic Characterization was performed using antibodies for memory and exhaustion markers diluted in Brilliant Stain Buffer Plus (BD Horizon™). Staining for CCR7 was carried out prior to surface staining at 37° C. for 15 mins (Biolegend®). Intracellular staining was done using the Transcription Factor Buffer Set (eBioscience™) according to manufacturer's instructions. Transformed CAR cells were identified using Anti-RQR8 antibody conjugated to PE (R&D Systems, USA). Samples were analysed using FCS Express software (De Novo software, US). Details of the antibodies are shown in Table 10.

TABLE 10

| Laser | Filter | Fluor | Antigen | Clone | Cat no | Supplier | Description |
|---|---|---|---|---|---|---|---|
| UV | UV | BUV395 | HLA DR | G46-6 | 564040 | BD | Late activation |
| | 379/28 | | | | | | |
| | UV | BUV496 | CD8 | RPA-T8 | 612942 | BD | Subset |
| | 515/30 | | | | | | |
| | UV | BUV737 | CD27 | M-T271 | 741833 | BD | Memory |
| | 740/35 | | | | | | phenotype |
| | UV | BUV805 | CD4 | SK3 | 612887 | BD | Subset |
| | 805/60 | | | | | | |
| Violet | V 431/28 | BV421 | CXCR3 | 1C6/CXCR3 | 562558 | BD | Trafficking/ |
| | | | | | | | Upregulated on |
| | | | | | | | activation |
| | V 470/20 | BV480 | CD45RA | HI100 | 566114 | BD | Memory |
| | | | | | | | phenotype |
| | V 610/20 | BV605 | CD2 | RPA-2.10 | 300224 | Biolegend | — |
| | V 670/30 | BV650 | CD62L | DREG-56 | 563808 | BD | Memory |
| | | | | | | | phenotype |
| | V 710/50 | BV711 | TIM3 | 7D3 | 565566 | BD | Checkpoint |
| | | | | | | | inhibitor |
| | V 780/60 | BV786 | CD25 | M-A251 | 563701 | BD | Intermediate |
| | | | | | | | stage activation |
| Blue | B 530/30 | FITC | LAG3 | 11C3C65 | 369308 | BD | Checkpoint |
| | | | | | | | inhibitor |
| | B 710/50 | PerCP | Tigit | MBSA43 | 46- | Invitrogen | Checkpoint |
| | | eFluor710 | | | 9500- | | inhibitor |
| | | | | | 42 | | |
| Yellow | YG | PE | Anti- | RQR8 | | | |
| | 586/15 | | CAR | | | | |
| | YG | PE-Dazzle | CCR7 | G043H7 | 353236 | Biolegend | Memory |
| | 610/20 | | | | | | phenotype |
| | YG | Pe-Cy7 | PD-1 | EH12.1 | 561272 | BD | Checkpoint |
| | 780/60 | | | | | | inhibitor |
| Red | Red | APC | CD3 | SK7 | 345767 | BD | — |
| | 670/30 | | | | | | |
| | Red | AF700 | GrB | GB11 | 560213 | BD | Cytotoxic T cells |
| | 730/45 | | | | | | |
| | Red | FVS780 | L/D | | 565388 | BD | Live discriminator |
| | 780/60 | | | | | | |

### Study Design

A multi-centre, single-arm clinical study of AUT04 in r/r TRBC1-positive PTCL was carried out. The study consisted of two phases: Phase I dose escalation and Phase II dose expansion. Each patient went through the following five steps: 1) Screening stage consisting of TRBC1 screening and eligibility inclusion/exclusion criteria assessment as detailed in Example 4 and Example 5; 2) Leukapheresis stage followed by AUT04 manufacture; 3) Pre-conditioning stage consisting of lymphodepleting treatment with fludarabine (30 mg/m² on Days −6 to −3) and cyclophosphamide (500 mg/m² on Days −6 and −5) (Flu/Cy) prior to AUTO4 infusion; 4) Treatment stage in which AUTO4 is administered i.v. as a single infusion on Day 0; 5) Follow-up stage starting after AUTO4 administration up to 24 months after the infusion of the last patient with AUTO4 or at their disease progression or withdrawal of consent.

In Phase I dose escalation four dose levels were explored: Cohort 1 (n=3 patients) received 25×10⁶ AUTO4 T-cells; Cohort 2 (n=2 patients) received 75×10⁶ AUTO4 T-cells; Cohort 3 (n=1 patient) received 225×10⁶ AUTO4 T-cells and Cohort 4 (n=4 patients) received 450×10⁶ AUTO4 T-cells.

Patients were assigned sequentially to dose groups, with a rolling 6 design. For Cohorts 2 and 3, if no CAR-T expansion was detected in any of the patients treated (with at least one patient treated at that dose), together with no Grade 1 CRS/Neurotoxicity or Grade 2 AUTO4-related adverse events in the DLT period (first 28 days after AUTO4 infusion) accelerated escalation to the next level was allowed. If CAR-T expansion was detected in the potential single patient cohorts (Cohort 2 and 3) the cohort must be expanded to a minimum of 3 patients treated. For cohort 4, the standard rolling six design was applied, with a minimum of 3 patients treated.

Primary endpoints in Phase I were incidence of Grade 3 toxicity occurring within 60 days of AUTO4 infusion and the frequency of dose limiting toxicities (DLT) within 28 days of AUTO4 infusion. Overall response (CR+PR) rate by Lugano PET-CT criteria was a secondary endpoint (Example 13 and Example 14).

Data were locked as of 8^{th} February 2023.

### Toxicity Assessment

1. Adverse events over the first 28 days post-CAR infusion were graded as per Common Terminology Criteria for Adverse Events (version 5.0).
2. CRS and neurotoxicity were graded by the American Society for Transplantation and Cellular Therapy (ASTCT)/CTCAE v.5.0 and American Society for Blood and Marrow Transplantation (ASBMT).
3. Immune Effector Cell-associated Neurotoxicity Syndrome (ICANS) were graded according to grading criteria by Lee et al., 2019 [Biol. Blood Marrow Transplant. 25, 625-638 (2019)].
4. Hemophagocytic lymphohistiocytosis (HLH) was graded as per Neelapu et al, 2018 [Nat. Rev. Clin. Oncol. 15, 47 (2018)].

### Response Assessment and Translational Analysis

Disease response assessments were performed at protocol defined time points (pre-LD, months 1, 3, 6, 9, 12, 15, 18, 24) by 18FDG PET-CT according to the Response Criteria for Non-Hodgkin Lymphoma-Lugano Classification [Cheson, B. D. et al. J. Clin. Oncol. Off J. Am. Soc. Clin. Oncol. 32, 3059-3068 (2014)]. All subjects had disease status evaluation within 4 weeks of initiation of lymphodepleting chemotherapy (LD). For those patients who received a bridging chemotherapy regimen, baseline PET/CT scans was done post-bridging therapy and pre-LD and AUTO4 infusion.

### Whole Blood Flow Cytometry

The surface assay follows a lyse wash protocol, for each sample an FMO control was generated to determine CAR positivity. The compensation was set up using the Lyric software reference settings maintained daily by using the performance QC wizard. When creating reference settings single stained compensation beads (BD Horizon™ BD Biosciences, USA) were used. A volume of 100 μL of whole blood was used for staining. The sample was blocked (Miltenyi Biosciences, Germany) followed by staining with a cocktail of antibodies to identify the immune cell subsets, and the presence of transformed CARs.

To assess viability, a fixable viability dye (BD Horizon™) was added to Lysis solution (BD Biosciences). The Live/Lysis solution was added to the blood and incubated. Cells were washed and resuspended in BD Stain buffer (BD Biosciences).

The sample was then transferred to a TruCount™ Tube (BD Biosciences) for acquisition and analysis on the BD FACSLyric (BD Biosciences).

TRBC1/TRBC2 ratio was determined using a surface assay following a two-step staining protocol using Fluorescence minus one controls and secondary control for TRBC2. Frozen PBMCs were thawed, blocked using Human FcR Block (Miltenyi Biotec) and stained for viability using Fixable Viability Stain 700 (BD Biosciences). Cells were washed and resuspended in a surface stain master mix containing anti-CD45 PerCP-Cy 5.5 (332784, BD), anti-CD3 BV510 (300448, Biolegend), anti-CD4 BV605 (344646, Biolegend), anti-CD8 PE-Cy7 (335822, BD), anti-CD19 BV786 (563325, BD), anti-TCR beta 1 AF488 (Santa Cruz Biotechnology, US), CD34 PE (FAB7227P, R&D systems) and anti-TRBC2 biotin (PGNKWGR, produced in-house). Cells were washed and resuspended in a secondary only master mix containing Streptavidin antibody BV421 (405225, Biolegend). Cells were washed, resuspended, and results acquired BD FACSLyric (BD Biosciences). Data was analysed using FCS Express (De novo software).

### CAR T Persistence by PCR

DNA was extracted from whole blood samples using the QIAamp DNA Mini Kit (Qiagen, Hilden, Germany) according to manufacturer's instructions. Extracted DNA was diluted to a desired final concentration and used as template in a ddPCR reaction. Each reaction contains HindIII-HF DNA digest along with primers and probes to detect both the target Psi packaging gene and the reference gene RPP30 to determine vector copy numbers. Following reaction set up, in each well, the ddPCR reaction was partitioned into nanolitre sized water-in-oil droplets using the QX200 Droplet Generator (Bio-Rad, USA) and subsequently amplified by PCR. Positive and negative droplets were then quantified on the QX200 Droplet Reader Generator (Bio-Rad), Using the QuantaSoft software Generator (Bio-Rad), Poisson statistics are applied to the data to determine the target copy number variation of Psi and normalization against the two-copy RPP30 values yields vector copy number (VCN) per cell.

Lymph Node FFPE ddPCR

DNA was extracted from fresh frozen FFPE Embedded Lymph node tissue biopsy curls (3×10 μM) using the QIAamp DNA FFPE Tissue Extraction KIT (Qiagen, Germany), as per manufacturer's instructions. Extracted DNA was diluted to desired final concentration and used as template for ddPCR reaction. For the ddPCR assay please refer to Whole Blood ddPCR.

### Lymph Node FFPE Multiplexed Immunofluorescence

A 4-colour panel was developed to assess CAR T-cell persistence in FFPE lymph node biopsies (n=5) collected post AUTO4 product infusion. The panel includes the following antibodies: CD34 (QBend10, Leica biosystems, UK) for CAR T-cell detection; CD3 (NCL-L-CD3565, Leica Biosystems, UK) to detect T-cells; K167 (MIB-1, Leica Biosystems, UK) to aid the distinction between healthy and proliferative malignant T-cells; and spectral DAPI (Akoya biosciences, US) for nuclei identification. An Opal 6-Plex Manual Detection Kit (Akoya Biosciences, US) was used to add fluorescent labels to primary antibodies. Staining was performed on the Leica Bond RX platform with the Bond Polymer refine detection kit (Leica Biosystems, UK). Multispectral imaging was performed using the PhenoImager HT and exported from the inForm® software (Akoya biosciences, US). Multispectral whole slide images were generated. Additionally, regions of interest were acquired at a 20× magnification. Image cell segmentation was performed on QuPath on the DAPI channel with the StarDist package for automatic cell detection. CAR persistence was determined as % CAR T-cells (CD3+CD34+) per total T-cells (CD3+).

### Serum Cytokines

Two Meso Scale Discovery (MSD, Rockville, USA) multiplex sandwich immunoassay panels (10-V Plex and Cytokine Human Panel 1), performed at Q2 Solutions (Edinburgh, UK) according to manufacturer's instructions, were used to determine serum cytokines (IFN-γ, GM-CSF, IL-2, IL-5, IL-6, IL-8, IL-10, IL-15, IL-7 and TNF-α) concentrations in serum samples.

### Statistical Analysis

Clinical data are captured in the clinical database via the Encapsia electronic data capture (EDC) system v1.0. SAS 9.4 was used for clinical data analysis. All data are summarized descriptively due to the Phase I exploratory nature of the study.

Categorical variables are reported in terms of frequency and percentage, and continuous variables in terms of median and range unless otherwise specified. Time-to-event outcomes were summarized using the Kaplan-Meier method. Toxicity events are reported at the maximum grade experienced according to the CTCAE.

### Example 20: Clinical Study—Longer Follow-Up, Results

### Patient Characteristics

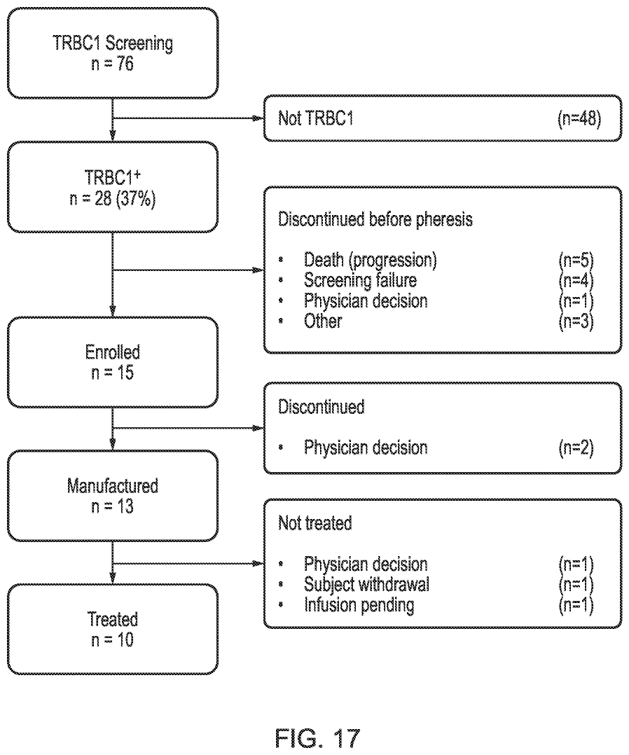

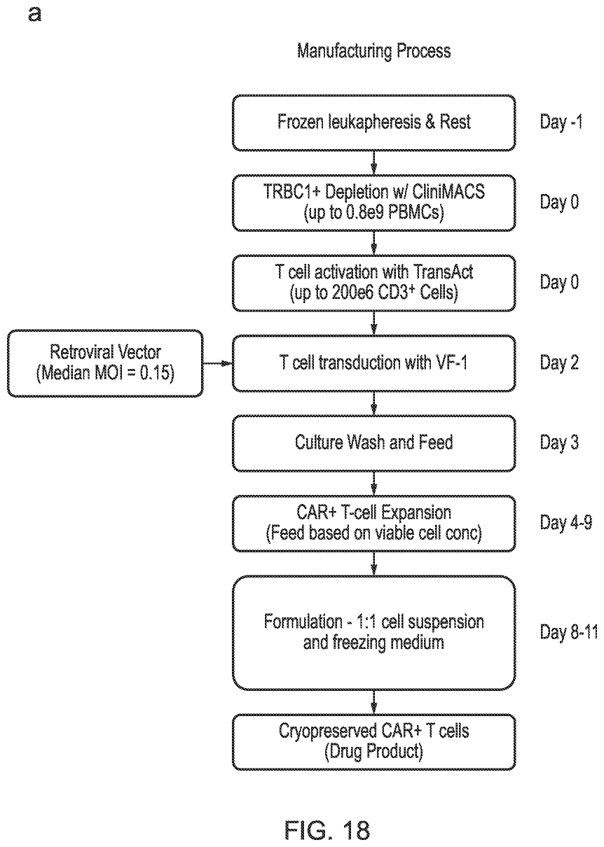

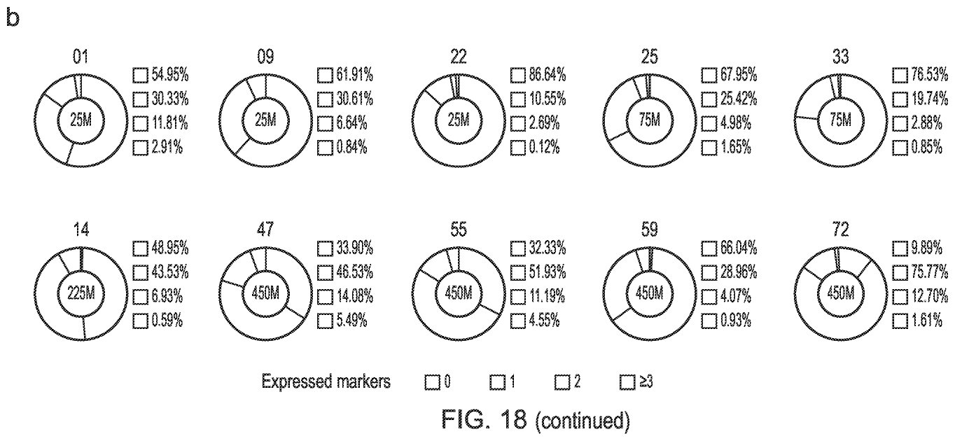

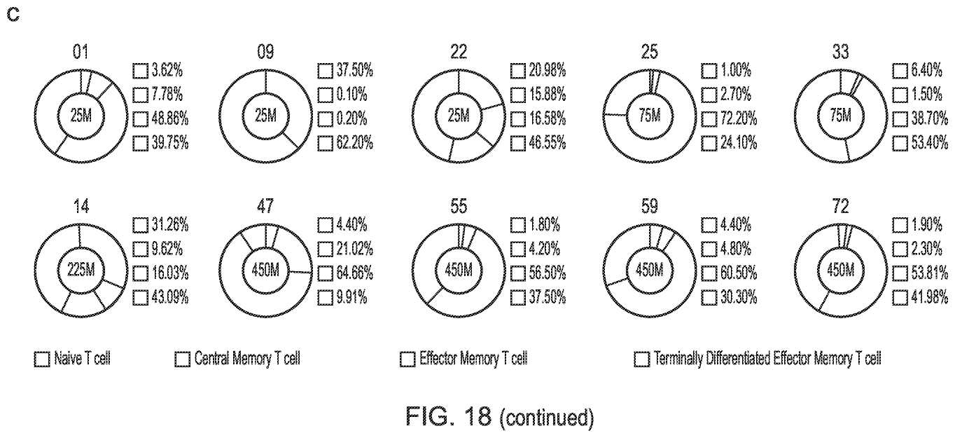

As of 28 Apr. 2023, diagnostic material from 76 patients with relapsed/refractory PTCL (PTCL-NOS, AITL or ALCL) was screened by NGS or IHC. Twenty-eight (37%) were found to be TRBC1+. Subsequent eligibility required FDG-avid measurable disease on PET-CT per Lugano classification and an Eastern Cooperative Oncology Group (ECOG) Performance Status 0 or 1 and lack of central nervous system (CNS) disease. Thirteen patients discontinued before apheresis (5 due to progressive disease; 4 due to screen failure; 2 were outside the allowable screening window for treatment, 1 due to patient/physician choice and 1 due to septic shock/multiorgan failure). Fifteen patients were apheresed and enrolled. AUTO4 was successfully manufactured for 13 patients (manufacture failed in 1 patient and target dose was not reached in another, both patients discontinued due to physician decision). Ten patients were dosed with AUTO4. Patients were dosed in one of 4 cohorts: 25×10⁶, 75×10⁶, 225×10⁶ or 450×10⁶ CAR T cells (FIG. 17). The study allowed rapid single patient dose escalation in the 75 and 225×10⁶ cohorts if no toxicity was observed. Infused patient demographics and disease features are summarized in Table 11. Median patient age was 55 years (range 34 to 63) and 80% had stage III/IV disease. Five patients (50%) had relapsed disease and 5 patients (50%) were refractory to last line of therapy. All patients except for one ALCL patient who achieved CR upon brentuximab bridging (#14, cohort 3) had FOG-avid measurable PET-positive disease prior to Flu/Cy lymphodepletion and AUT04 infusion. Of the infused patients, five were diagnosed with PTCL-NOS, 4 with AITL and with anaplastic lymphoma kinase (ALK)-negative ALCL. Patients received a median of 2 prior lines of therapy (range 1-5), including autologous stem cell transplantation (ASCT) in 3 patients (30%). Bridging therapy was administered in 80% of patients (Table 12). AUT04 drug product characteristics are summarized in Table 13 and FIG. 18.

TABLE 11
Patient characteristics.

| Patient | Cohort | Histologic | | | No. of | Prior | |
|---|---|---|---|---|---|---|---|
| ID | Dose | subtype | Age | Sex | prior lines | ASCT | Bridging |
| 22 | 25 × 10⁶ | PTCL-NOS | 34 | F | 5 | N | Y |
| 01 | 25 × 10⁶ | AITL | 57 | M | 2 | N | Y |
| 09 | 25 × 10⁶ | AITL | 61 | F | 2 | Y | N |
| 33 | 75 × 10⁶ | PTCL-NOS | 35 | F | 1 | N | Y |
| 25 | 75 × 10⁶ | PTCL-NOS | 53 | M | 4 | N | Y |
| 14 | 225 × 10⁶ | ALCL | 47 | M | 3 | Y | Y |
| 72 | 450 × 10⁶ | PTCL-NOS | 44 | M | 2 | Y | Y |
| 55 | 450 × 10⁶ | AITL | 63 | M | 3 | N | Y |
| 59 | 450 × 10⁶ | PTCL-NOS | 58 | M | 3 | N | N |
| 47 | 450 × 10⁶ | AITL | 61 | M | 2 | N | Y |

TABLE 12
Clinical study subjects detailed treatment and bridging history.

| | Cohort | Histologic | | | | |
|---|---|---|---|---|---|---|
| ID | dose | subtype | Age | Sex | IPI | Treatment history and Bridging |
| 22 | 25 × 10⁶ | PTCL | 34 | F | Low- | 5 prior lines of treatment: CHOP, ESHAP, |
| | | NOS | | | Int | brentuximab vedotin, |
| | | | | | | cyclophosphamide/prednisone. The patient did not |
| | | | | | | respond to any prior line. Patient did not have |
| | | | | | | ASCT. |
| | | | | | | Bridging therapy with |
| | | | | | | cyclophosphamide/prednisone with no response. |
| 01 | 25 × 10⁶ | AITL | 57 | M | Low- | 2 prior lines of treatment: CHOP achieving CR for 6 |
| | | | | | Int | months, 2. IEV without achieving response. No |
| | | | | | | ASCT. Bridging therapy with gemcitabine/cisplatin |
| | | | | | | with no response. |
| 09 | 25 × 10⁶ | AITL | 61 | F | Low | 2 prior lines of treatment with ASCT: 1. |
| | | | | | | IEV/methotrexate achieving CR consolidated with |
| | | | | | | ASCT remained in remission for 24 months; On |
| | | | | | | relapse, received GDP without achieving response. |
| | | | | | | The patient did not receive bridging therapy. |
| 33 | 75 × 10⁶ | PTCL | 35 | F | Low | 1 prior line of treatment: 1. R-CHOP achieving CR |
| | | NOS | | | | for 26 months. The patient did not receive ASCT. |
| | | | | | | Bridging therapy with GDP with no response. |
| 25 | 75 × 10⁶ | PTCL | 53 | M | Low | 4 prior lines of treatment: 1. CHOP achieving SD, 2. |
| | | NOS | | | | ICE achieving PR for 3 months, 3. azacytidine |
| | | | | | | without response, 4. gemcitabine/oxaliplatin |
| | | | | | | achieving SD. No ASCT. |
| | | | | | | Bridging therapy with brentuximab with no |
| | | | | | | response. |
| 14 | 225 × 10⁶ | ALCL | 47 | M | Low | 3 prior lines of treatment with ASCT: 1. CHOP |
| | | | | | | achieving PR for 22 months, 2. BEAM/brentuximab |
| | | | | | | achieving CR for 4 months, 3. brentuximab |
| | | | | | | (unknown response). |
| | | | | | | Bridging therapy with brentuximab which resulted in |
| | | | | | | CMR. |
| 72 | 450 × 10⁶ | PTCL | 44 | M | Low | 2 prior lines of treatment with ASCT: 1. |
| | | NOS | | | | CHOEP/brentuximab achieving PR for 12 months, |
| | | | | | | 2. ICE without response. |
| | | | | | | Bridging therapy with |
| | | | | | | cisplatin/cytarabine/etoposide/methylprednisone |
| | | | | | | with no response. |
| 55 | 450 × 10⁶ | AITL | 63 | M | Low- | 3 prior lines of treatment: 1. CHOP achieving PR for |
| | | | | | Int | 1 month, 2. ICE without response, 3. duvelisib |
| | | | | | | achieving PR for 2 months. No ASCT. |
| | | | | | | Bridging therapy with vinblastine with response. |
| 59 | 450 × 10⁶ | PTCL | 58 | M | Low | 3 prior lines of treatment without ASCT: 1. CHOEP |
| | | NOS | | | | achieving PR for 4 months, 2. GDP achieving PR |
| | | | | | | for 2 months, 3. brentuximab achieving PR for 12 |
| | | | | | | month. No ASCT. |
| | | | | | | This patient did not receive bridging therapy. |
| 47 | 450 × 10⁶ | AITL | 61 | M | Low- | 2 prior lines of treatment: 1. CHOEP achieving CR |
| | | | | | Int | for 12 months, 2. DHAP achieving CR for 3 months. |
| | | | | | | No ASCT. |
| | | | | | | Bridging therapy with dexamethasone without |
| | | | | | | response. |

TABLE 13
CAR T cell product characteristics.

| | | Actual | TRBC1⁺ of | TRBC1⁺ | TRBC1⁺ | Manufacture | | | RQR8 |
|---|---|---|---|---|---|---|---|---|---|
| | | CAR T | Leukapheresis | post | RQR8⁺ | Process | | CAR T Cells | Transduction |
| Patient | | Cells | Starting | depletion | (CAR- | Duration | Viability | Total | Efficiency |
| ID | Dose | Infused | Material (%) | (%) | T %) | (Days) | (%) | manufactured | (%) |
| 1 | 25 × 10⁶ | 24 × 10⁶ | 24.9 | 0.3 | 0.2 | 9 | 96.8 | 460 × 10⁶ | 39.0 |
| 9 | 25 × 10⁶ | 23 × 10⁶ | 48.3 | 0.2 | 0.5 | 9 | 93.5 | 82 × 10⁶ | 27.2 |
| 22 | 25 × 10⁶ | 23 × 10⁶ | 41.7 | 0 | 0.1 | 8 | 98.0 | 153 × 10⁶ | 33.8 |
| 25 | 75 × 10⁶ | 58 × 10⁶ | 32.0 | 4.6 | 0.3 | 11 | 93.9 | 80 × 10⁶ | 7.0 |
| 33 | 75 × 10⁶ | 70 × 10⁶ | 42.7 | 1.1 | 0.2 | 11 | 97.0 | 354 × 10⁶ | 14.5 |
| 14 | 225 × 10⁶ | 190 × 10⁶ | 31.6 | 0.3 | 0.1 | 8 | 95.9 | 490 × 10⁶ | 44.8 |
| 47 | 450 × 10⁶ | 387 × 10⁶ | 27.9 | 5.6 | 0.1 | 10 | 96.5 | 709 × 10⁶ | 42.2 |
| 55 | 450 × 10⁶ | 403 × 10⁶ | 34.7 | 0.4 | 0.1 | 11 | 96.7 | 676 × 10⁶ | 47.7 |
| 59 | 450 × 10⁶ | 432 × 10⁶ | 48.1 | 0.6 | 0.0 | 11 | 98.0 | 1330 × 10⁶ | 34.1 |
| 72 | 450 × 10⁶ | 412 × 10⁶ | 29.9 | 0.5 | 0.0 | 11 | 97.2 | 654 × 10⁶ | 35.5 |

### Safety of AUTO4 Administration

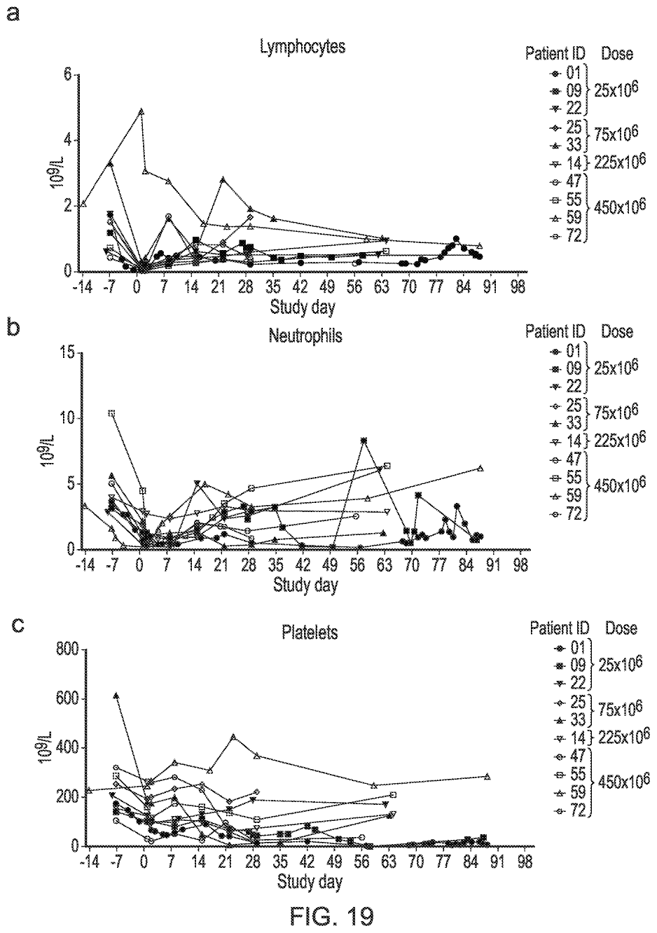

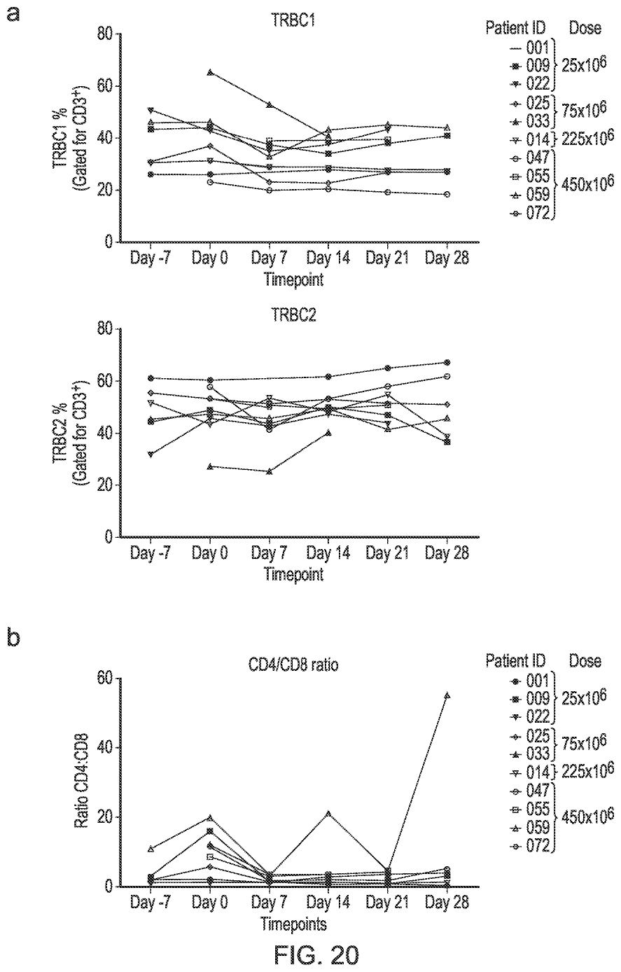

Treatment with Flu/Cy and AUTO4 was generally well tolerated (Table 14), Any grade cytokine release syndrome (CRS) was observed in 4/10 patients (all dosed at 450×10⁶) at a median onset of 1 day (range, 1-5), lasting a median duration of 2 days (range, 2-10). In 3/4 patients, CRS was Grade 1-2. One patient experienced grade 3 CRS on day 8 which resolved within 3 days. Tocilizumab was given to 2 patients; no steroids were administered in any patient to treat CRS. Importantly, no immune cell-associated neurotoxicity syndrome (ICANS) of any grade or dose limiting toxicity (DLT) were observed in any patient (Table 14). The most common additional treatment related adverse events irrespective of causality were transient neutropenia (grade 3 in 40%), thrombocytopenia (≥grade 3 in 20%), anaemia (≥grade 3 in 40%) and lymphopenia, consistent with effects expected from lymphodepletion chemotherapy. More pronounced, transient Grade 4 lymphopenia was observed after Flu/Cy lymphodepletion and AUTO4 infusion, but lymphocyte counts generally recovered to baseline within 3 weeks (FIG. 19). Unexpectedly, no alteration of peripheral blood (PB) TRBC1:TRBC2 ratio was seen, suggesting lack of CAR T mediated depletion of normal TRBC1+ T cells (FIG. 20).

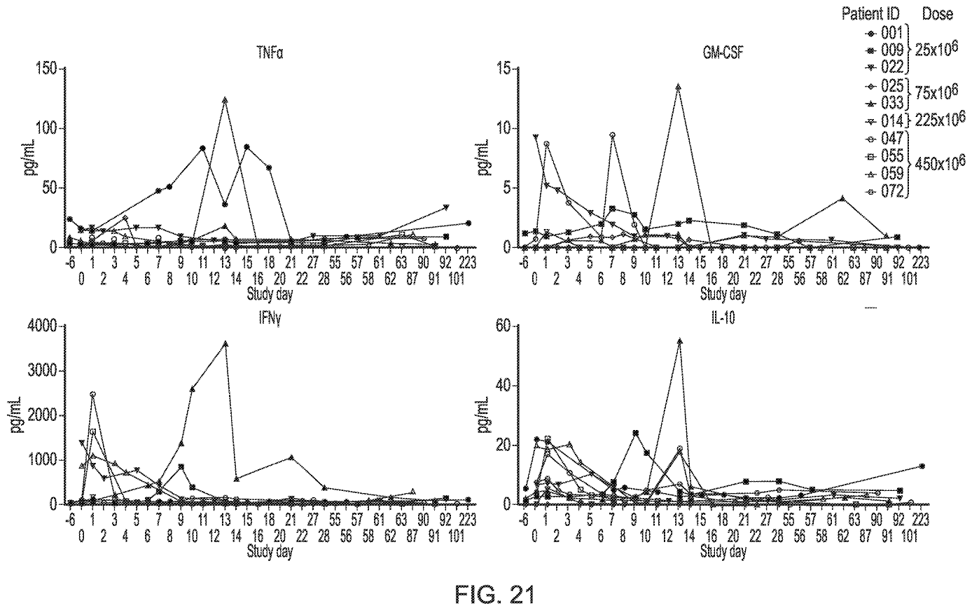

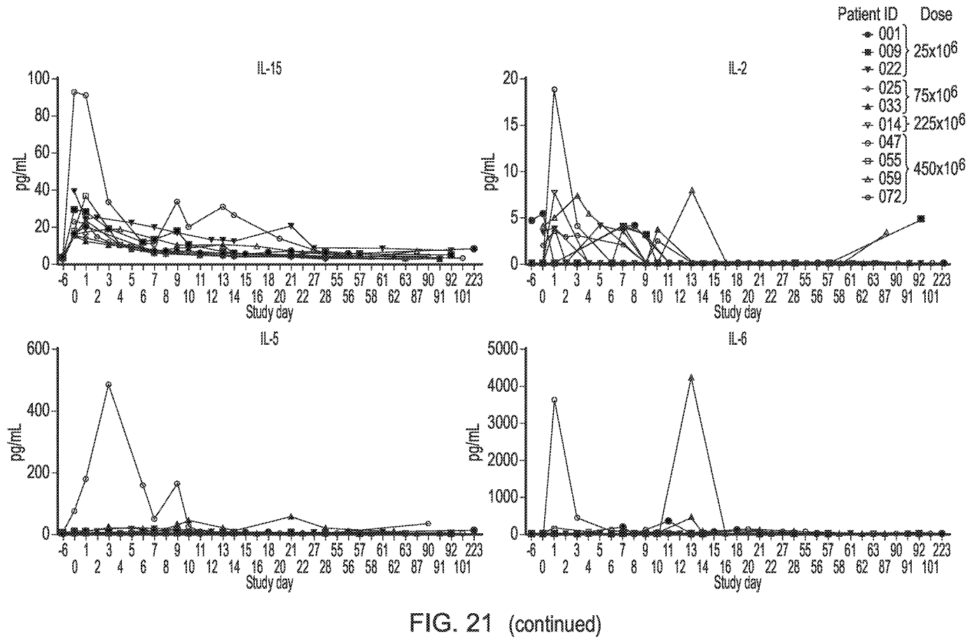

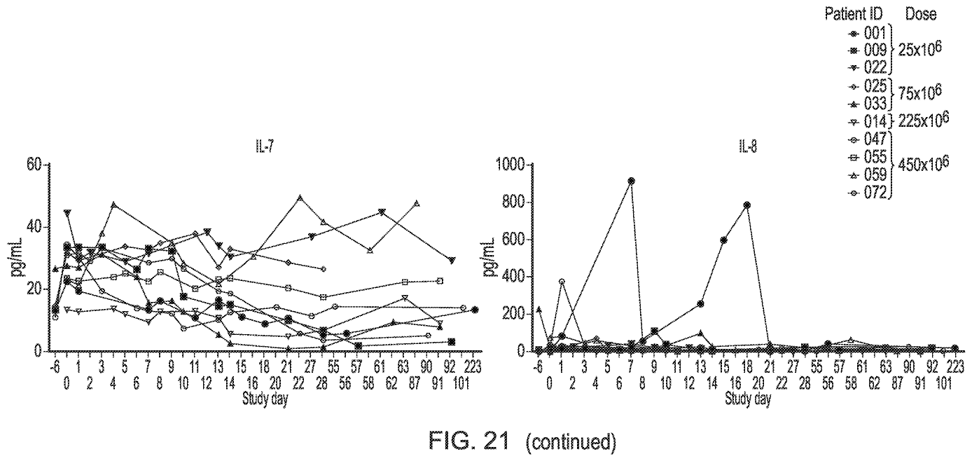

Serum cytokine levels were low across the study (FIG. 21). This is consistent with the low severity of CRS seen.

TABLE 14
Summary of adverse effects.

| | 25 × 10⁶ | 75 × 10⁶ | 225 × 10⁶ | 450 × 10⁶ | Total |
|---|---|---|---|---|---|
| | (n = 3) | (n = 2) | (n = 1) | (n = 4) | (n = 10) |
| Dose Limiting Toxicity | 0 | 0 | 0 | 0 | 0 |
| (DLT) | | | | | |
| Any Grade Neutropenia | 3 (100%) | 2 (100%) | 0 | 3 (75%) | 8 (80%) |
| Any Grade Infections | 3 (100%) | 1 (50%) | 1 (50%) | 1 (25%) | 6 (60%) |
| Any Grade CRS | 0 | 0 | 0 | 4 (100%) | 4 (40%) |
| Grade 3 CRS | 0 | 0 | 0 | 1 (25%) | 1 (10%) |
| Any grade ICANS | 0 | 0 | 0 | 0 | 0 |

Two patients experienced rise in Epstein Barr virus (EBV) genomic DNA copy number. One patient (dosed at 25×10⁶) developed grade 2 EBV reactivation on study day 27 in the context of immune mediated thrombocytopenia. Rituximab was administered (once per week) on day 78 and the event resolved on day 93. Another patient (dosed at 75×10⁶) developed grade 1 EBV infection on day 29 post AUTO4 infusion which was still DNA positive at last follow up. No treatment was administered to this patient.

### Disease Response Following AUTO4 Infusion

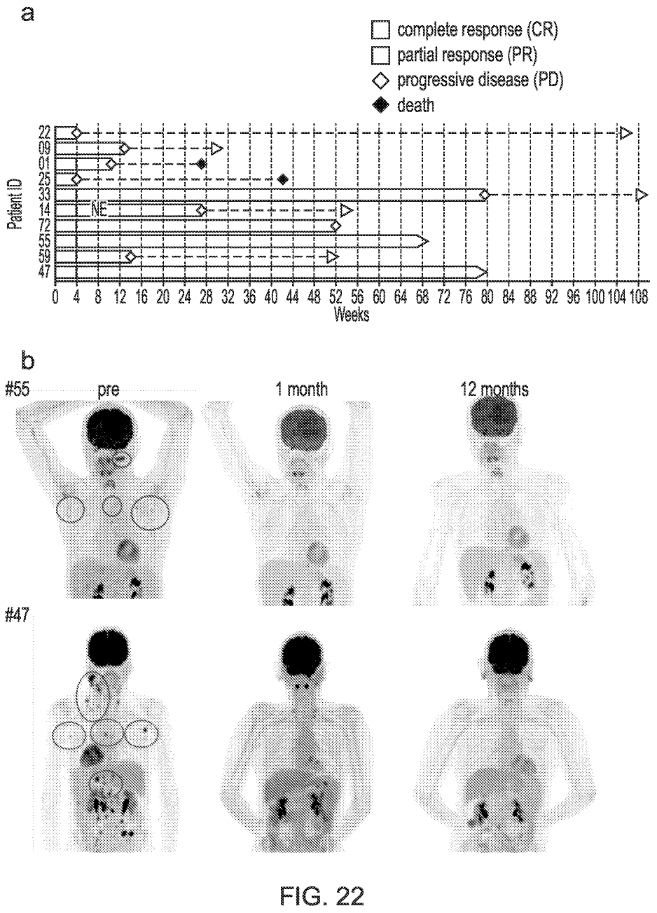

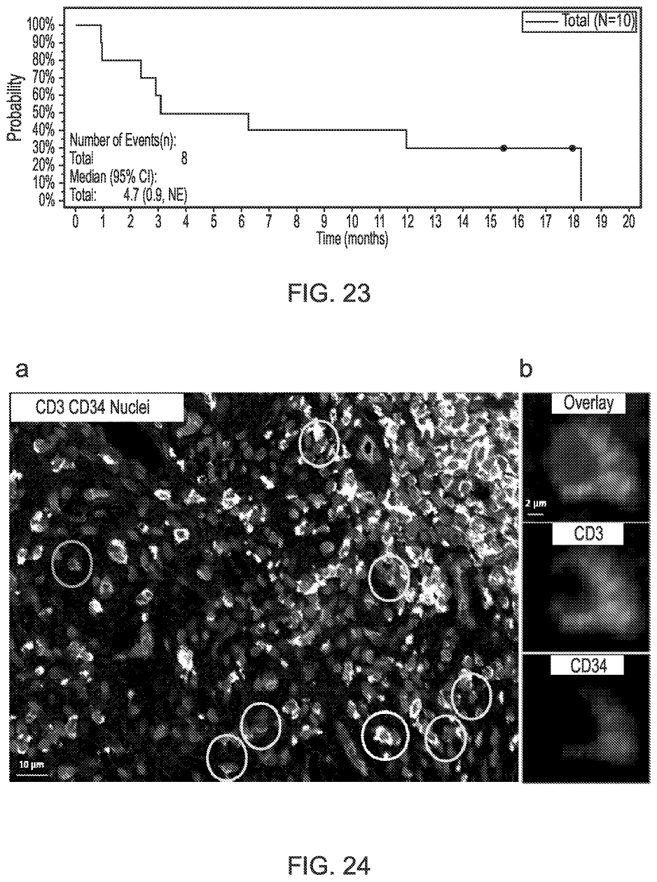

Median follow-up was 13.8 months at the data cut-off date (28 Apr. 2023). One patient who received 225×10⁶ CAR T cells achieved complete metabolic response (CMR) by PET-CT after bridging therapy, so response at month 1 was evaluable in only 9/10 patients. Individual patient outcomes are illustrated in FIG. 22a. The best overall response rate (ORR=CR+PR) at any time post AUTO4 infusion by PET-CT among all response evaluable patients was 66.6% (6/9 patients). CMR was observed in 4 of 6 responding patients and 2 patients achieved PR. At the highest dose level (450×10⁶ CAR T cells), all 4 patients achieved an objective response with 3 patients achieving CMR and 1 PR by PET-CT. Among patients in CMR at month 1, one patient was dosed at 25×10⁶ dose level (relapsed at month 2 and did not receive further treatments until death due to underlying disease at study day 190), and 3 patients were dosed at the highest dose level tested (450×10⁶). Two of these 3 patients are in ongoing remission at 15 and 18 months respectively, having received no further anti-lymphoma therapy (FIG. 22b). Interestingly, both patients had AITL. Of note, one of these two patients had disease which was refractory to all 3 prior lines of therapy (Extended Data Table 4). The third patient at the highest dose level tested relapsed at month 3 after sustaining a CMR at day 28. Two infused patients (#72 and #33) who had achieved a PR did not require any further therapy with follow-up ≥12 months, but then showed disease progression at 12 and 18 months, respectively. Three patients had no response. The median duration of remission (DOR) in all responding patients across all cohorts was 5.3 months (95% CI 1.4, NE). Among all infused patients, median progression free survival (PFS) was 4.7 months (95% CI 0.9, NE) and median overall survival (OS) was not reached, with 90% (95% CI 47.3, 98.5) and 78.8% (95% CI 38.1, 94.3) projected to at month 9 and 18 respectively. Among all infused patients, 2 patients died due to the underlying disease at study day 190 and 294 and 8/10 are alive at the last follow up (FIG. 23).

### AUTO4 Expansion and Persistence

AUTO4 CAR-T expansion and persistence were assessed in all infused patients in peripheral blood (PB) by flow cytometry and digital droplet polymerase chain reaction (ddPCR), and in lymph nodes by ddPCR and immunofluorescence (IF). At the 25, 75 and 225×10⁶ dose levels, no expansion was detected in PB. At the highest dose level of 450×10⁶, one patient had detectable transgene in peripheral blood with 1476 vector copy number (VCN)/ug DNA in PB 10 minutes post-infusion. The value dropped to 156 VCN/ug DNA by day 7 and 76 VCN/ug DNA by day 13. Day 123 was the latest time point in which CAR-T cells were detectable by ddPCR (70 VCN/ug DNA; limit of quantification 20 copies/ug DNA). No CAR-T cells were detected by flow cytometry.

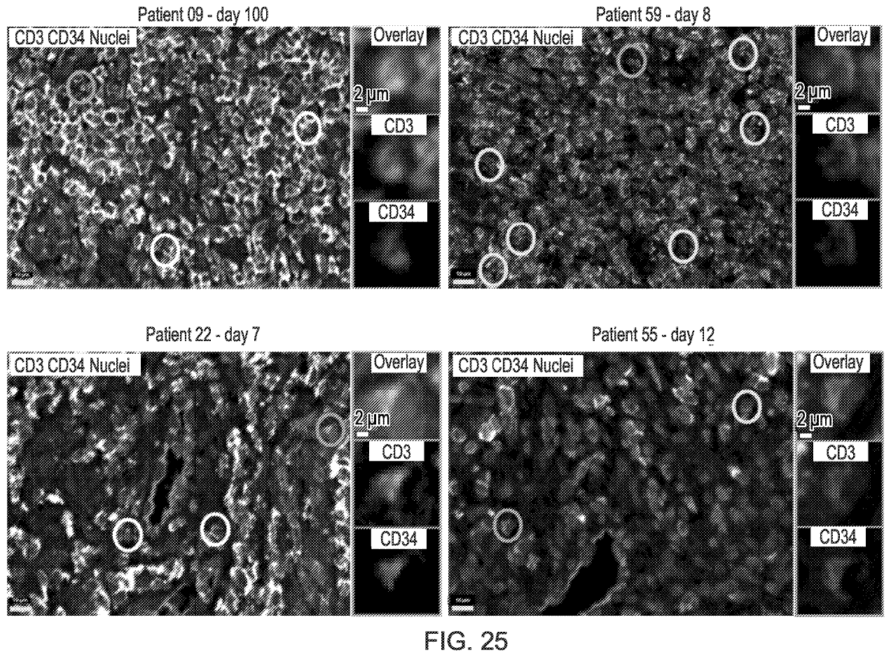

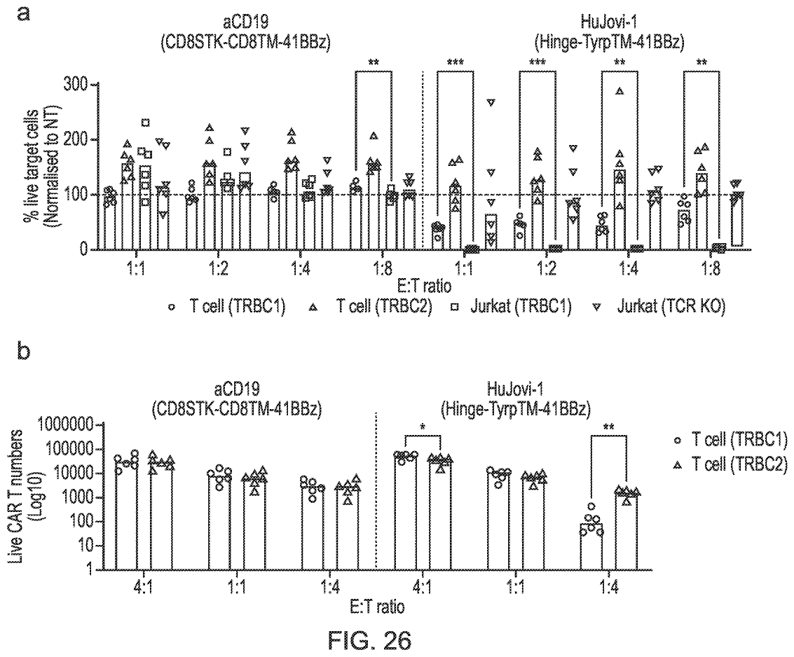

The clinical study included on-treatment biopsies of sites of disease after AUTO4 administration when feasible. In contrast to findings in PB, in all 5 patients (5/5) with post-treatment lymph nodes accessible to biopsy and suitable for testing (median 11 days post CAR-T, range 7-279 days), both IF and ddPCR confirmed infiltration of AUTO4 CAR-T cells at these tumor sites). Lymph node ddPCR AUTO4 copy number ranged from 111 to 171,700 VCN/ug DNA. IF biopsy imaging is shown in FIG. 24 and FIG. 25. PCR quantification of integrant copy number and IF counting is shown in Table 15.

TABLE 15
CAR T detection in lymph nodes by
PCR and by immunofluorescence.

| | | Timepoint | | % CAR per |
|---|---|---|---|---|
| Infusion | | (post- | | total CD3⁺ |
| dosage | Patient ID | infusion) | VCN/μg DNA | cells (IF) |
| 25 × 10⁶ | 09 | Day 100 | 520.15 | 2.71 |
| | 22 | Day 7 | 4141 | 1.20 |
| | 01 | Day 19 | 19,695 | 4.41 |
| 75 × 10⁶ | 33 | Biopsy not taken | | |
| | 25 | Day 28 | Tissue exhausted | |
| 225 × 10⁶ | 14 | Biopsy not taken | | |
| 450 × 10⁶ | 47 | Biopsy not taken | | |
| | 59 | Day 8 | 2,525 | 4.06 |
| | 55 | Day 12 | 10,302 | 1.39 |

| | | |
|---|---|---|
| 72 | 62 | Low quality sample |

### Summary of Examples 19 and 20

AUTO4 was safe with minimal toxicity observed. Any grade CRS was observed in 4/10 patients (all at 450×10⁵). One patient (450×10⁶ cohort) developed grade 3 CRS which resolved within 3 days. Importantly, no ICANS of any grade or dose limiting toxicity (DLT) were seen. This lack of immunotoxicity correlated with low levels of serum cytokines. Two cases of EBV reactivation were observed. However, giving the late timing of these reactivations and lack of CAR T persistence and lymphopenia at the time, these are unlikely to be caused by AUTO4 CAR T cells.

The preliminary efficacy and the ongoing responses reported in this phase 1 study are encouraging, with most responses observed at the highest AUTO4 dose: Three of 4 patients who received the 450×10⁶ CAR T cells (highest dose) achieved CMR at month 1 and two of them remain in CMR beyond 18 months, suggesting a potential for AUTO4 CAR-T to induce long-lasting remissions in a proportion of patients. Two patients with partial response are alive beyond 12 months without receiving any new anti-cancer medication. Furthermore, a possible impact on survival was seen, with 8/10 patients alive at last follow up and median duration of overall survival of 13.8 months, compared to a historical average of <6 months in this patient cohort.

1. A method for treating a TRBC1-positive malignancy in a patient comprising administering to the patient autologous anti-TRBC1 CAR T-cells.

2. The method of claim 1 wherein the age of the patient is eighteen years or older.

3. The method of claim 1 or 2 wherein the TRBC1-positive malignancy is: peripheral T-cell lymphoma not otherwise specified (PTCL-NOS), angio-immunoblastic T-cell lymphoma (AITL), or anaplastic large cell lymphoma (ALCL).

4. The method of claim 1 or 2 wherein the TRBC1-positive malignancy is enteropathy-associated T-cell lymphoma (EATL), hepatosplenic T-cell lymphoma (HSTL), extranodal NK/T-cell lymphoma nasal type, cutaneous T-cell lymphoma, primary cutaneous ALCL, T cell prolymphocytic leukaemia and T-cell acute lymphoblastic leukaemia.

5. The method of any preceding claim wherein the patient has:
a) relapsed or refractory T-cell malignancy following at least one line of therapy, and
b) confirmed TRBC1-positive tumour.

6. The method of any preceding claim wherein the patient is administered a single dose of about 25×10⁶, 75×10⁶, 225×10⁶, 450×10⁶, or 900×10⁶ anti-TRBC1 CAR T cells.

7. The method of claim 6 wherein the patient is administered a single dose of 450×10⁶ anti-TRBC1 CAR T cells.

8. The method of any preceding claim wherein the administration is an intravenous injection through a Hickman line or peripherally inserted central catheter.

9. The method of any preceding claim wherein the patient receives a pre-conditioning chemotherapy.

10. The method of claim 1 wherein the anti-TRBC1 CAR T-cells express a CAR comprising a TRBC1-binding domain which comprises
a) a heavy chain variable region (VH) having
CDRs with the following sequences:
VH CDR1:
(SEQ ID NO: 1)
GYTFTGY,
VH CDR2:
(SEQ ID NO: 2)
NPYNDD
and
VH CDR3:
(SEQ ID NO: 3)
GAGYNFDGAYRFFDF;
and
b) a light chain variable region (VL) having
CDRs with the following sequences:
VL CDR1:
(SEQ ID NO: 4)
RSSQRLVHSNGNTYLH,
VL CDR2:
(SEQ ID NO: 5)
RVSNRFP
and
VL CDR3:
(SEQ ID NO: 6)
SQSTHVPYT.

11. The method of claim 10, wherein the anti-TRBC1 binding domain comprises a VH domain having the sequence shown as SEQ ID NO: 9, or a variant thereof with at least 95% sequence identity.

12. The method of claim 10, wherein the anti-TRBC1 binding domain comprises a VL domain having the sequence shown as SEQ ID NO: 19, or a variant thereof with at least 95% sequence identity.

13. The method of claim 10, wherein the anti-TRBC1 binding domain comprises a VH domain having the sequence shown as SEQ ID NO: 9 and a VL domain having the sequence shown as SEQ ID NO: 19, or a variant thereof with at least 95% sequence identity.

14. The method of claim 10, wherein the anti-TRBC1 domain comprises the six CDRs grafted on to a human antibody framework.

15. The method of any preceding claim, wherein the CAR comprises the anti-TRBC1 binding domain and a transmembrane domain connected by a spacer.

16. The method of claim 15, wherein the spacer comprises a human IgG1 hinge.

17. The method of any preceding claim wherein the CAR comprises an intracellular T cell signaling domain comprising a 41BB endodomain and a CD3-Zeta endodomain.

18. The method of claim 10 wherein the CAR comprises an anti-TRBC1 binding domain comprises a VH domain having the sequence shown as SEQ ID NO: 9 and a VL domain having the sequence shown as SEQ ID NO: 19, a human IgG1 hinge, and an intracellular T cell signaling domain comprising a 41 BB endodomain and a CD3-Zeta endodomain.

19. The method of claim 10 wherein the CAR comprises the sequence shown in SEQ ID NO: 35.
